(19) United States
(12) Patent Application Publication
(10) Pub. No.: US 2025/0361263 A1
(43) Pub. Date: Nov. 27, 2025

(54) C17 POLAR-SUBSTITUTED HETEROAROMATIC SYNTHETIC TRITERPENOIDS AND METHODS OF USE THEREOF

(71) Applicant: REATA PHARMACEUTICALS, INC., Irving, TX (US)

(72) Inventors: Xin JIANG, Coppell, TX (US); Christopher F. BENDER, Garland, TX (US); Ha DO, Irving, TX (US); Haizhou SUN, Irving, TX (US); Melean VISNICK, Irving, TX (US)

(73) Assignee: REATA PHARMACEUTICALS, INC., Irving, TX (US)

(21) Appl. No.: 19/231,055

(22) Filed: Jun. 6, 2025

Related U.S. Application Data
(63) Continuation of application No. 16/933,635, filed on Jul. 20, 2020, now Pat. No. 12,344,631.
(60) Provisional application No. 62/876,467, filed on Jul. 19, 2019.
(60) Provisional application No. 62/952,048, filed on Dec. 20, 2019.

(51) Int. Cl. C07J 53/00; A61K 9/00

(52) U.S. Cl. CPC C07J 53/002; A61K 9/0014; A61K 9/0019; A61K 9/0053

(57) ABSTRACT

Disclosed herein are compounds of the formula:

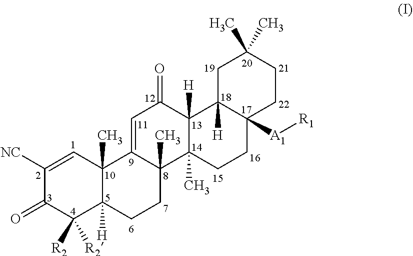

wherein the variables are defined herein. Also provided are pharmaceutical compositions thereof. In some aspects, the compounds and compositions provided herein may be used as antioxidant inflammation modulators. In some aspects, the present disclosure provides methods wherein the compounds and composition described herein are used for the treatment of diseases and disorders, including those associated with inflammation and cancer.

This application is a continuation of U.S. application Ser. No. 16/933,635, filed Jul. 20, 2020, which claims the benefit of priority to U.S. Provisional Application No. 62/876,467, filed on Jul. 19, 2019, and 62/952,048, filed on Dec. 20, 2019, the entire contents of each of which are hereby incorporated by reference.

### BACKGROUND OF THE INVENTION

### I. Field of the Invention

The present invention relates generally to the fields of biology, chemistry, and medicine. More particularly, it concerns compounds, compositions and methods for the treatment and prevention of diseases and disorders such as those associated with oxidative stress and inflammation.

### II. Description of Related Art

The anti-inflammatory and anti-proliferative activity of the naturally occurring triterpenoid, oleanolic acid, has been improved by chemical modifications. For example, 2-cyano-3,12-diooxooleana-1,9(11)-dien-28-oic acid (CDDO) and related compounds have been developed (Honda et al., 1997; Honda et al., 1998; Honda et al., 1999; Honda et al., 2000a; Honda et al., 2000b; Honda, et al., 2002; Suh et al. 1998; Suh et al., 1999; Place et al., 2003; Liby et al., 2005; and U.S. Pat. Nos. 6,326,507, 6,974,801, 7,435,755, 7,795,305, 7,863,327, 7,915,402, 7,943,778, 8,034,955, 8,071,632, 8,124,656, 8,124,799, 8,129,429, 8,338,618, 8,394,967, 8,440,820, 8,440,854, 8,455,544, 8,586,775, 8,993,640, 9,090,574, 9,102,681, 9,249,089, 9,278,912, 9,278,913, 9,290,536, 9,593,074, 9,701,709, 9,512,094, 9,556,222, 9,670,147, 9,757,359, 9,856,286, 9,889,143, 10,093,614, 10,105,372, 10,398,711, 10,501,489, or 10,556,858). Bardoxolone methyl (CDDO-Me; RTA 402) and omaveloxolone (RTA 408), have been evaluated clinically, including, for example, for the treatment of cancer, chronic kidney disease, pulmonary arterial hypertension, and Friedreich's Ataxia (Pergola et al., 2011; Hong et al., 2012; U.S. Pat. No. 8,993,640).

Synthetic triterpenoid analogs of oleanolic acid (OA) have also been shown to be inhibitors of cellular inflammatory processes, such as the induction by IFN-γ of inducible nitric oxide synthase (iNOS) and of COX-2 in mouse macrophages. See Honda et al. (2000a); Honda et al. (2000b), and Honda et al. (2002). Synthetic derivatives of another triterpenoid, betulinic acid, have also been shown to inhibit cellular inflammatory processes, although these compounds have been less extensively characterized (Honda et al., 2006). The pharmacology of these synthetic triterpenoid molecules is complex. Compounds derived from oleanolic acid have been shown to affect the function of multiple protein targets and thereby modulate the activity of several important cellular signaling pathways related to oxidative stress, cell cycle control, and inflammation (e.g., Dinkova-Kostova et al., 2005; Ahmad et al., 2006; Ahmad et al., 2008; Liby et al., 2007a). Derivatives of betulinic acid, though they have shown comparable anti-inflammatory properties, also appear to have significant differences in their pharmacology compared to OA-derived compounds (Liby et al., 2007b). Given that the biological activity profiles of known triterpenoid derivatives vary, and in view of the wide variety of diseases that may be treated or prevented with compounds having potent antioxidant and anti-inflammatory effects, and the high degree of unmet medical need represented within this variety of diseases, it is desirable to synthesize new compounds with diverse structures that may have improved biological activity profiles for the treatment of one or more indications.

### SUMMARY OF THE INVENTION

The present disclosure provides novel synthetic triterpenoid derivatives with anti-inflammatory and/or antioxidant properties, pharmaceutical compositions, and methods for their manufacture, and methods for their use.

In one aspect, there are provided compounds of the formula:

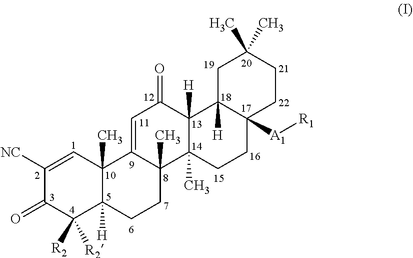

wherein:
A₁ is -heteroarenediyl_{(C≤3)}-;
R₁ is a polar-substituted alkyl_{(C≤3)}; and
R₂ and R₂′ are each independently hydrogen or methyl;
or a pharmaceutically acceptable salt thereof.

In some embodiments, the compounds are further defined:

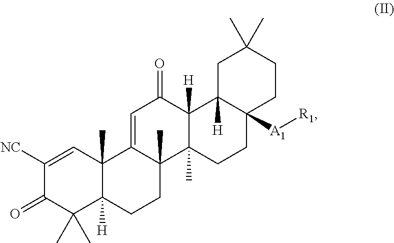

wherein:
A₁ is -heteroarenediyl_{(C≤3)}-; and
R₁ is a polar-substituted alkyl_{(C≤3)};
   or a pharmaceutically acceptable salt thereof.

In some embodiments, the compounds are further defined:

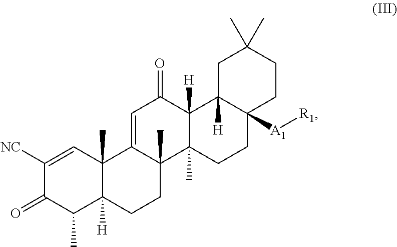

wherein:
A₁ is -heteroarenediyl_{(C≤3)}-;
R₁ is a polar-substituted alkyl_{(C≤3)}; and
   or a pharmaceutically acceptable salt thereof.

In some embodiments, -A₁-R₁ is:

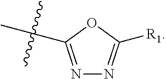

In other embodiments, -A₁-R₁ is:

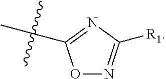

In still other embodiments, -A₁-R₁ is:

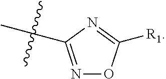

In yet other embodiments, -A₁-R₁ is:

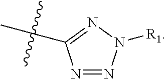

In some embodiments, R₁ is polar-substituted ethyl. In other embodiments, R₁ is polar-substituted methyl. In some embodiments, R₁ is monopolar-substituted alkyl_{(C≤3)}. In further embodiments, R₁ is monopolar-substituted ethyl. In other embodiments, R₁ is monopolar-substituted methyl. In some embodiments, R₁ is monoaminoalkyl_{(C≤3)}, monofluoroalkyl_{(C≤3)}, or monohydroxyalkyl_{(C≤3)}. In some embodiments, R₁ is monoaminoalkyl_{(C≤3)}, such as 2-aminoethyl, or aminomethyl. In other embodiments, R₁ is monofluoroalkyl_{(C≤3)}, such as 2-fluoroethyl or fluoromethyl. In still other embodiments, R₁ is monohydroxyalkyl_{(C≤3)}, such as 2-hydroxyethyl or hydroxymethyl. In yet other embodiments, R₁ is —CH₂CH₂NHC(O)OCH₃, —CH₂CH₂NHC(O)NHCH₂CH₃, or —CH₂CH₂NHC(O)CH₃.

In some embodiments, -A₁-R₁ is

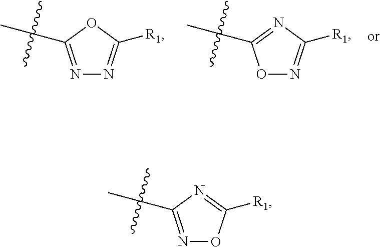

R₁ is aminomethyl, fluoromethyl, or hydroxymethyl, R₂ is hydrogen or methyl, and R₂′ is methyl. In some of these embodiments, -A₁-R₁ is

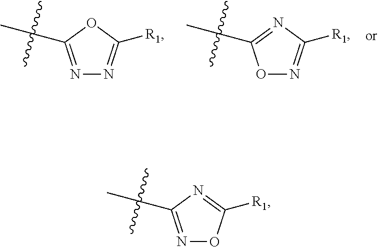

R₁ is fluoromethyl, R₂ is hydrogen or methyl, and R₂′ is methyl.

It is to be understood that the present invention specifically relates to each and every combination of features and embodiments described herein, including any combination of general and/or specific features/embodiments. In particular, the invention specifically relates to each combination of meanings (including general and/or specific meanings) for the various groups and variables comprised in formula (I).

In some embodiments, the compound is further defined as:

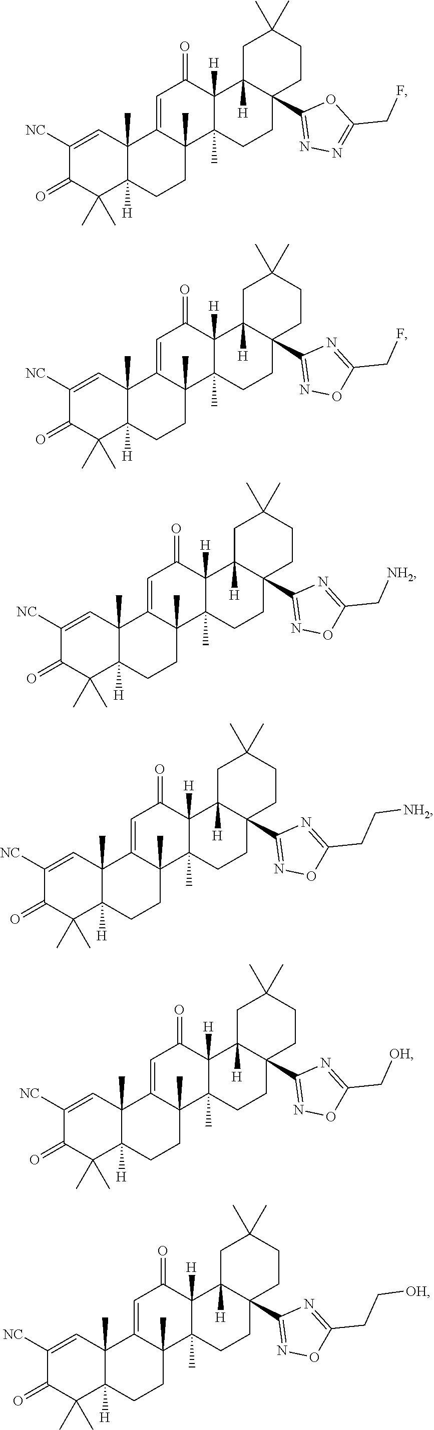

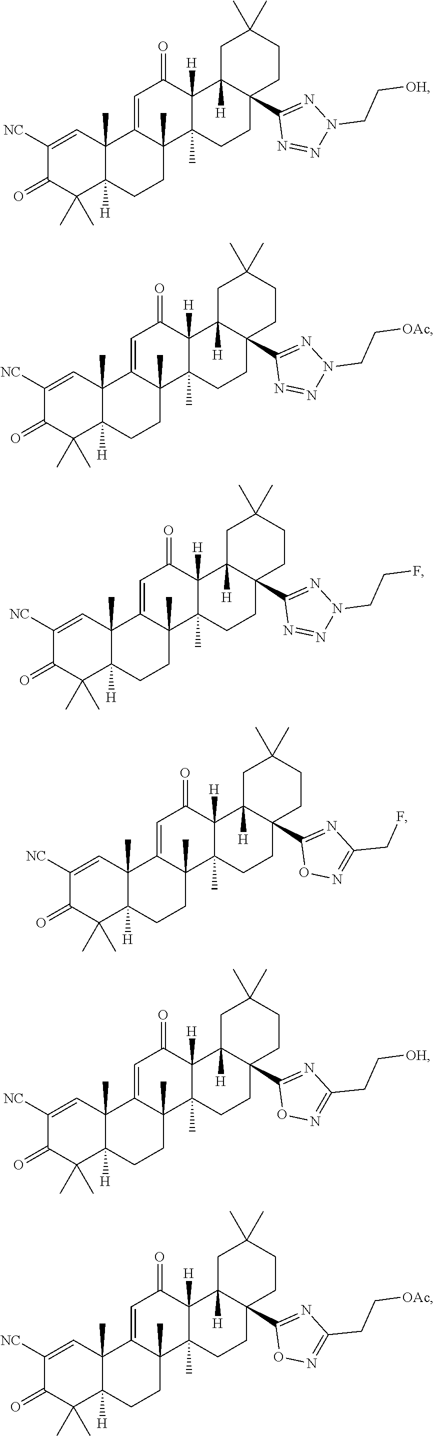

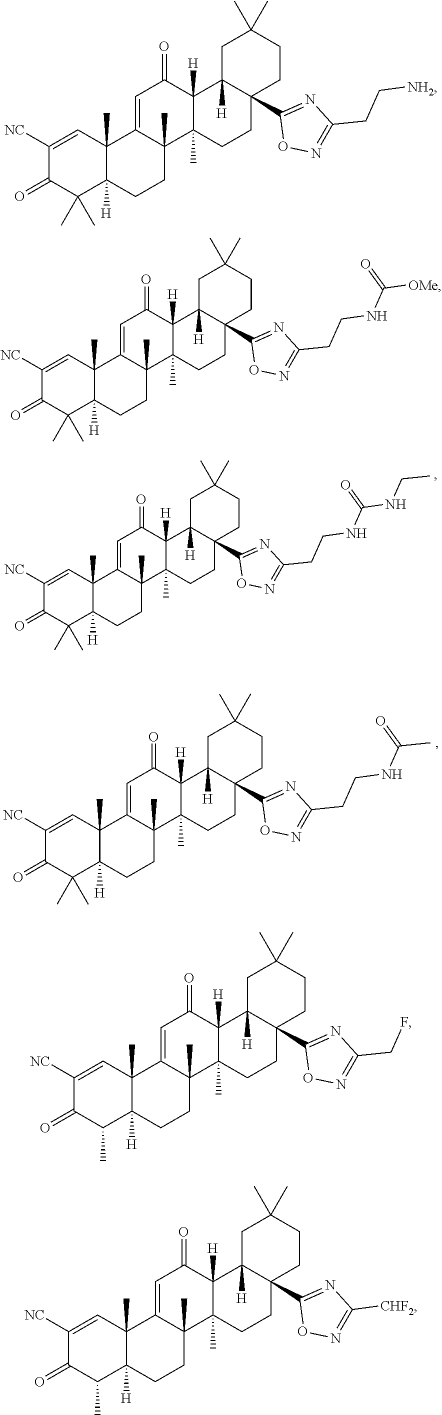

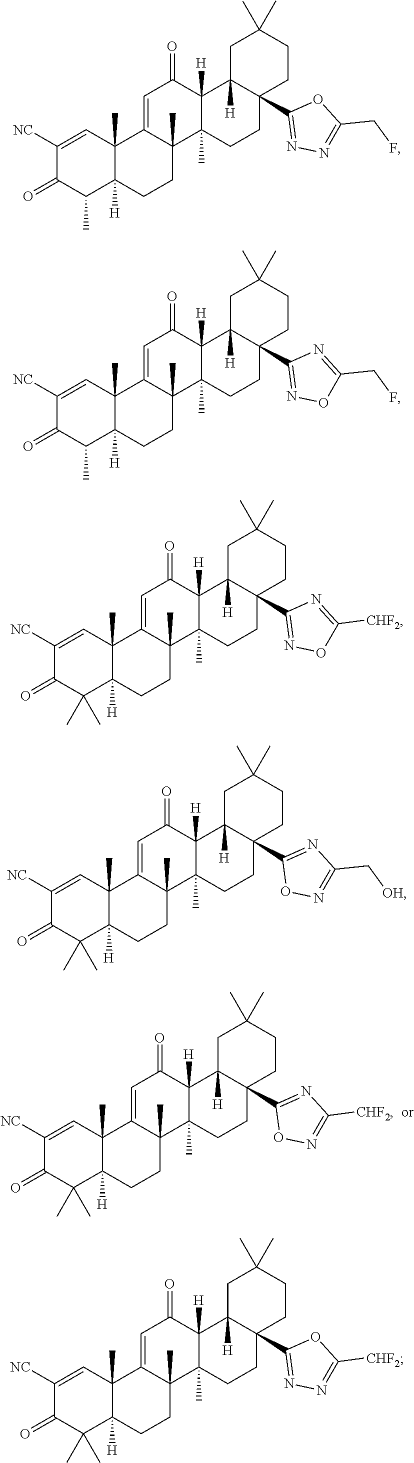

or a pharmaceutically acceptable salt of any of these formulas.

In further embodiments, the compound is further defined as:

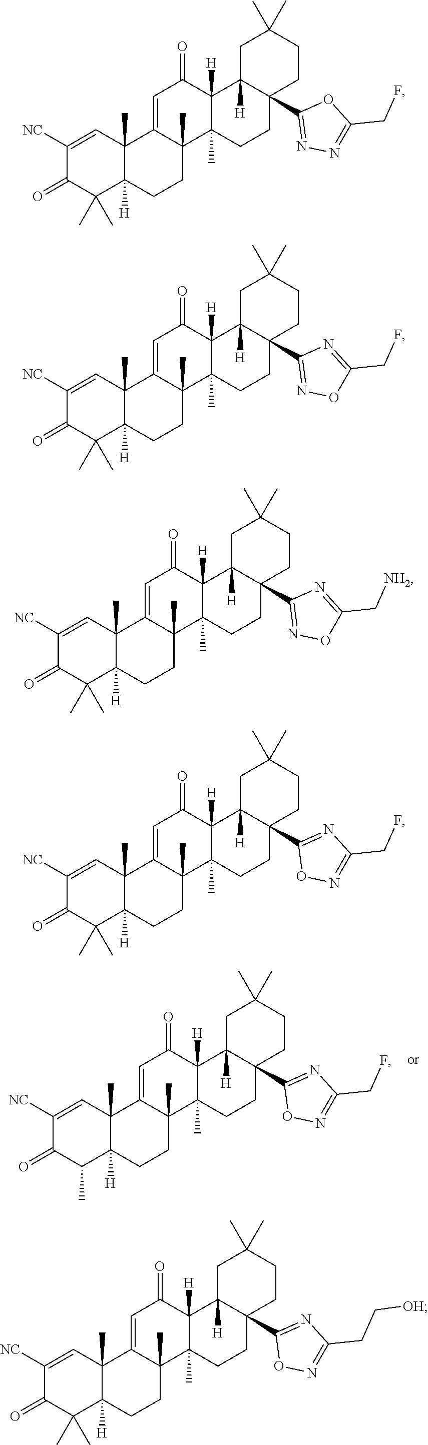

or a pharmaceutically acceptable salt of any of these formulas.

In further embodiments, the compound is further defined as:

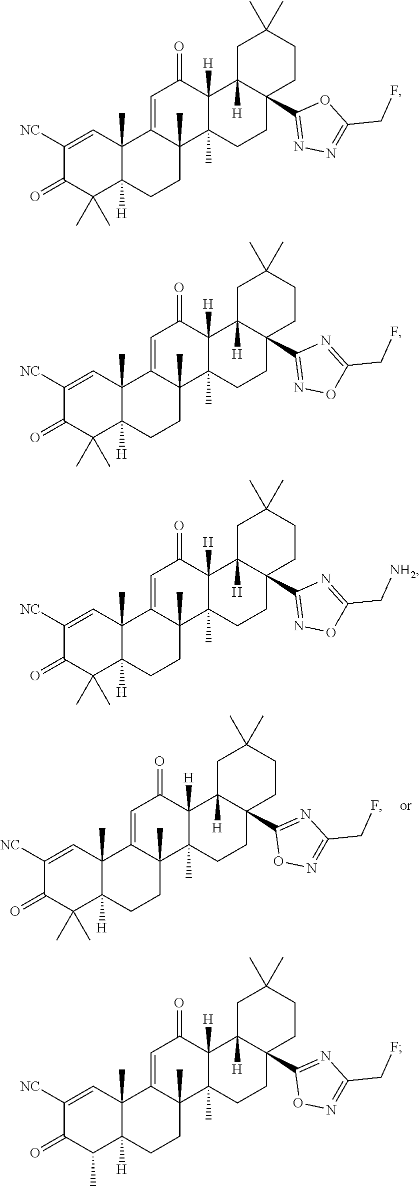

or a pharmaceutically acceptable salt of any of these formulas.

In yet further embodiments, the compound is further defined as:

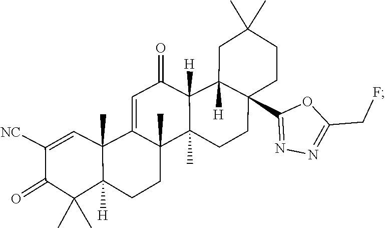

or a pharmaceutically acceptable salt thereof.

In other embodiments, the compound is further defined as:

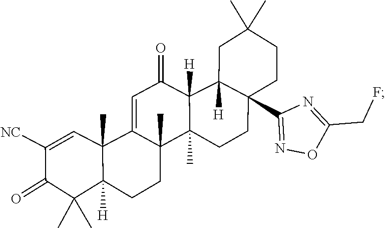

or a pharmaceutically acceptable salt thereof.

In still other embodiments, the compound is further defined as:

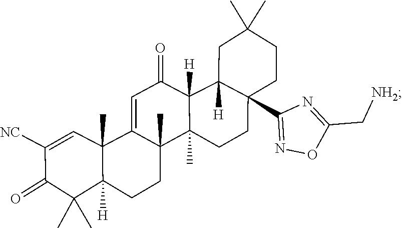

or a pharmaceutically acceptable salt thereof.

In yet other embodiments, the compound is further defined as:

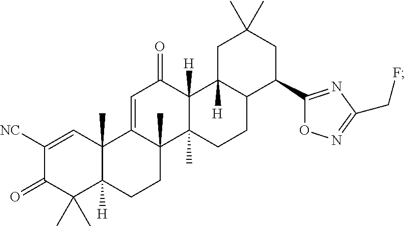

or a pharmaceutically acceptable salt thereof.

In other embodiments, the compound is further defined as:

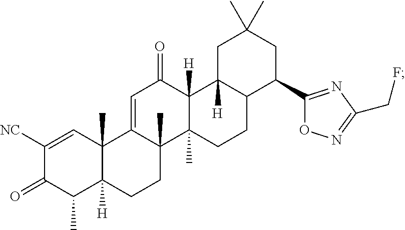

or a pharmaceutically acceptable salt thereof.

In still other embodiments, the compound is further defined as:

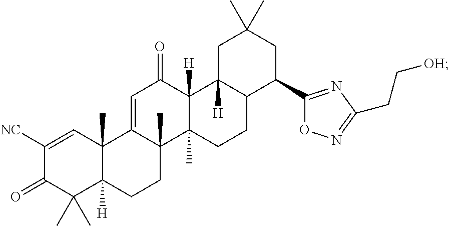

or a pharmaceutically acceptable salt thereof.

In some embodiments, the compound is further defined as:
(4aR,6aS,6bR,8aS,12aS,12bR,14bS)-8a-(5-(fluoromethyl)-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(fluoromethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(aminomethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(2-aminoethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(hydroxymethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(2-hydroxyethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(2-(2-hydroxyethyl)-2H-tetrazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
2-(5-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-2H-tetrazol-2-yl)ethyl acetate;
(4aR,6aS,6bR,8aS,12aS,12bR,14bS)-8a-(2-(2-fluoroethyl)-2H-tetrazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(fluoromethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(2-hydroxyethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
2-(5-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1,2,4-oxadiazol-3-yl)ethyl acetate;
(4aR,6aS,6bR,8aS,12aS,12bR,14bS)-8a-(3-(2-aminoethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
methyl (2-(5-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1,2,4-oxadiazol-3-yl)ethyl)carbamate;
1-(2-(5-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1,2,4-oxadiazol-3-yl)ethyl)-3-ethylurea;
N-(2-(5-((4aS,6aR,6bS,8aR,12aS,14aR,14bS)-11-cyano-2,2,6a,6b,9,9,12a-heptamethyl-10,14-dioxo-1,3,4,5,6,6a,6b,7,8,8a,9,10,12a,14,14a,14b-hexadecahydropicen-4a(2H)-yl)-1,2,4-oxadiazol-3-yl)ethyl)acetamide;
(4S,4aS,6aS,6bR,8aS,12aS,12bR,14bR)-8a-(3-(fluoromethyl)-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-8a-(3-(difluoromethyl)-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-8a-(5-(fluoromethyl)-1,3,4-oxadiazol-2-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-8a-(5-(fluoromethyl)-1,2,4-oxadiazol-3-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(difluoromethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(hydroxymethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(difluoromethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile; or
(4aR,6aS,6bR,8aS,12aS,12bR,14bS)-8a-(5-(difluoromethyl)-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In further embodiments, the compound is further defined as:
(4aR,6aS,6bR,8aS,12aS,12bR,14bS)-8a-(5-(fluoromethyl)-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(fluoromethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(aminomethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(fluoromethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-8a-(3-(fluoromethyl)-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile; or
(4aR,6aS,6bR,8aS,12aS,12bR,14bS)-8a-(3-(2-hydroxyethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In still further embodiments, the compound is further defined as:
(4aR,6aS,6bR,8aS,12aS,12bR,14bS)-8a-(5-(fluoromethyl)-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(fluoromethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-(aminomethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(fluoromethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile; or
(4S,4aS,6aS,6bR,8aS,12aS,12bR,14bR)-8a-(3-(fluoromethyl)-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile.

In some embodiments, the compound is further defined as:
(4aR,6aS,6bR,8aS,12aS,12bR,14bS)-8a-(5-(fluoromethyl)-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In other embodiments, the compound is further defined as:
(4aR,6aS,6bR,8aS,12aS,12bR,14bS)-8a-(5-(fluoromethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In still other embodiments, the compound is further defined as:
(4aR,6aS,6bR,8aS,12aS,12bR,14bS)-8a-(5-(aminomethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In yet other embodiments, the compound is further defined as:
(4aR,6aS,6bR,8aS,12aS,12bR,14bS)-8a-(3-(fluoromethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In other embodiments, the compound is further defined as:
(4S,4aS,6aS,6bR,8aS,12aS,12bR,14bR)-8a-(3-(fluoromethyl)-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile.

In other embodiments, the compound is further defined as:
(4aR,6aS,6bR,8aS,12aS,12bR,14bS)-8a-(3-(2-hydroxyethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In another aspect, the present disclosure provides compounds of the formula:

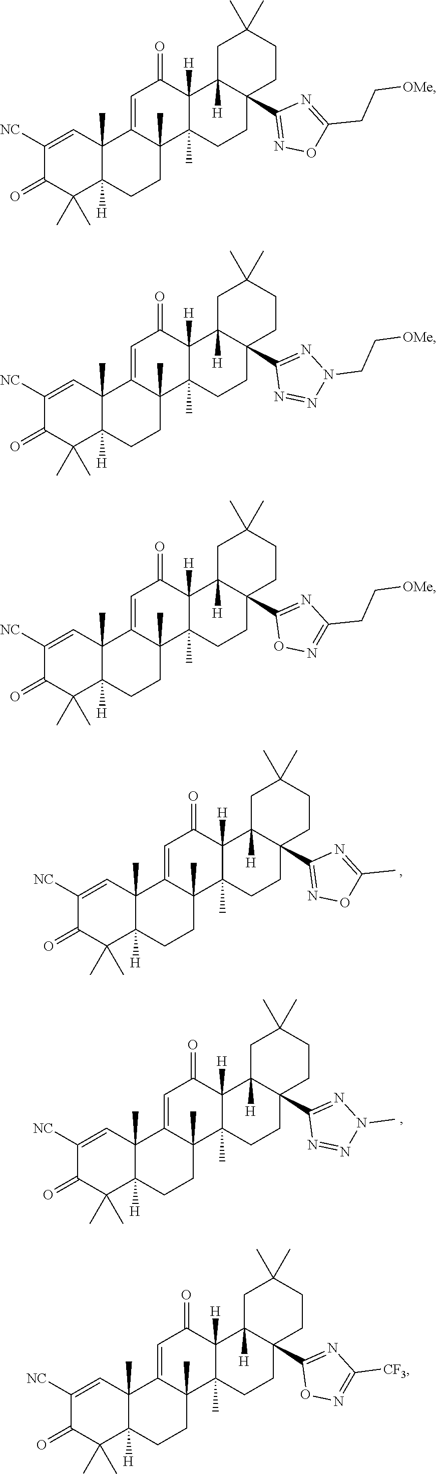

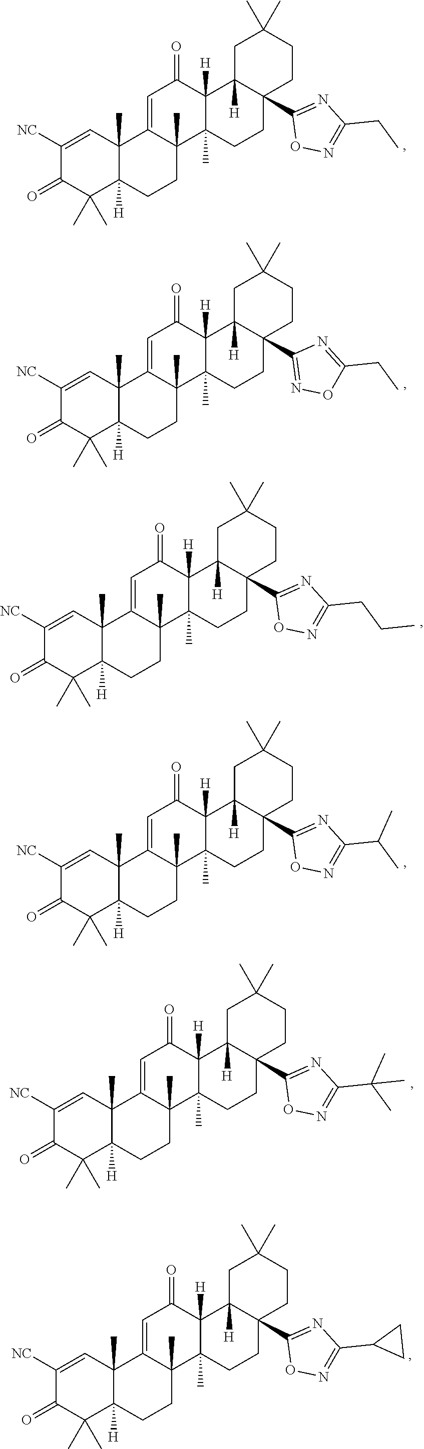

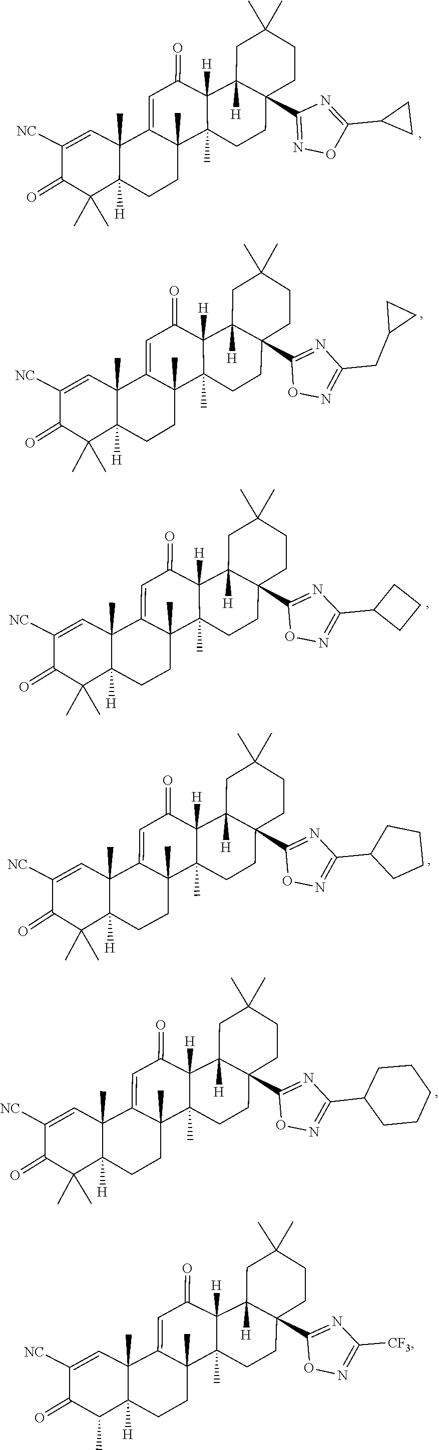

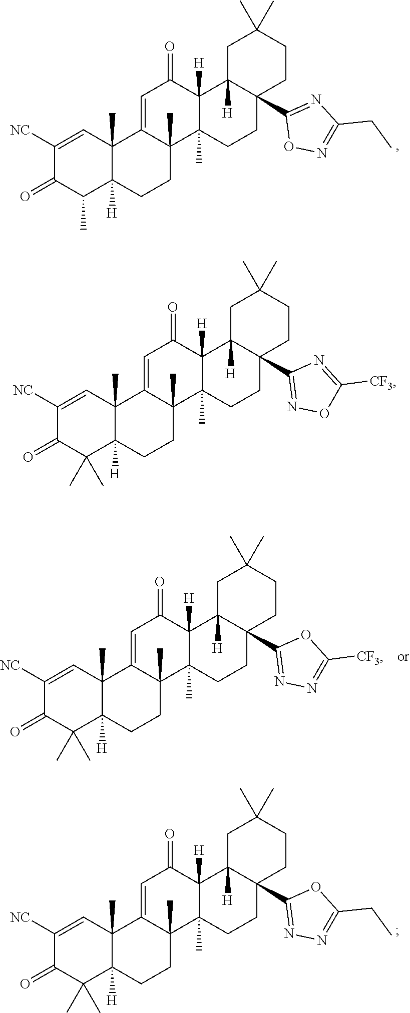

or a pharmaceutically acceptable salt of any of these formulas.

In yet another aspect, the present disclosure provides:
(4aR,6aS,6bR,8aS,12aS,12bR,14bS)-8a-(5-(2-methoxyethyl)-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(2-(2-methoxyethyl)-2H-tetrazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(2-methoxyethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-8a-(5-methyl-1,2,4-oxadiazol-3-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-8a-(2-methyl-2H-tetrazol-5-yl)-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-3,13-dioxo-8a-(3-(trifluoromethyl)-1,2,4-oxadiazol-5-yl)-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-ethyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-ethyl-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-3,13-dioxo-8a-(3-propyl-1,2,4-oxadiazol-5-yl)-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-isopropyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(tert-butyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-cyclopropyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(5-cyclopropyl-1,2,4-oxadiazol-3-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-(cyclopropylmethyl)-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-cyclobutyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-cyclopentyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-8a-(3-cyclohexyl-1,2,4-oxadiazol-5-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-4,6a,6b,11,11,14b-hexamethyl-3,13-dioxo-8a-(3-(trifluoromethyl)-1,2,4-oxadiazol-5-yl)-3,4,4a,5,6,6a,6b,7,8,8a,9,10,11,12,12a,12b,13,14b-octadecahydropicene-2-carbonitrile;
(4S,4aS,6aS,6bR,8aS,12aS,12bR,14bR)-8a-(3-ethyl-1,2,4-oxadiazol-5-yl)-4,6a,6b,11,11,14b-hexamethyl-3,13-dioxo-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-3,13-dioxo-8a-(5-(trifluoromethyl)-1,2,4-oxadiazol-3-yl)-3,4,4a,5,6,6a,6b,7,8,8a,9,10,11,12,12a,12b,13,14b-octadecahydropicene-2-carbonitrile;
(4aR,6aS,6bR,8aS,12aS,12bR,14bS)-4,4,6a,6b,11,11,14b-heptamethyl-3,13-dioxo-8a-(5-(trifluoromethyl)-1,3,4-oxadiazol-2-yl)-3,4,4a,5,6,6a,6b,7,8,8a,9,10,11,12,12a,12b,13,14b-octadecahydropicene-2-carbonitrile; or
(4aR,6aS,6bR,8aS,12aS,12bR,14bS)-8a-(5-ethyl-1,3,4-oxadiazol-2-yl)-4,4,6a,6b,11,11,14b-heptamethyl-3,13-dioxo-3,4,4a,5,6,6a,6b,7,8,8a,9,10,11,12,12a,12b,13,14b-octadecahydropicene-2-carbonitrile.

In still another aspect, the present disclosure provides pharmaceutical compositions comprising a compound of the present disclosure and an excipient. In some embodiments, the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical composition is formulated for oral administration. In other embodiments, the pharmaceutical composition is formulated for administration via injection. In some embodiments, the pharmaceutical composition is formulated for intraarterial administration, intramuscular administration, intraperitoneal administration, or intravenous administration. In some embodiments, the pharmaceutical composition is formulated for administration topically. In some embodiments, the pharmaceutical composition is formulated for topical administration to the skin or to the eye. In some embodiments, the pharmaceutical composition is formulated as a unit dose.

In another aspect, the present disclosure provides methods of treating or preventing a disease or disorder in a patient in need thereof comprising administering to the patient a pharmaceutically effective amount of a compound or composition of the present disclosure. In some embodiments, the patient is a mammal, such as a human. In some embodiments, the disease or disorder is a condition associated with inflammation and/or oxidative stress. In some embodiments, the disease or disorder is cancer. In some embodiments, the disease or disorder is a cardiovascular disease, such as atherosclerosis. In some embodiments, the disease or disorder is an autoimmune disease, such as Crohn's disease, rheumatoid arthritis, lupus, or psoriasis. In some embodiments, the disease or disorder is a neurodegenerative disease, such as Alzheimer's disease, Parkinson's disease, amyotrophic lateral sclerosis, or Huntington's disease. In some embodiments, the disease or disorder is chronic kidney disease, diabetes, mucositis, inflammatory bowel disease, dermatitis, sepsis, ischemia-reperfusion injury (including complications from sickle cell anemia), influenza osteoarthritis, osteoporosis, pancreatitis, asthma, chronic obstructive pulmonary disease, cystic fibrosis, idiopathic pulmonary fibrosis, multiple sclerosis, muscular dystrophy, cachexia, or graft-versus-host disease. In some embodiments, the disease or disorder is an eye disease, such as uveitis, glaucoma, macular degeneration, or retinopathy. In some embodiments, the disease or disorder is neuropsychiatric, such as schizophrenia, depression, bipolar disorder, epilepsy, post-traumatic stress disorder, attention deficit disorder, autism, or anorexia nervosa. In some embodiments, the disease or disorder is associated with mitochondrial dysfunction, such as Friedreich's ataxia. In some embodiments, the disease or disorder is chronic pain. In some embodiments, the disease or disorder is neuropathic pain.

In still another aspect, the present disclosure provides methods of inhibiting nitric oxide production comprising administering to a patient in need thereof an amount of a compound or composition of the present disclosure sufficient to cause inhibition of IFN-γ-induced nitric oxide production in one or more cells of the patient.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows CYP3A4 inhibition in human liver microsomes at 1 μM, with each sample, containing 0.1 mg/mL human liver microsomes, 5 μM midazolam as substrate, and 1 μM of test compound, was incubated at 37° C. for 10 min. For additional details, see Example 3.

### DESCRIPTION OF ILLUSTRATIVE EMBODIMENTS

Disclosed herein are new compounds and compositions with antioxidant and/or anti-inflammatory properties, methods for their manufacture, and methods for their use, including for the treatment and/or prevention of disease.

### I. COMPOUNDS OF THE PRESENT INVENTION

The compounds of the present invention (also referred to as “synthetic triterpenoid derivatives provided herein,” “compounds of the present disclosure” or “compounds disclosed herein”) are shown, for example, above, in the summary of the invention section, the Examples below, Table 1, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

TABLE 1
Examples of Synthetic Triterpenoid Derivatives Provided Herein

| Compound | |
|---|---|
| ID | Structural Formula |
| T1 | 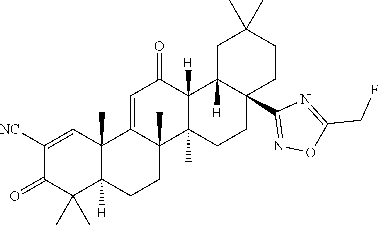 |
| T2 | 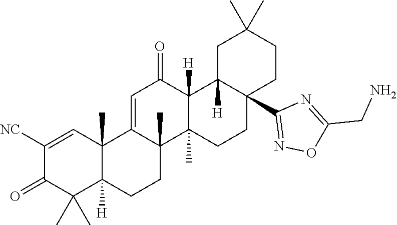 |
| T3 | 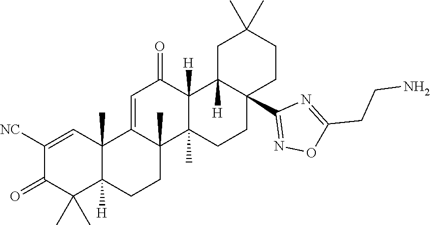 |
| T4 | 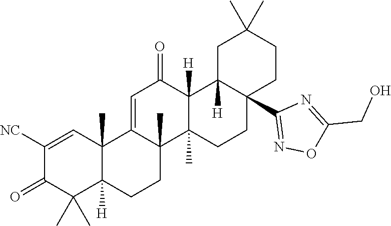 |
| T5 | 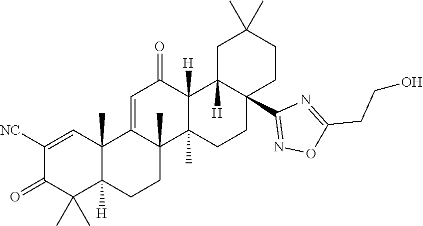 |
| T6 | 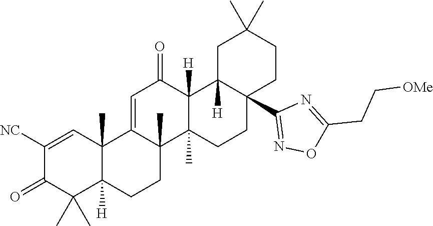 |
| T7 | 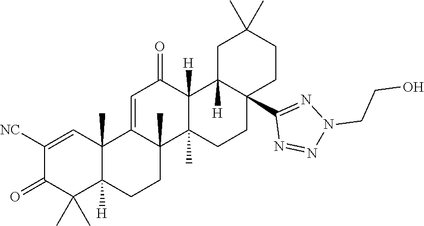 |
| T8 | 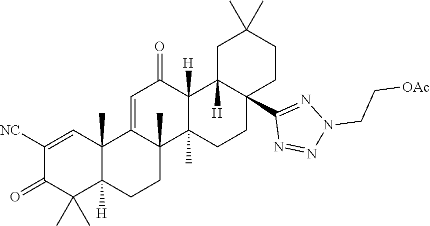 |
| T9 | 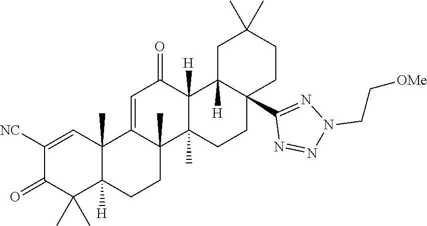 |
| T10 | 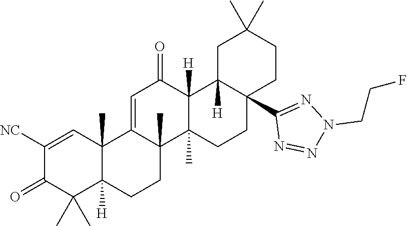 |
| T11 | 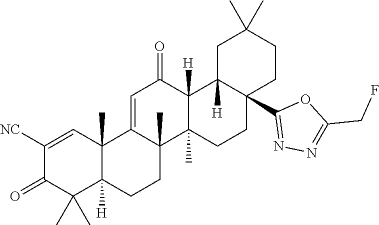 |
| T12 | 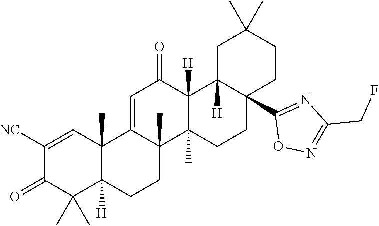 |
| T13 | 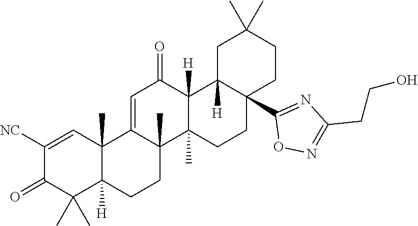 |
| T14 | 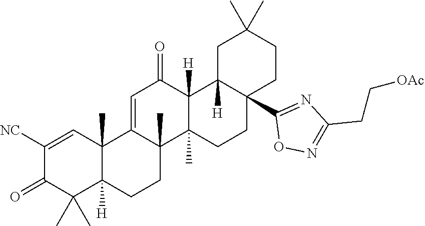 |
| T15 | 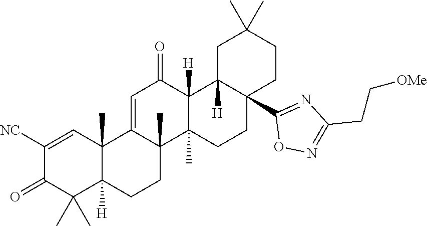 |
| T16 | 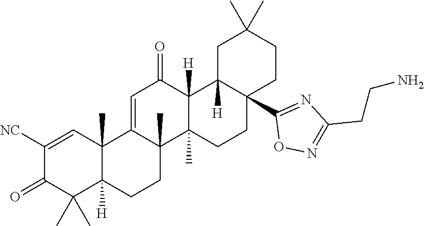 |
| T17 | 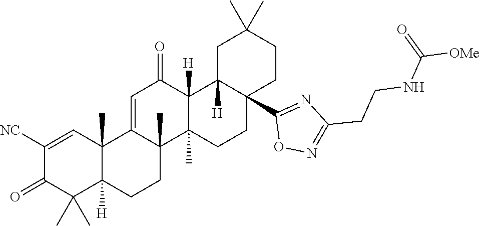 |
| T18 | 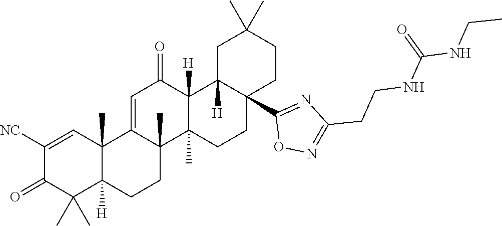 |
| T19 | 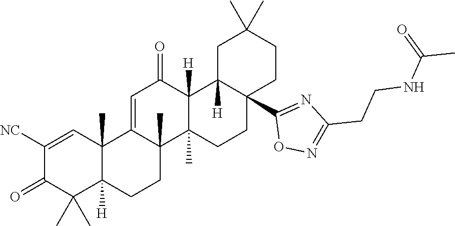 |
| T20 | 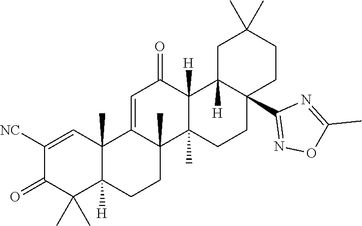 |
| T21 | 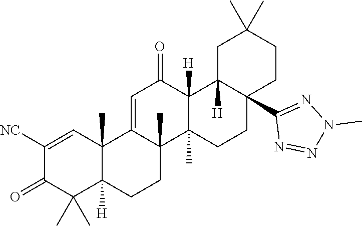 |
| T22 | 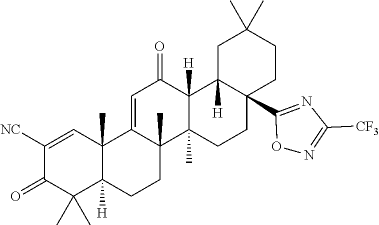 |
| T23 | 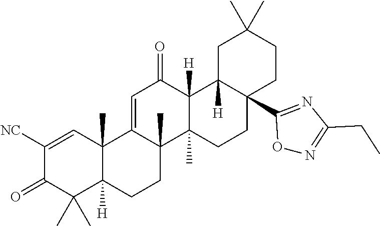 |
| T24 | 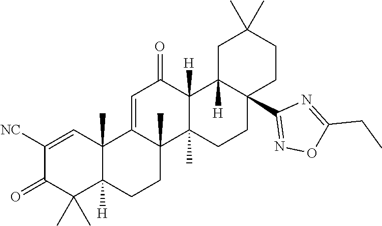 |
| T25 | 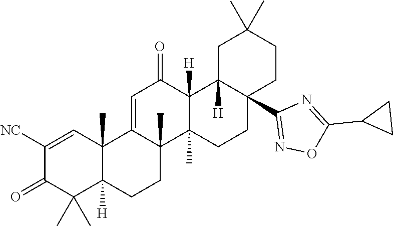 |
| T26 | 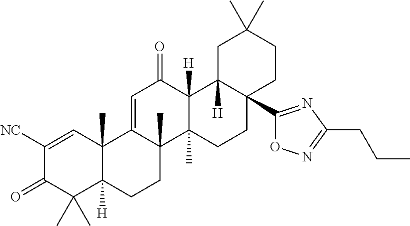 |
| T27 | 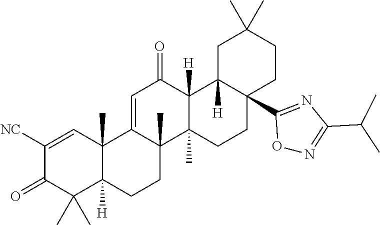 |
| T28 | 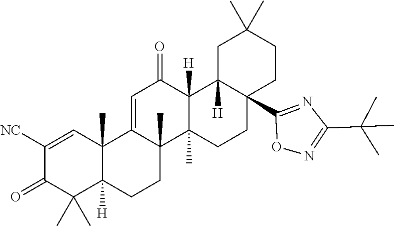 |
| T29 | 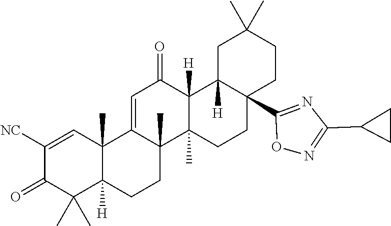 |
| T30 | 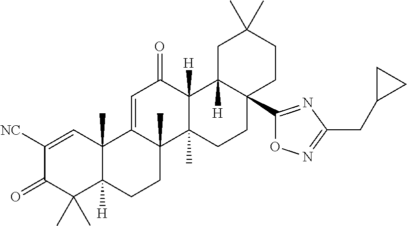 |
| T31 | 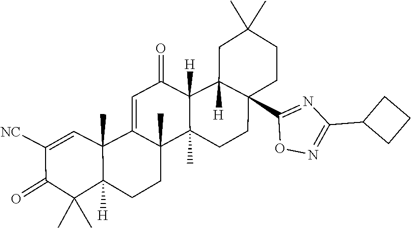 |
| T32 | 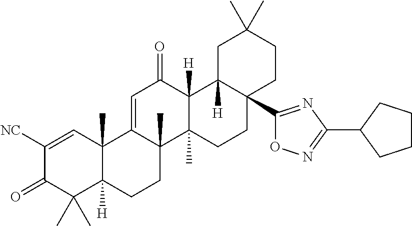 |
| T33 | 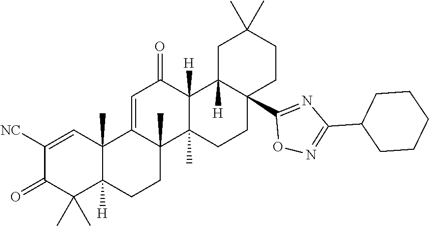 |
| T34 | 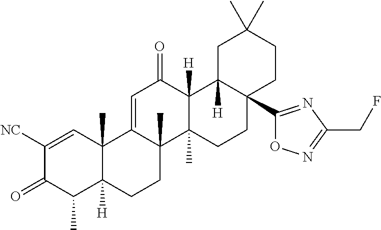 |
| T35 | 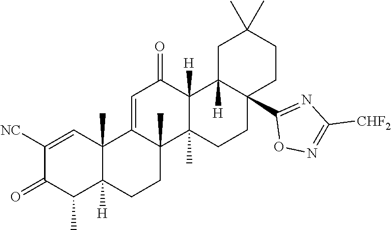 |
| T36 | 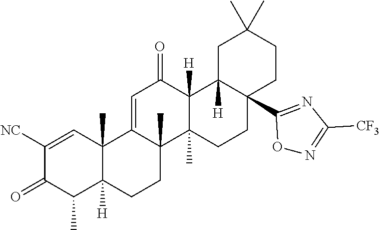 |
| T37 | 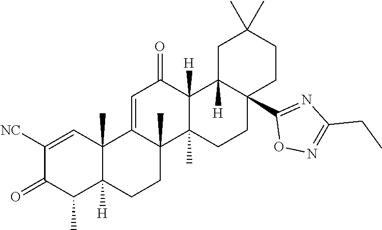 |
| T38 | 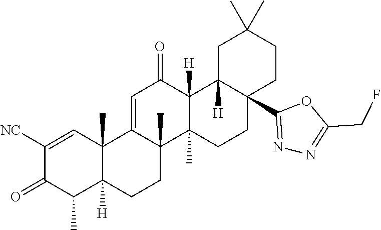 |
| T39 | 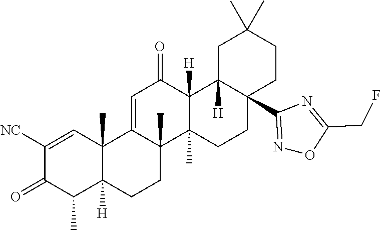 |
| T40 | 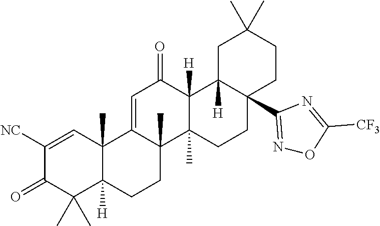 |
| T41 | 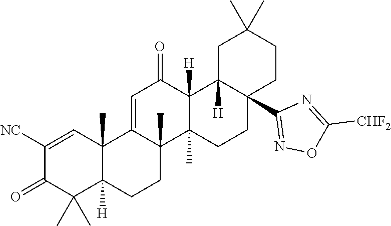 |
| T42 | 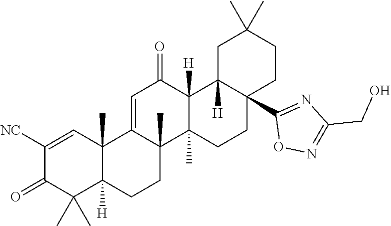 |
| T43 | 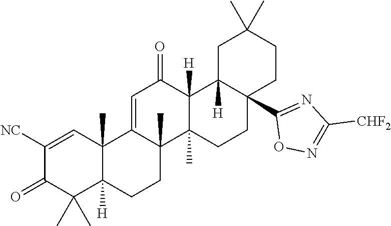 |
| T44 | 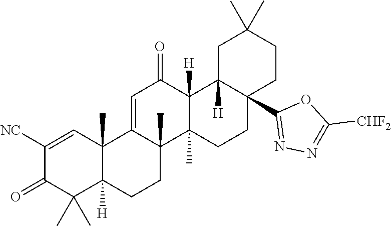 |
| T45 | 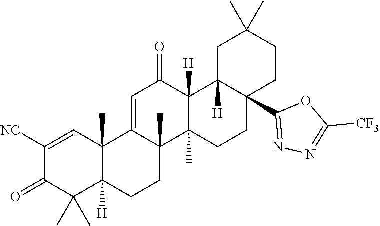 |
| T46 | 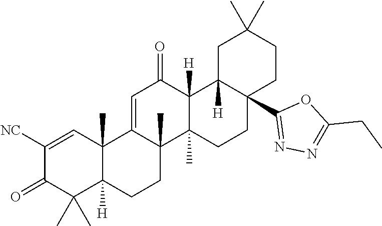 |

All the compounds of the present invention may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present invention have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

Compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated.

Compounds may occur as racemates and racemic mixtures, single enantiomers, diastereomeric mixtures and individual diastereomers. In some embodiments, a single diastereomer is obtained. The chiral centers of the compounds of the present invention can have the S or the R configuration. In some embodiments, the present compounds may contain two or more atoms which have a defined stereochemical orientation.

Chemical formulas used to represent compounds of the present invention will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present invention function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the invention contemplates prodrugs of compounds of the present invention as well as methods of delivering prodrugs. Prodrugs of the compounds employed in the invention may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present invention exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### II. BIOLOGICAL ACTIVITY

Assay results for the suppression of IFNγ-induced NO production are shown for several of the compounds of the present invention in Table 2 and Table 3 in Example 2. Table 2 presents the results compared to those of bardoxolone methyl (RTA 402, CDDO-Me). Table 3 present the results compared to comparison compounds CC1, CC2, and CC3. Details regarding this assay are provided in the Examples section below.

In some embodiments, the synthetic triterpenoid derivatives provided herein that were substituted with polar substituents at the C17 heteroaryl groups exhibited improved nitric oxide inhibition compared with compounds lacking such substituents, such as those disclosed in U.S. Pat. No. 9,512,094, which is incorporated herein by reference. For example, the IC₅₀ value for fluoro-substituted T12 is 36% lower (1.27 nM vs. 1.98 nM) than the corresponding unsubstituted compound, CC2 (TX63501; U.S. Pat. No. 9,512,094). In another example, the IC₅₀ values for hydroxy-substituted T13 and acetoxy-substituted T14 were 88% and 90% lower (0.56 nM and 0.48 nM, respectively vs. 4.85 nM) than the corresponding unsubstituted compound, T23. In another example, the IC₅₀ values for fluoro-substituted T1, amino-substituted T2, and hydroxy-substituted T4 are 67%, 70%, and 52% lower (1.24 nM, 1.15 nM, and 1.82 nM, respectively vs. 3.79 nM) than the corresponding unsubstituted compound, T20. Similarly, amino-substituted T3 and hydroxy-substituted T5 are 72% and 83% lower (2.60 nM and 1.57 nM, respectively vs. 9.21 nM) than the corresponding unsubstituted compound, T24. In another example, the IC₅₀ value for fluoro-substituted T11 is 52% lower (0.98 nM vs. 2.05 nM) than the corresponding unsubstituted compound, CC1 (TX63384; U.S. Pat. No. 9,512,094). In yet another example, the IC₅₀ value for fluoro-substituted T34 is 31% lower (0.93 nM vs. 1.34 nM) than the corresponding unsubstituted compound, CC3 (TX63787; U.S. Pat. No. 9,290,536). In some embodiments, complete replacement of all the hydrogens with polar substituents results in a decrease in nitric oxide inhibitory activity. Compare the trifluoromethyl derivative T22 (23.95 nM) with the monofluoromethyl derivative T12 (1.27 nM).

In some embodiments, the compounds of the present disclosure exhibit reduced inhibition of cytochrome P450 3A4 (CYP3A4) relative to known compounds. CYP3A4 is an important enzyme in the body, which oxidizes small foreign organic molecules (xenobiotics), such as toxins or drugs, so that they can be removed from the body. Modulation of CYP3A4 may amplify or weaken the action of drugs that are modified by CYP3A4. Inhibition of CYP3A4 may have negative side effects (e.g. reduced drug clearance, amplification of the action of the drug, and/or increase the probability of drug-drug interactions) and may complicate dosing. Therefore, drugs that do not inhibit CYP3A4 are often more desirable.

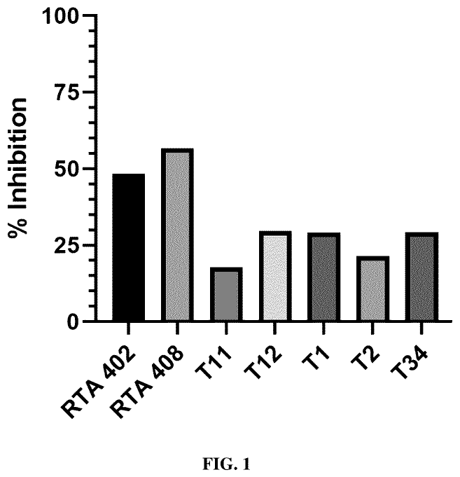

Assay results for the inhibition of CYP3A4 are shown for several of the compounds of the present disclosure in Tables 4-7 in Example 3. In some embodiments, the synthetic triterpenoid derivatives provided herein that were substituted with polar substituents at the C17 heteroaryl groups exhibited reduced CYP3A4 inhibition compared with compounds lacking such substituents, such as those disclosed in U.S. Pat. Nos. 9,512,094 and 9,290,536, both of which are incorporated herein by reference. For example, the CYP3A4 inhibition values for fluoro-substituted T1, amino-substituted T2, and fluoro-substituted T12 were 36%, 53%, and 35% lower (29.1%, 21.4%, and 29.7% inhibition, respectively, vs. 45.8% inhibition) than the corresponding unsubstituted compound, CC2 (TX63501; U.S. Pat. No. 9,512,094). In another example, the CYP3A4 inhibition for fluoro-substituted T11 was 15% lower (17.7% inhibition vs. 20.7% inhibition) than the corresponding unsubstituted compound, CC1 (TX63384; U.S. Pat. No. 9,512,094). In another example, the CYP3A4 inhibition for fluoro-substituted T34 was 22% lower (29.4% inhibition vs. 37.7% inhibition) than the corresponding unsubstituted compound, CC3 (TX63787; U.S. Pat. No. 9,290,536). In addition, each of T1, T2, T11, T12, and T34 exhibited reduced CYP3A4 inhibition compared with historical data for RTA 402 and RTA 408 performed under comparable conditions (FIG. 1).

### III. DISEASES ASSOCIATED WITH INFLAMMATION AND/OR OXIDATIVE STRESS

Inflammation is a biological process that provides resistance to infectious or parasitic organisms and the repair of damaged tissue. Inflammation is commonly characterized by localized vasodilation, redness, swelling, and pain, the recruitment of leukocytes to the site of infection or injury, production of inflammatory cytokines such as TNF-α and IL-1, and production of reactive oxygen or nitrogen species such as hydrogen peroxide, superoxide and peroxynitrite. In later stages of inflammation, tissue remodeling, angiogenesis, and scar formation (fibrosis) may occur as part of the wound healing process. Under normal circumstances, the inflammatory response is regulated and temporary and is resolved in an orchestrated fashion once the infection or injury has been dealt with adequately. However, acute inflammation can become excessive and life-threatening if regulatory mechanisms fail. Alternatively, inflammation can become chronic and cause cumulative tissue damage or systemic complications. Based at least on the evidence presented above, the compounds of the present disclosure may be used in the treatment or prevention of inflammation or diseases associated with inflammation.

Many serious and intractable human diseases involve dysregulation of inflammatory processes, including diseases such as cancer, atherosclerosis, and diabetes, which were not traditionally viewed as inflammatory conditions. In the case of cancer, the inflammatory processes are associated with tumor formation, progression, metastasis, and resistance to therapy. Atherosclerosis, long viewed as a disorder of lipid metabolism, is now understood to be primarily an inflammatory condition, with activated macrophages playing an important role in the formation and eventual rupture of atherosclerotic plaques. Activation of inflammatory signaling pathways has also been shown to play a role in the development of insulin resistance, as well as in the peripheral tissue damage associated with diabetic hyperglycemia. Excessive production of reactive oxygen species and reactive nitrogen species such as superoxide, hydrogen peroxide, nitric oxide, and peroxynitrite is a hallmark of inflammatory conditions. Evidence of dysregulated peroxynitrite production has been reported in a wide variety of diseases (Szabo et al., 2007; Schulz et al., 2008; Forstermann, 2006; Pall, 2007). Aging-related diseases such as dementia, muscle wasting, cardiovascular diseases, neurodegenerative diseases, and arthritis frequently involve chronic inflammation and oxidative stress as major contributing factors. In some embodiments, the compounds provided herein may be used to treat and/or prevent aging-related diseases, such as dementia, muscle wasting, cardiovascular diseases, neurodegenerative diseases, or arthritis.

Autoimmune diseases such as rheumatoid arthritis, lupus, psoriasis, and multiple sclerosis involve inappropriate and chronic activation of inflammatory processes in affected tissues, arising from dysfunction of self vs. non-self-recognition and response mechanisms in the immune system. In some embodiments, the compounds provided herein may be used in the treatment and/or prevention of autoimmune diseases, such as rheumatoid arthritis, lupus, psoriasis, or multiple sclerosis. In neurodegenerative diseases such as Alzheimer's and Parkinson's diseases, neural damage is correlated with activation of microglia and elevated levels of pro-inflammatory proteins such as inducible nitric oxide synthase (iNOS). In some embodiments, the compounds provided herein may be used in the treatment and/or prevention of neurodegenerative diseases, such as Alzheimer's disease or Parkinson's disease. Chronic organ failure such as renal failure, heart failure, liver failure, and chronic obstructive pulmonary disease is closely associated with the presence of chronic oxidative stress and inflammation, leading to the development of fibrosis and eventual loss of organ function. In some embodiments, the compounds provided herein may be used in the treatment and/or prevention of chronic organ failure, such as renal failure, heart failure, liver failure, or chronic obstructive pulmonary disease. Oxidative stress in vascular endothelial cells, which line major and minor blood vessels, can lead to endothelial dysfunction and is believed to be an important contributing factor in the development of systemic cardiovascular disease, complications of diabetes, chronic kidney disease and other forms of organ failure, and a number of other aging-related diseases including degenerative diseases of the central nervous system and the retina. In some embodiments, the compounds provided herein may be used in the treatment and/or prevention of systemic cardiovascular disease, complications of diabetes, chronic kidney disease and other forms of organ failure, and a number of other aging-related diseases including degenerative diseases of the central nervous system and the retina.

Many other disorders involve oxidative stress and inflammation in affected tissues, including inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury; chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, and eating disorders such as anorexia nervosa. In some embodiments, the compounds provided herein may be used in the treatment and/or prevention of inflammatory bowel disease; inflammatory skin diseases; mucositis related to radiation therapy and chemotherapy; eye diseases such as uveitis, glaucoma, macular degeneration, and various forms of retinopathy; transplant failure and rejection; ischemia-reperfusion injury (including complications of sickle cell anemia); chronic pain; degenerative conditions of the bones and joints including osteoarthritis and osteoporosis; asthma and cystic fibrosis; seizure disorders; and neuropsychiatric conditions including schizophrenia, depression, bipolar disorder, post-traumatic stress disorder, attention deficit disorders, autism-spectrum disorders, or eating disorders such as anorexia nervosa. Dysregulation of inflammatory signaling pathways is believed to be a major factor in the pathology of muscle wasting diseases including muscular dystrophy and various forms of cachexia. In some embodiments, the compounds provided herein may be used in the treatment and/or prevention of muscle wasting diseases, such as muscular dystrophy and various forms of cachexia.

A variety of life-threatening acute disorders also involve dysregulated inflammatory signaling, including acute organ failure involving the pancreas, kidneys, liver, or lungs, myocardial infarction or acute coronary syndrome, stroke, septic shock, trauma, severe burns, and anaphylaxis.

Many complications of infectious diseases also involve dysregulation of inflammatory responses. Although an inflammatory response can kill invading pathogens, an excessive inflammatory response can also be quite destructive and in some cases can be a primary source of damage in infected tissues. Furthermore, an excessive inflammatory response can also lead to systemic complications due to overproduction of inflammatory cytokines such as TNF-α and IL-1. This is believed to be a significant factor in mortality arising from severe influenza, severe acute respiratory syndrome due to infection with coronaviruses including SARS-CoV-2, which causes COVID-19, and other viruses causing upper respiratory disease, and sepsis. In some embodiments, the compounds provided herein may be used in the treatment and/or prevention of influenza, severe acute respiratory syndrome due to infection with coronaviruses including SARS-Cov-2 or other viruses causing upper respiratory disease, or sepsis.

The aberrant or excessive expression of either iNOS or cyclooxygenase-2 (COX-2) has been implicated in the pathogenesis of many disease processes. For example, it is clear that NO is a potent mutagen (Tamir and Tannebaum, 1996), and that nitric oxide can also activate COX-2 (Salvemini et al., 1994). Furthermore, there is a marked increase in iNOS in rat colon tumors induced by the carcinogen, azoxymethane (Takahashi et al., 1997). A series of synthetic triterpenoid analogs of oleanolic acid have been shown to be powerful inhibitors of cellular inflammatory processes, such as the induction by IFN-γ of inducible nitric oxide synthase (iNOS) and of COX-2 in mouse macrophages. See Honda et al. (2000a); Honda et al. (2000b), and Honda et al. (2002), which are all incorporated herein by reference. Elevated brain tissue levels of iNOS have also been associated with Alzheimer's disease (Sporn et al., 1996). In some embodiments, the compounds provided herein may be used in the treatment and/or prevention of Alzheimer's disease.

In one aspect, compounds disclosed herein are characterized by their ability to inhibit the production of nitric oxide in macrophage-derived RAW 264.7 cells induced by exposure to γ-interferon. They are further characterized by their ability to induce the expression of antioxidant proteins such as NQO1 and reduce the expression of pro-inflammatory proteins such as COX-2 and inducible nitric oxide synthase (iNOS). These properties are relevant to the treatment of a wide array of diseases and disorders involving oxidative stress and dysregulation of inflammatory processes including cancer, complications from localized or total-body exposure to ionizing radiation, mucositis resulting from radiation therapy or chemotherapy, autoimmune diseases, cardiovascular diseases including atherosclerosis, ischemia-reperfusion injury (including complications of sickle cell anemia), acute and chronic organ failure including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases including osteoarthritis and osteoporosis, inflammatory bowel diseases, dermatitis and other skin diseases, sepsis, burns, seizure disorders, and neuropsychiatric disorders. In some embodiments, the compounds provided herein may be used in the treatment and/or prevention of cancer, complications from localized or total-body exposure to ionizing radiation, mucositis resulting from radiation therapy or chemotherapy, autoimmune diseases, cardiovascular diseases including atherosclerosis, ischemia-reperfusion injury (including complications of sickle cell anemia), acute and chronic organ failure including renal failure and heart failure, respiratory diseases, diabetes and complications of diabetes, severe allergies, transplant rejection, graft-versus-host disease, neurodegenerative diseases, diseases of the eye and retina, acute and chronic pain, degenerative bone diseases including osteoarthritis and osteoporosis, inflammatory bowel diseases, dermatitis and other skin diseases, sepsis, burns, seizure disorders, or neuropsychiatric disorders.

Without being bound by theory, the activation of the antioxidant/anti-inflammatory Keap1/Nrf2/ARE pathway is believed to be implicated in both the anti-inflammatory and anti-carcinogenic properties of the compounds disclosed herein.

In another aspect, compounds disclosed herein may be used for treating a subject having a condition caused by elevated levels of oxidative stress in one or more tissues. Oxidative stress results from abnormally high or prolonged levels of reactive oxygen species such as superoxide, hydrogen peroxide, nitric oxide, and peroxynitrite (formed by the reaction of nitric oxide and superoxide). The oxidative stress may be accompanied by either acute or chronic inflammation. The oxidative stress may be caused by mitochondrial dysfunction, by activation of immune cells such as macrophages and neutrophils, by acute exposure to an external agent such as ionizing radiation or a cytotoxic chemotherapy agent (e.g., doxorubicin), by trauma or other acute tissue injury, by ischemia/reperfusion, by poor circulation or anemia, by localized or systemic hypoxia or hyperoxia, by elevated levels of inflammatory cytokines and other inflammation-related proteins, and/or by other abnormal physiological states such as hyperglycemia or hypoglycemia. In some embodiments, the compounds provided herein may be used in the treatment and/or prevention of mitochondrial dysfunction and disorders associated therewith.

In animal models of many such conditions, stimulating expression of inducible heme oxygenase (HO-1), a target gene of the Nrf2 pathway, has been shown to have a significant therapeutic effect including models of myocardial infarction, renal failure, transplant failure and rejection, stroke, cardiovascular disease, and autoimmune disease (e.g., Sacerdoti et al., 2005; Abraham & Kappas, 2005; Bach, 2006; Araujo et al., 2003; Liu et al., 2006; Ishikawa et al., 2001; Kruger et al., 2006; Satoh et al., 2006; Zhou et al., 2005; Morse and Choi, 2005; Morse and Choi, 2002). This enzyme breaks free heme down into iron, carbon monoxide (CO), and biliverdin (which is subsequently converted to the potent antioxidant molecule, bilirubin). Carbon monoxide has been shown to have signaling functions, and biliverdin reductase, the enzyme that catalyzes the conversion of biliverdin to bilirubin, has been shown to function as a dual-specificity kinase as well as modulate HO-1 expression (Motterlini & Foresti, 2017; Florczyk et al., 2008).

In another aspect, compounds of the present disclosure may be used in preventing or treating tissue damage or organ failure, acute and chronic, resulting from oxidative stress exacerbated by inflammation. Examples of diseases that fall in this category include: heart failure, liver failure, transplant failure and rejection, renal failure, pancreatitis, fibrotic lung diseases (cystic fibrosis, COPD, and idiopathic pulmonary fibrosis, among others), diabetes (including complications), atherosclerosis, ischemia-reperfusion injury including complications of sickle cell anemia, glaucoma, stroke, autoimmune disease, autism, macular degeneration, and muscular dystrophy. For example, in the case of autism, studies suggest that increased oxidative stress in the central nervous system may contribute to the development of the disorder (Chauhan and Chauhan, 2006).

Evidence also links oxidative stress and inflammation to the development and pathology of many other disorders of the central nervous system, including psychiatric disorders such as psychosis, major depression, post-traumatic stress disorder (PTSD), and bipolar disorder; seizure disorders such as epilepsy; pain and sensory syndromes such as migraine, neuropathic pain or tinnitus; and behavioral syndromes such as the attention deficit disorders. See, e.g., Dickerson et al., 2007; Hanson et al., 2005; Kendall-Tackett, 2007; Lencz et al., 2007; Dudhgaonkar et al., 2006; Lee et al., 2007; Morris et al., 2002; Ruster et al., 2005; McIver et al., 2005; Sarchielli et al., 2006; Kawakami et al., 2006; Ross et al., 2003, which are all incorporated by reference herein. For example, elevated levels of inflammatory cytokines, including TNF, interferon-γ, and IL-6, are associated with major mental illness (Dickerson et al., 2007). Microglial activation has also been linked to major mental illness. Therefore, downregulating inflammatory cytokines and inhibiting excessive activation of microglia could be beneficial in patients with schizophrenia, major depression, bipolar disorder, autism-spectrum disorders, and other neuropsychiatric disorders. In some embodiments, the compounds provided herein may be used in the treatment and/or prevention of disorders of the central nervous system, including psychiatric disorders such as psychosis, major depression, post-traumatic stress disorder (PTSD), and bipolar disorder; seizure disorders such as epilepsy; pain and sensory syndromes such as migraine, neuropathic pain or tinnitus; or behavioral syndromes such as the attention deficit disorders.

Accordingly, in pathologies involving oxidative stress alone or oxidative stress exacerbated by inflammation, treatment may comprise administering to a subject a therapeutically effective amount of a compound of the present disclosure, such as those described above or throughout this specification. Treatment may be administered preventively, in advance of a predictable state of oxidative stress (e.g., organ transplantation or the administration of radiation therapy to a cancer patient), or it may be administered therapeutically in settings involving established oxidative stress and inflammation.

The compounds disclosed herein may be generally applied to the treatment of inflammatory conditions, such as sepsis, dermatitis, autoimmune disease and osteoarthritis. In one aspect, the compounds of the present disclosure may be used to treat inflammatory pain and/or neuropathic pain, for example, by inducing Nrf2 and/or inhibiting NF-κB.

In some embodiments, the compounds disclosed herein may be used in the treatment and prevention of diseases such as cancer, inflammation, Alzheimer's disease, Parkinson's disease, multiple sclerosis, autism, amyotrophic lateral sclerosis, Huntington's disease, autoimmune diseases such as rheumatoid arthritis, lupus, Crohn's disease and psoriasis, inflammatory bowel disease, all other diseases whose pathogenesis is believed to involve excessive production of either nitric oxide or prostaglandins, and pathologies involving oxidative stress alone or oxidative stress exacerbated by inflammation.

Another aspect of inflammation is the production of inflammatory prostaglandins such as prostaglandin E. These molecules promote vasodilation, plasma extravasation, localized pain, elevated temperature, and other symptoms of inflammation. The inducible form of the enzyme COX-2 is associated with their production, and high levels of COX-2 are found in inflamed tissues. Consequently, inhibition of COX-2 may relieve many symptoms of inflammation, and a number of important anti-inflammatory drugs (e.g., ibuprofen and celecoxib) act by inhibiting COX-2 activity. Recent research, however, has demonstrated that a class of cyclopentenone prostaglandins (cyPGs) (e.g., 15-deoxy prostaglandin J2, a.k.a. PGJ2) plays a role in stimulating the orchestrated resolution of inflammation (e.g., Rajakariar et al., 2007). COX-2 is also associated with the production of cyclopentenone prostaglandins. Consequently, inhibition of COX-2 may interfere with the full resolution of inflammation, potentially promoting the persistence of activated immune cells in tissues and leading to chronic, “smoldering” inflammation. This effect may be responsible for the increased incidence of cardiovascular disease in patients using selective COX-2 inhibitors for long periods of time.

In one aspect, the compounds disclosed herein may be used to control the production of pro-inflammatory cytokines within the cell by selectively activating regulatory cysteine residues (RCRs) on proteins that regulate the activity of redox-sensitive transcription factors. Activation of RCRs by cyPGs has been shown to initiate a pro-resolution program in which the activity of the antioxidant and cytoprotective transcription factor Nrf2 is potently induced and the activities of the pro-oxidant and pro-inflammatory transcription factors NF-κB and the STATs are suppressed. In some embodiments, this increases the production of antioxidant and reductive molecules (NQO1, HO-1, SOD1, γ-GCS) and decreases oxidative stress and the production of pro-oxidant and pro-inflammatory molecules (iNOS, COX-2, TNF-α). In some embodiments, the compounds of the present disclosure may cause the cells that host the inflammatory event to revert to a non-inflammatory state by promoting the resolution of inflammation and limiting excessive tissue damage to the host.

### IV. PHARMACEUTICAL FORMULATIONS AND ROUTES OF ADMINISTRATION

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

HED (mg/kg)=Animal dose (mg/kg)×(Animal Kₘ/Human Kₘ)

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### V. COMBINATION THERAPY

In addition to being used as a monotherapy, the compounds of the present disclosure may also be used in combination therapies. In some embodiments, compounds of the present disclosure may be combined with one or more agents that promote proper folding or assembly of CFTR (correctors) or that enhance the function of CFTR (potentiators). For example, combinations can include a compound of the invention combined with one or more correctors, one or more potentiators, a corrector and a potentiator. In other examples, the combination includes an amplifier either with just one of the compounds of the present invention or in combination with a compound of the present invention and the above described combinations of correctors and potentiators.

In some embodiments, there are provided combination therapies, wherein a compound disclosed herein is combined with another CF treatment, for example, a compound designed to improve function of CFTR that has reached the cell membrane and is capable of at least partial function. Such compounds are known as CFTR potentiators, and the first disease-specific therapy for CF, ivacaftor, has been clinically demonstrated to improve CFTR function in patients with several of the significant mutations. Compounds that prevent misfolding of CFTR are known as correctors. In some embodiments the compounds of the present invention may be used to function as correctors. The enhanced efficacy for the treatment of CF from combining two correctors, or a corrector with a potentiator, is well understood in the art and such combinations have been approved for marketing or are currently being studied in clinical trials. Combinations of three agents are also being studied in clinical trials. It is recognized that that polytherapies are or may soon become the standard of care. In some embodiments, other classes of CFTR modulators, such as “amplifiers” that increase the steady-state levels of CFTR, may become available and may also be used as part of a polytherapy.

Other potential combinations will be apparent to the skilled practitioner. In some embodiments, effective combination therapy is achieved with a single composition or pharmacological formulation that includes multiple agents, or with two or more distinct compositions or formulations, administered at the same time, wherein one composition includes a compound of the present disclosure, and the other(s) include(s) the additional agent(s), formulated together or separately. Alternatively, in other embodiments, the therapy precedes or follows the other agent treatment by intervals ranging from minutes to months.

### VI. DEFINITIONS

The definitions below supersede any conflicting definition in any reference that is incorporated by reference herein. The fact that certain terms are defined, however, should not be considered as indicative that any term that is undefined is indefinite. Rather, all terms used are believed to describe the present disclosure in terms such that one of ordinary skill can appreciate the scope and practice the present disclosure.

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

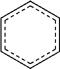

covers, for example,

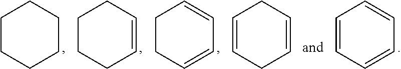

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g., [symbol] for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper. For example, the following two depictions are equivalent:

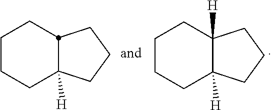 [SMILES: C1CC[C@@H]2CCC[C@H]2C1 ; C1CC[C@@H]2CCC[C@H]2C1]

When a variable is depicted as a “floating group” on a ring system, for example, the group “R” in the formula:

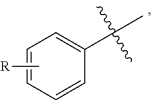

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

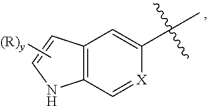

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn−n′” defines both the minimum (n) and maximum number (n′) of carbon atoms in the group. Thus, “alkyl_{(C≤10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C1-4-alkyl”, “C1-4-alkyl”, “alkyl(ci-4)”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

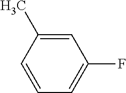

is also taken to refer to

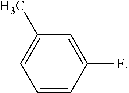

Aromatic compounds may also be depicted using a circle to represent the delocalized nature of the electrons in the fully conjugated cyclic π system, two non-limiting examples of which are shown below;

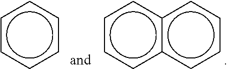

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, Pr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H-R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

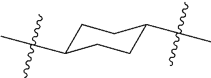 [SMILES: *C1CCC(*)CC1]

is a non-limiting example of cycloalkanediyl group. A “cycloalkane” refers to the class of compounds having the formula H-R, wherein R is cycloalkyl as this term is defined above.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group. The term “heterocycloalkanediyl” refers to a divalent cyclic group, with two carbon atoms, two nitrogen atoms, or one carbon atom and one nitrogen atom as the two points of attachment, said atoms forming part of one or more ring structure(s) wherein at least one of the ring atoms of the non-aromatic ring structure(s) is nitrogen, oxygen or sulfur, and wherein the divalent group consists of no atoms other than carbon, hydrogen, nitrogen, oxygen and sulfur. If more than one ring is present, the rings are fused. As used herein, the term heterocycloalkanediyl does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkanediyl groups include:

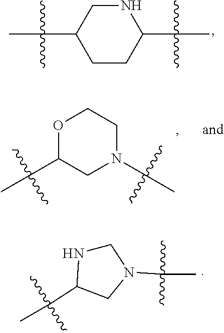

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H-R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H-R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused.

Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

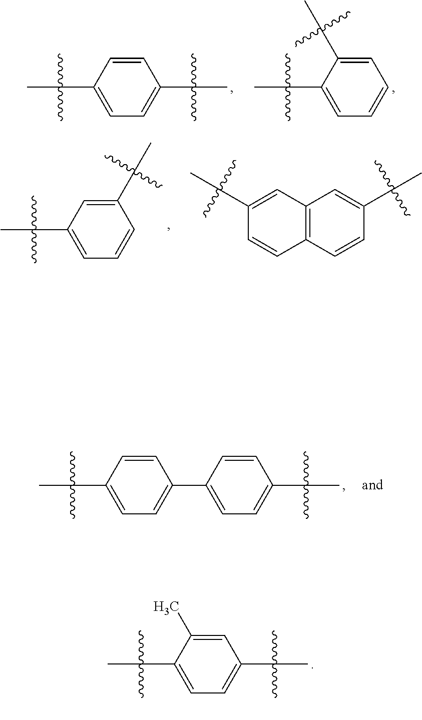

An “arene” refers to the class of compounds having the formula H-R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H-R, wherein R is heteroaryl. Pyridine and quinoline are non-limiting examples of heteroarenes. The term “heteroarenediyl” refers to a divalent aromatic group, with two aromatic carbon atoms, two aromatic nitrogen atoms, or one aromatic carbon atom and one aromatic nitrogen atom as the two points of attachment, said atoms forming part of one or more aromatic ring structures, each with three to eight ring atoms, wherein at least one of the ring atoms of the aromatic ring structure(s) is nitrogen, oxygen or sulfur, and wherein the divalent group consists of no atoms other than carbon, hydrogen, aromatic nitrogen, aromatic oxygen and aromatic sulfur. If more than one ring is present, the rings are fused; however, the term heteroarenediyl does not preclude the presence of one or more alkyl or aryl groups (carbon number limitation permitting) attached to one or more ring atoms. Non-limiting examples of heteroarenediyl groups include:

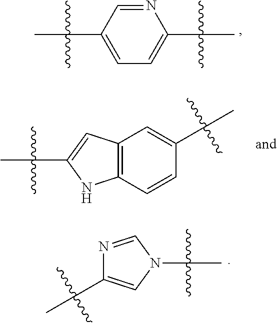

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, and “alkoxyamino” when used without the “substituted” modifier, refers to groups, defined as —NHR, in which R is cycloalkyl, alkenyl, alkynyl, aryl, aralkyl, heteroaryl, heterocycloalkyl, and alkoxy, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂C1. The term “hydroxyalkyl” is a subset of substituted alkyl, in which one or more hydrogen atom has been replaced with a hydroxy (i.e. —OH) group, such that no other atoms aside from carbon, hydrogen, and oxygen are present. The groups —CH₂OH, —CH₂CH₂OH, —CH(OH)CHOH, —CH₂CH(OH)CH₃, and —CH(OH)CH₂OH are non-limiting examples of hydroxyalkyl groups. The term “monohydroxyalkyl” is a subset of substituted alkyl, in which one hydrogen atom has been replaced with a hydroxy (i.e. —OH) group, such that no other atoms aside from carbon, hydrogen, and one oxygen are present. The groups —CH₂OH, —CH₂CH₂OH, and —CH₂CH(OH)CH₃ are non-limiting examples of monohydroxyalkyl groups. The term “fluoroalkyl” is a subset of substituted alkyl, in which one or more hydrogen atom has been replaced with a fluoro, such that no other atoms aside from carbon, hydrogen, and fluorine are present. The groups —CH₂F, —CHF₂, and —CF₃ are non-limiting examples of fluoroalkyl groups. The term “monofluoroalkyl” is a subset of substituted alkyl, in which one hydrogen atom has been replaced with a fluoro, such that no other atoms aside from carbon, hydrogen, and one fluorine are present. The groups —CH₂F, —CH₂CH₂F, and —CH₂CH(F)CH₃ are non-limiting examples of monofluoroalkyl groups. The term “aminoalkyl” is a subset of substituted alkyl, in which one or more hydrogen atom has been replaced with an amino (i.e. —NH₂) group, such that no other atoms aside from carbon, hydrogen, and nitrogen are present. The groups —CH₂NH₂, —CH(NH₂)CH₃, —CH₂CH₂NH₂, —CH₂CH(NH₂)CH₃ and —CH(NH₂)CH₂NH₂ are non-limiting examples of aminoalkyl groups. The term “monoaminoalkyl” is a subset of substituted alkyl, in which one hydrogen atom has been replaced with an amino (i.e. —NH₂) group, such that no other atoms aside from carbon, hydrogen, and one nitrogen are present. The groups —CH₂NH₂, —CH₂CH₂NH₂, and —CH₂CH(NH₂)CH₃ are non-limiting examples of monoaminoalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

When a chemical group is used with the “polar-substituted” modifier, one or more hydrogen atom has been replaced, independently at each instance, by one of the following polar substituents: —OH, —F, —NH₂, —CO₂H, —CO₂CH₃, —C(O)NH₂, —C(O)NHCH₃, —OC(O)CH₃, —NHC(O)CH₃, —NHC(O)OCH₃, —NHC(O)OCH₂CH₃, —NHC(O)NHCH₃, —NHC(O)NHCH₂CH₃, —S(O)₂OH, or —S(O)₂NH₂, provided that not every hydrogen is so replaced. Non-limiting examples of polar-substituted alkyl groups include —CH₂F, —CHF₂, —CH₂CH₂F, —CHFCH₂F, —CF₂CH₃, —CH₂OH, —CH₂CH₂OH, —CH₂CH₂CH₂NH₂, —CH₂CH₂OH, and —CH(NH₂)CH₂OH.

When a chemical group is used with the “monopolar-substituted” modifier, one and only one hydrogen atom has been replaced by one of the following polar substituents: —OH, —F, —NH₂, —CO₂H, —CO₂CH₃, —C(O)NH₂, —C(O)NHCH₃, —OC(O)CH₃, —NHC(O)CH₃, —NHC(O)OCH₃, —NHC(O)OCH₂CH₃, —NHC(O)NHCH₃, —NHC(O)NHCH₂CH₃, —S(O)₂OH, or —S(O)₂NH₂. Non-limiting examples of monopolar-substituted alkyl groups include —CH₂F, —CH₂CH₂F, —CHFCH₃, —CH₂OH, —CH₂CH₂OH, —CH(OH)CH₂OH, —CH₂NH₂, —CH₂CH₂NH₂, and —CH(NH₂)CH₃.

Some of the abbreviations used herein are as follows: Ac refers to an acetyl group (—C(O)CH₃); Boc refers to tert-butyloxycarbonyl; COPD stands for chronic obstructive pulmonary disease; COX-2 refers to cyclooxygenase-2; CYP3A4 stands for cytochrome P450 3A4; cyPGs refers to cyclopentenone prostaglandins; DBDMH refers to 1,3-Dibromo-5,5-dimethylhydantoin; DIBAL-H is diisobutylaluminium hydride; DMAP refers to 4-dimethylaminopyridine; DMF is dimethylformamide; DMSO is dimethyl sulfoxide; EDC is 1-ethyl-3-(3-dimethylaminopropyl)carbodiimide; Et₂O, diethyl ether; HO-1 refers to inducible heme oxygenase; IFNγ or IFN-γ refer to interferon-γ; IL-1 refers to the interleukin-1 family; iNOS stands for inducible nitric oxide synthase; NCS refers to N-Chlorosuccinimide; NMO refers to N-methylmorpholine N-oxide; NO stands for nitric oxide; NQO1 stands for NAD(P)H dehydrogenase (quinone 1); Nrf2 refers to nuclear factor erythroid 2-related factor 2; OA refers to oleanolic acid; Py stands for Pyridine; T3P refers to propylphosphonic anhydride; TFA is trifluoroacetic acid; TFAA stands for trifluoroacetic anhydride; THF is tetrahydrofuran; TNFα or TNF-α, tumor necrosis factor-α; TPAP is tetrapropylammonium perruthenate; Ts stands for tosyl; TsOH or p-TsOH is p-toluenesulfonic acid; 4 Å MS refers to 4 angstrom molecular sieves.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “relative IC₅₀” refers to the fold difference in potency between two compounds. It is determined by dividing the IC₅₀ value of a compound of interest by the IC₅₀ value of a benchmark compound within each experimental assay using the following equation:

$$Relative IC 50 ( Fold ) = \frac{IC 50 ( Compound of Interest )}{IC 50 ( Benchmark )}$$

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof.

In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of the present disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present disclosure. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-p-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: Synthesis and Characterization

### A. General Information

Unless otherwise stated, commercially reagents were used as received, and all reactions were run under nitrogen atmosphere. All solvents were of HPLC or ACS grade. Nuclear magnetic resonance (NMR) spectra were recorded on a Varian Inova-400 spectrometer at operating frequencies of 400 MHz (¹H NMR). Chemical shifts (6) are given in ppm relative to residual solvent (usually chloroform δ 7.26 ppm for ¹H NMR) and coupling constants (J) in Hz. Multiplicity is tabulated as s for singlet, d for doublet, t for triplet, q for quadruplet, and m for multiplet. Mass spectra were recorded on Agilent 6120 mass spectrometer. The compounds of the present disclosure may be prepared according to the methods outlined in Example 1 as well as methods known to a skilled artisan, including those disclosed in WO 2012/125488 and WO 2014/040056, both of which are incorporated by reference herein.

### B. Synthetic Routes to Compounds of the Present Disclosure

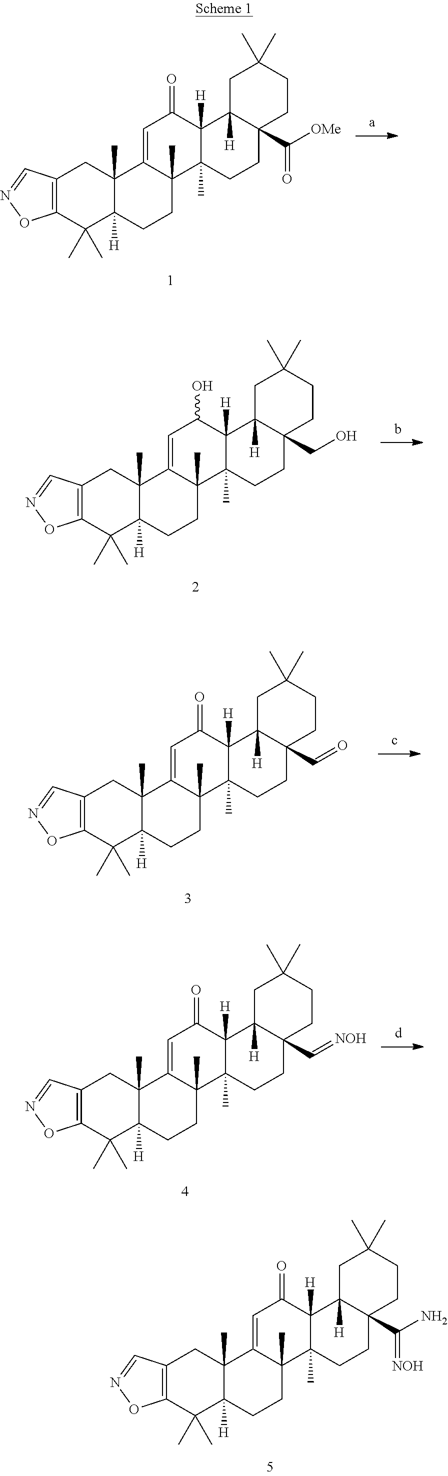

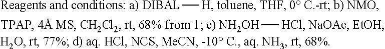

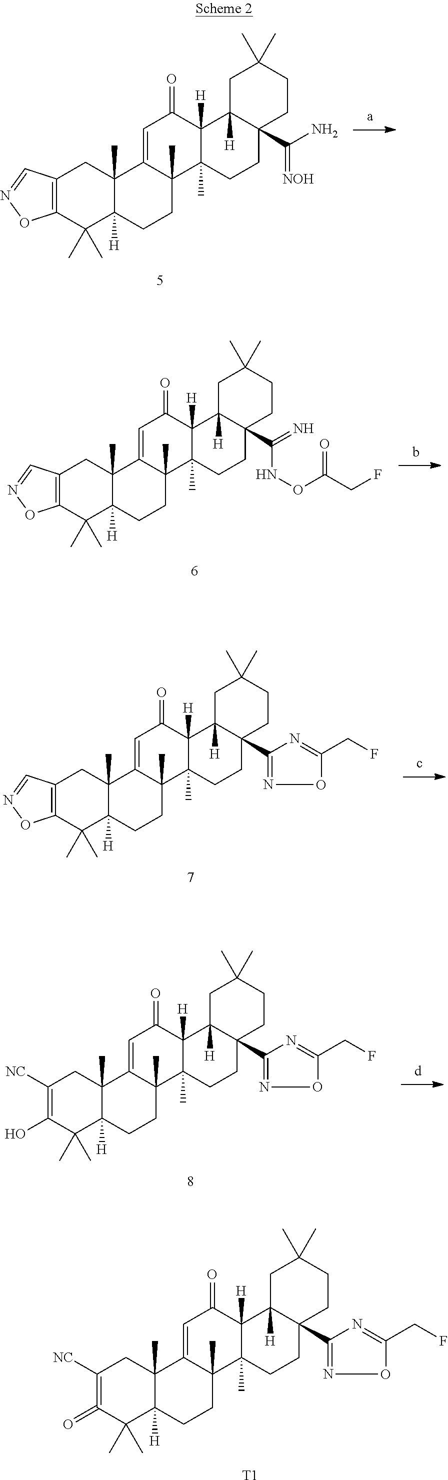

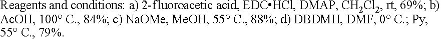

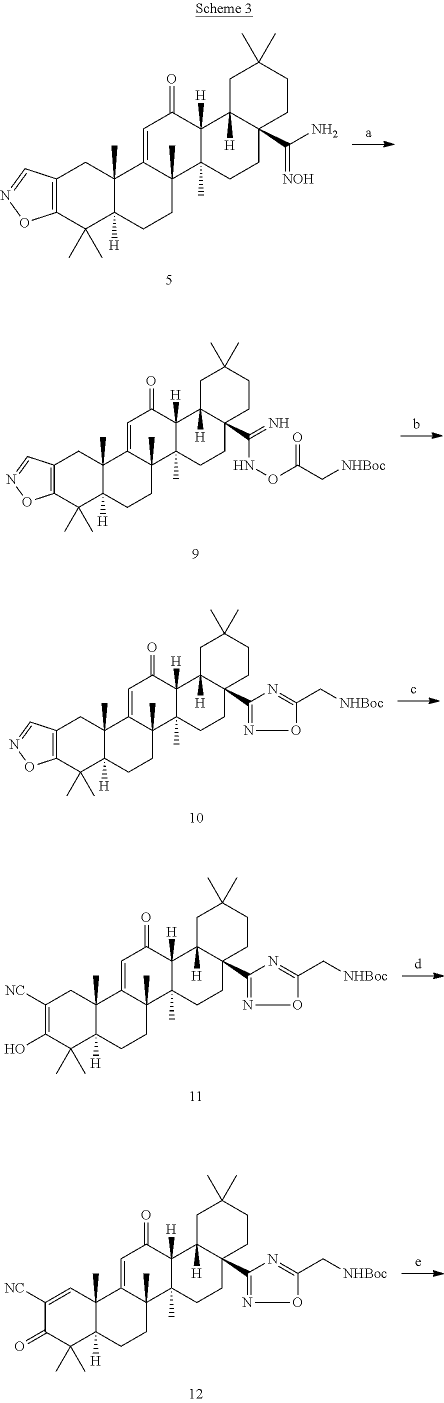

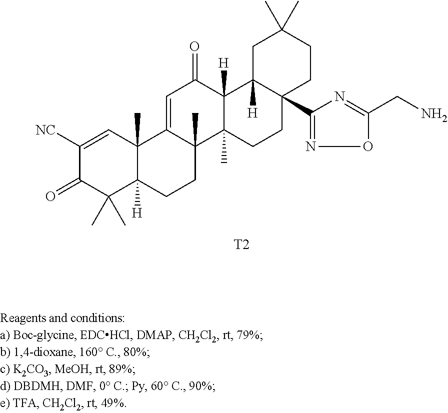

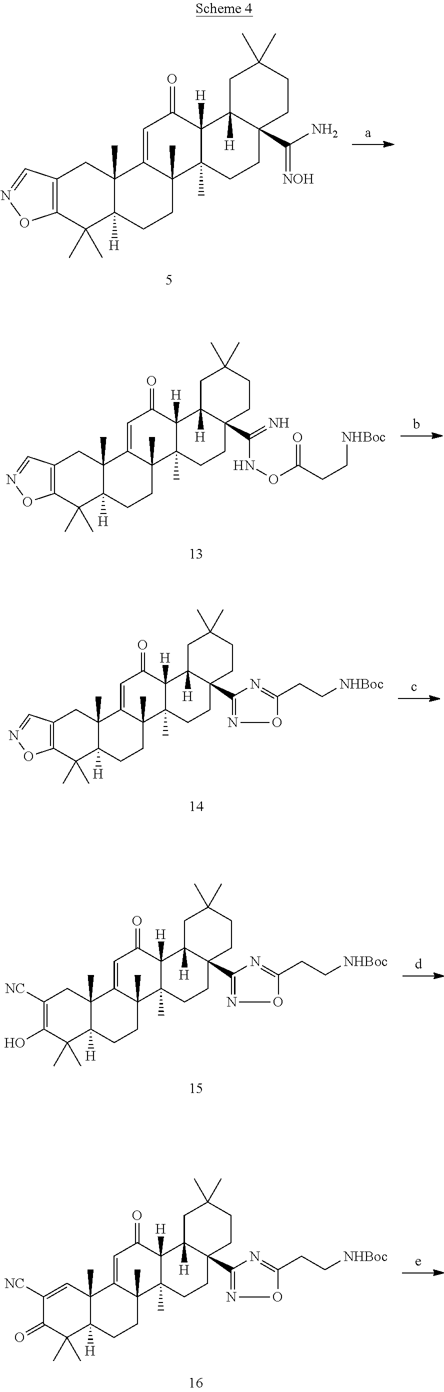

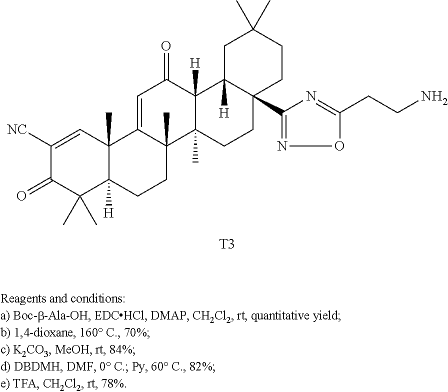

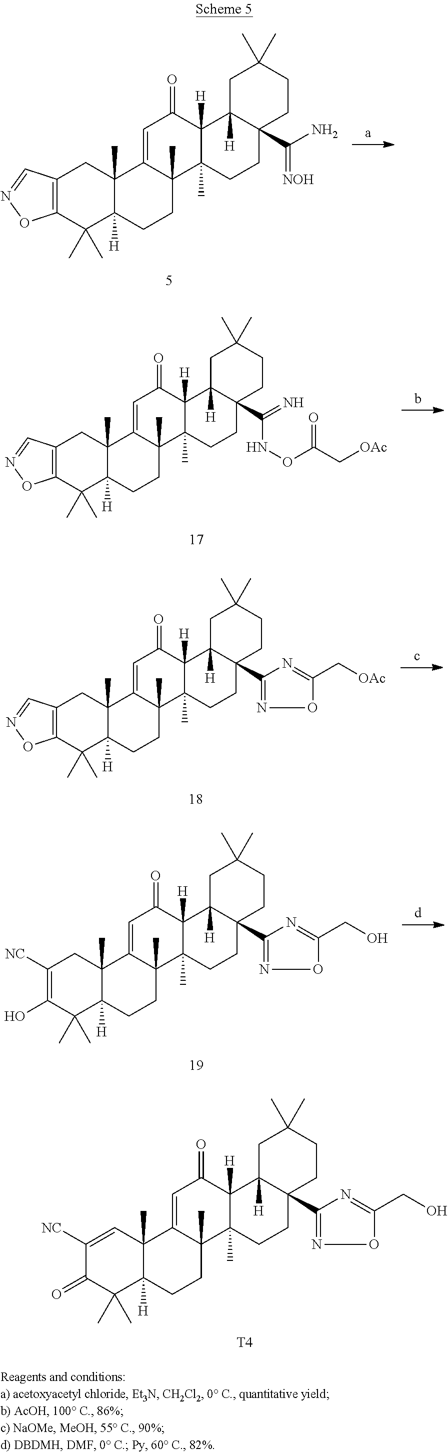

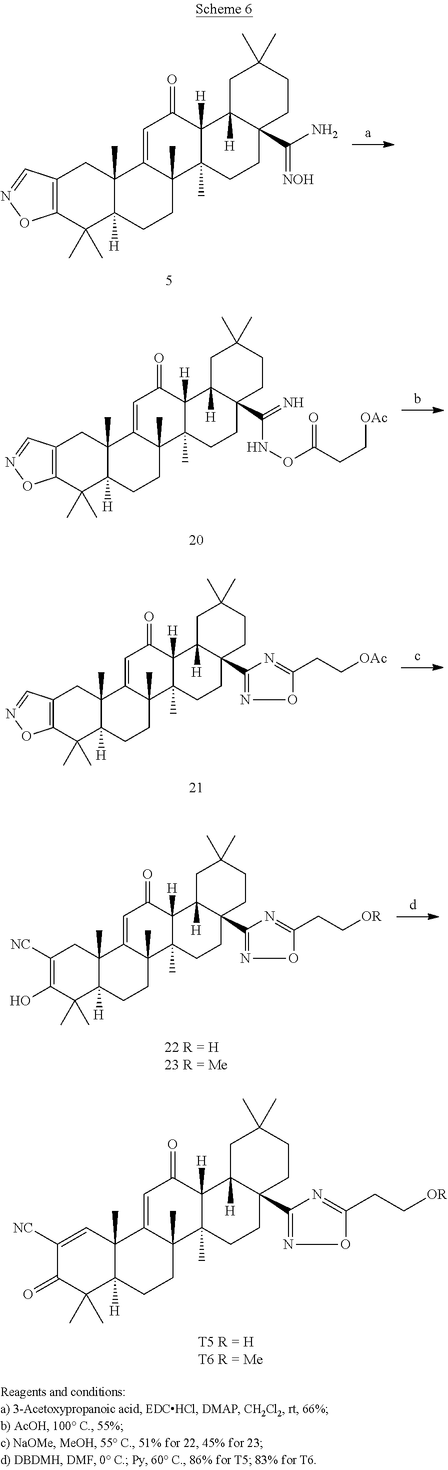

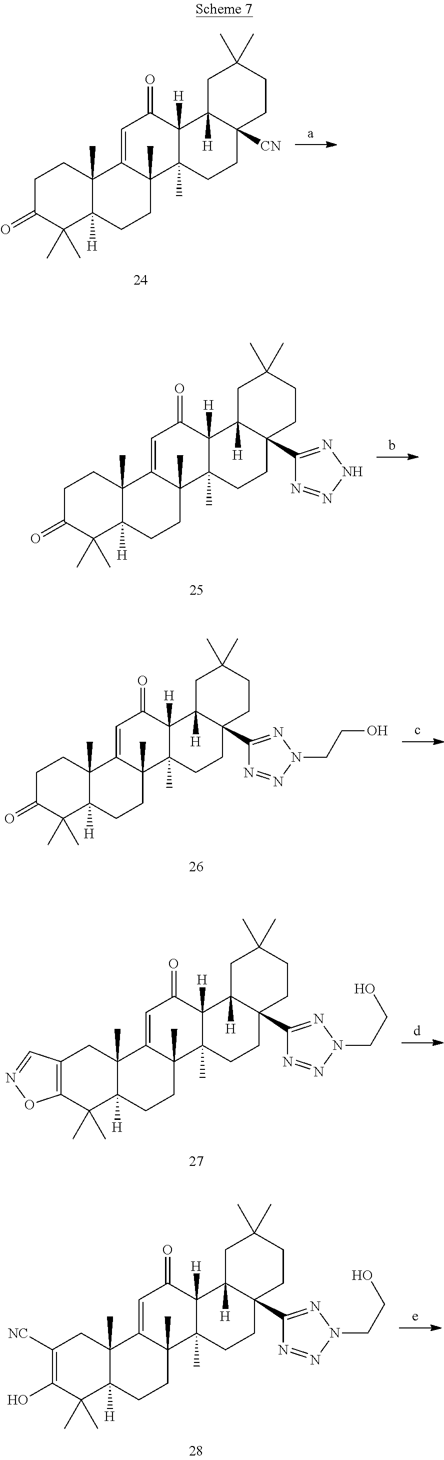

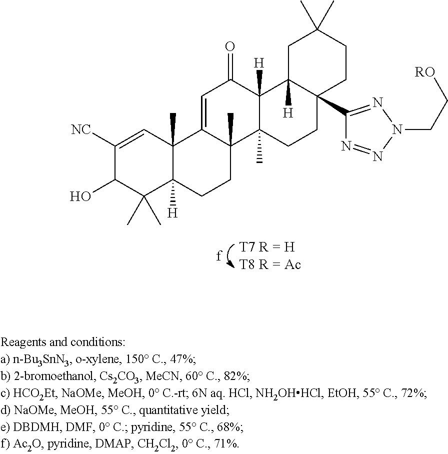

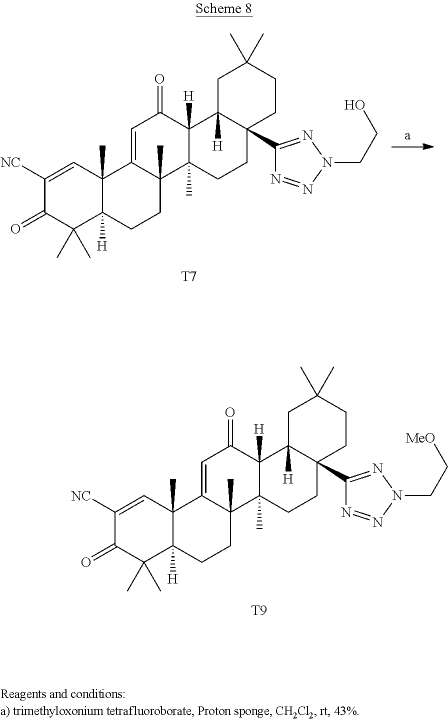

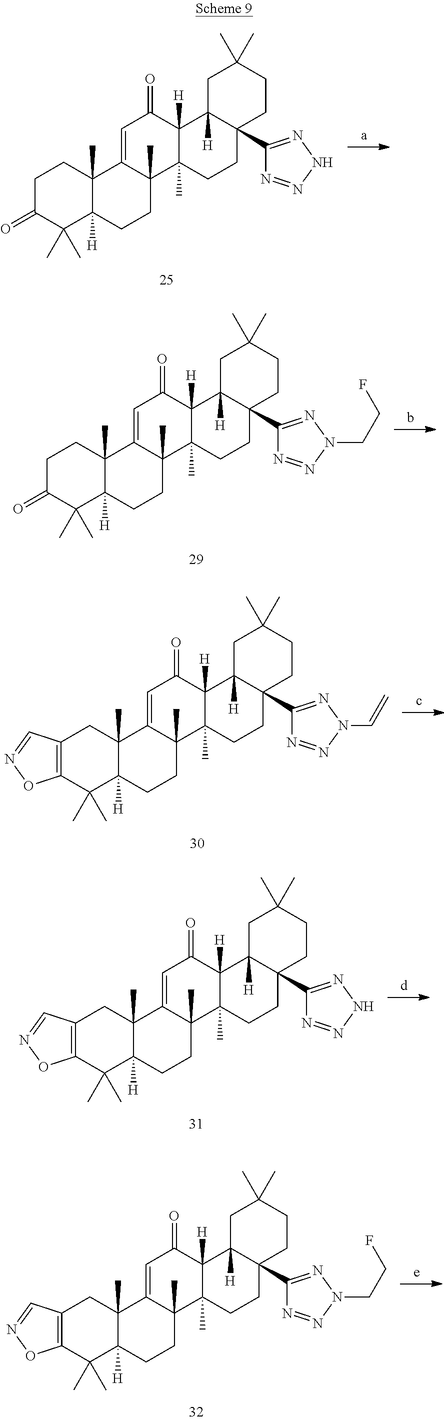

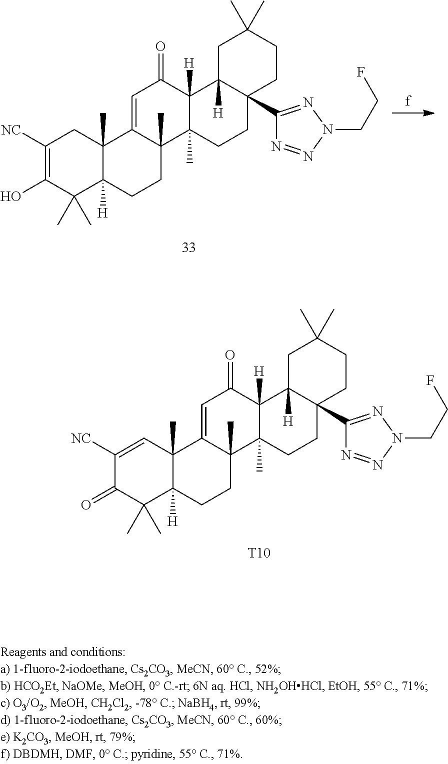

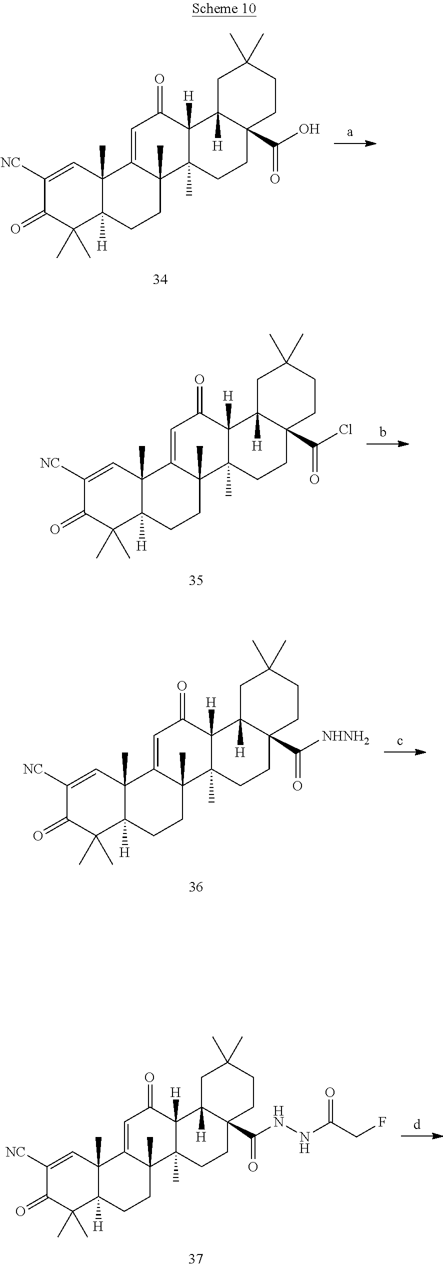

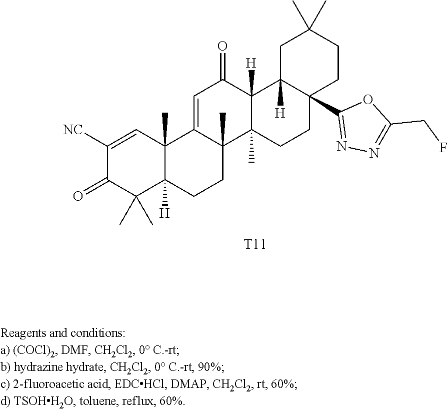

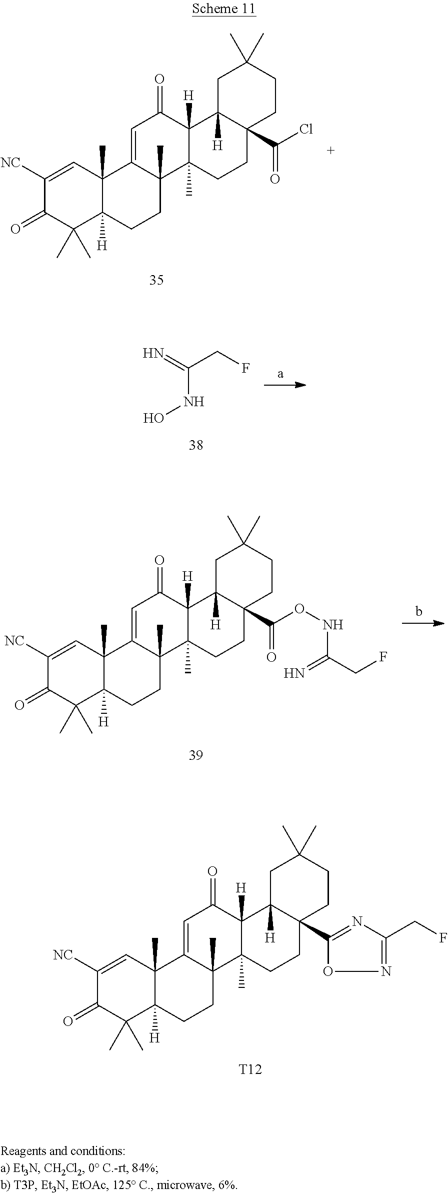

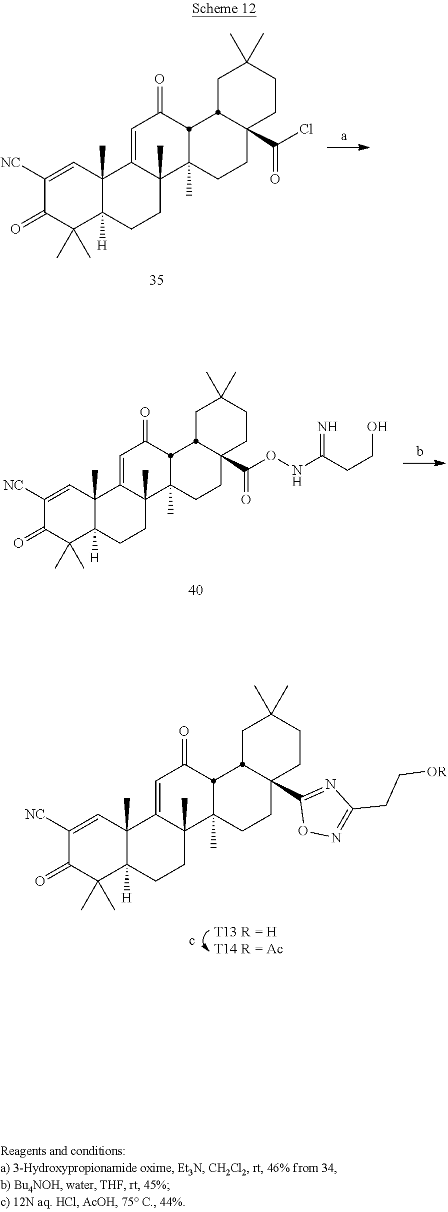

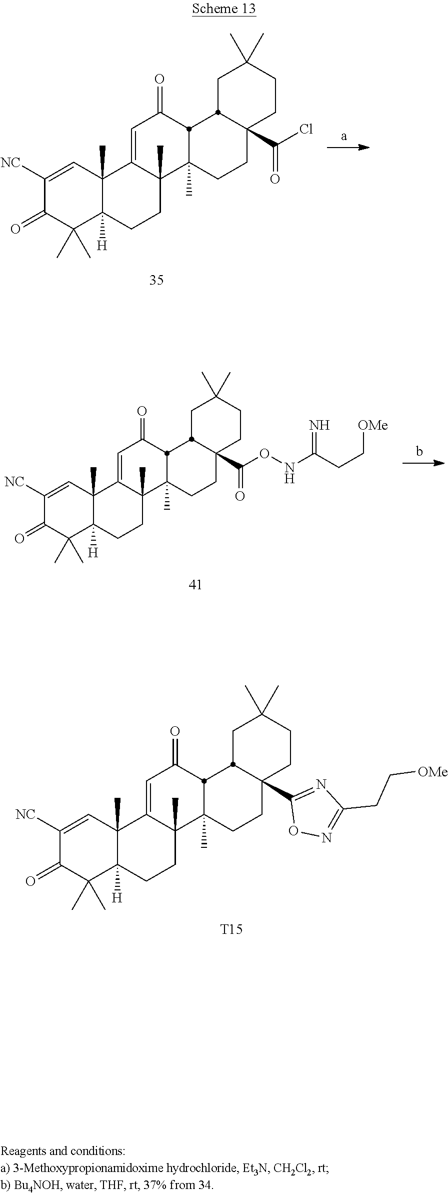

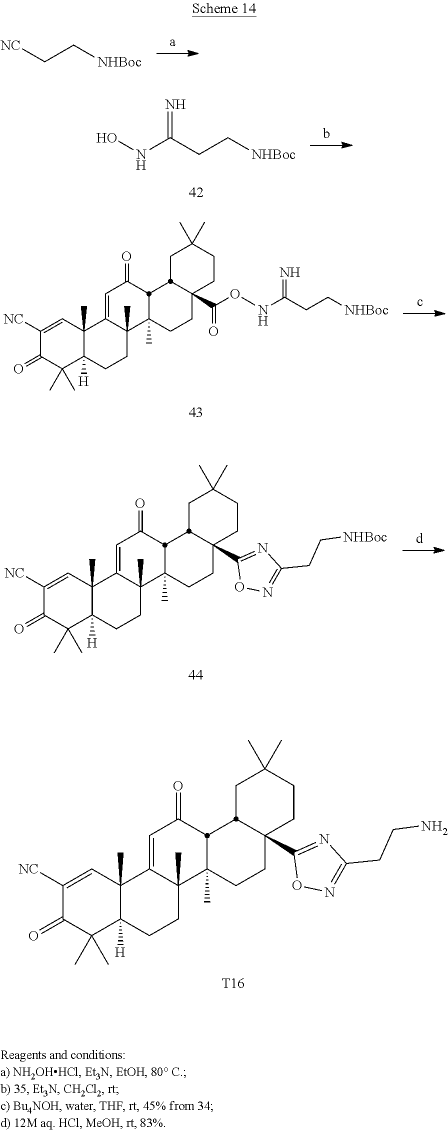

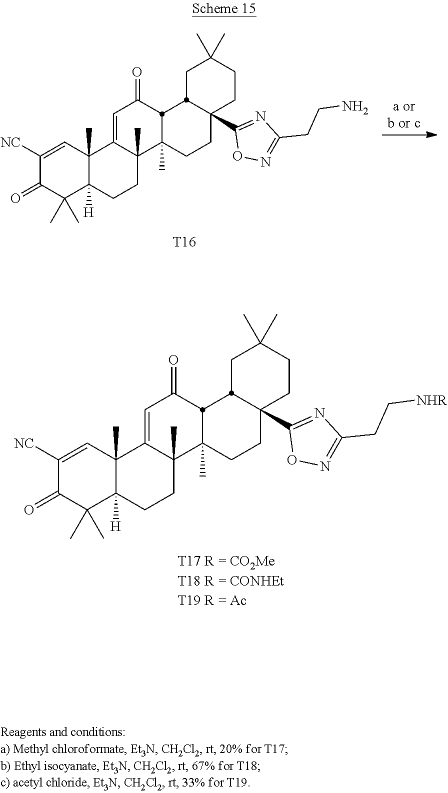

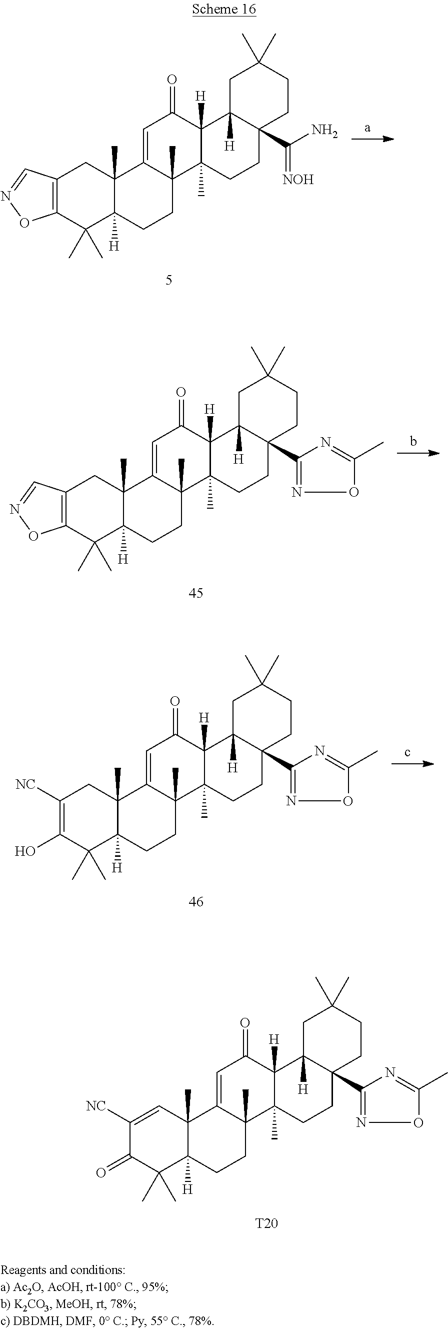

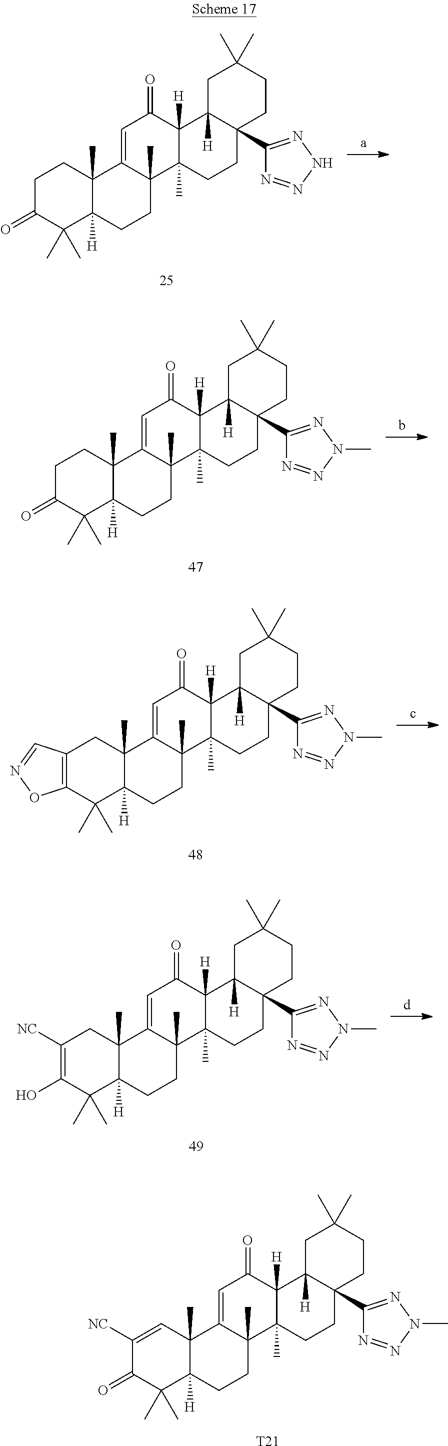

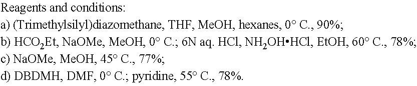

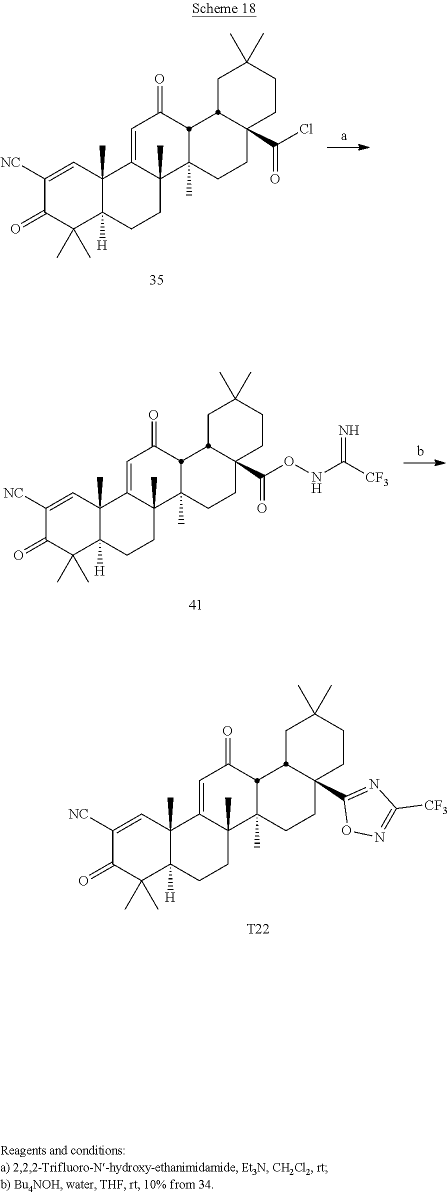

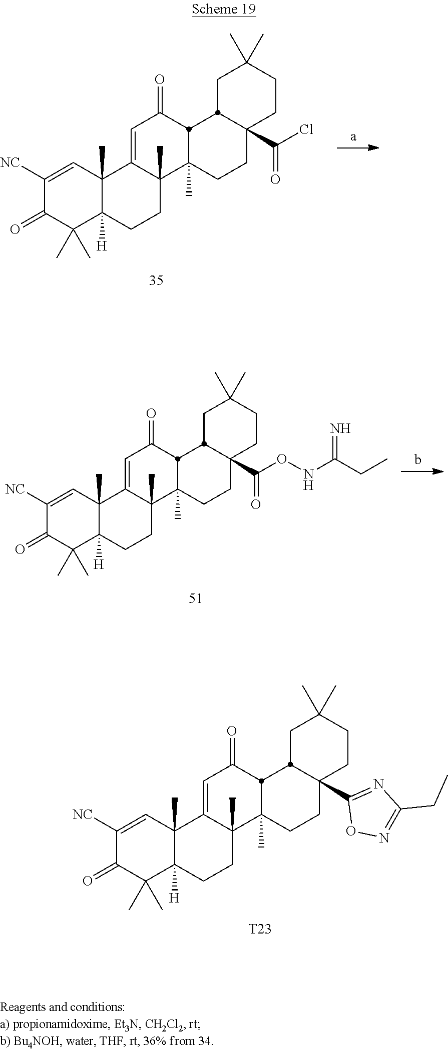

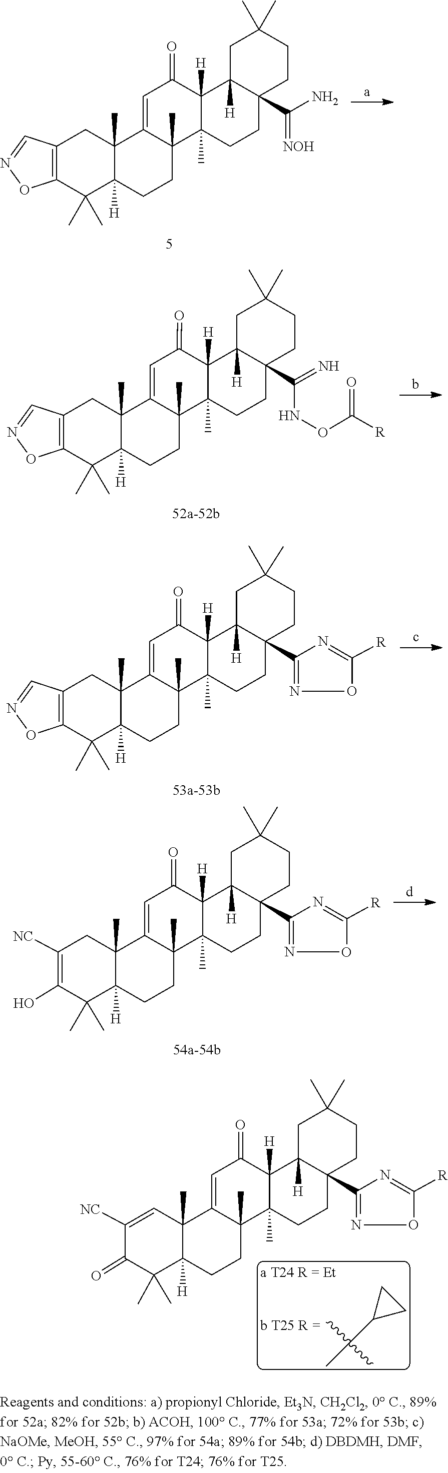

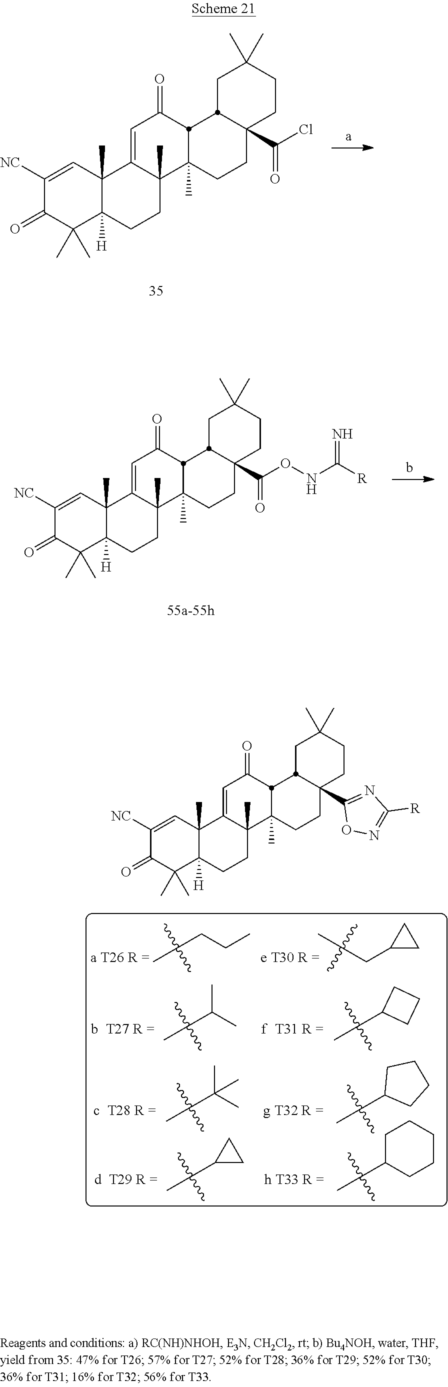

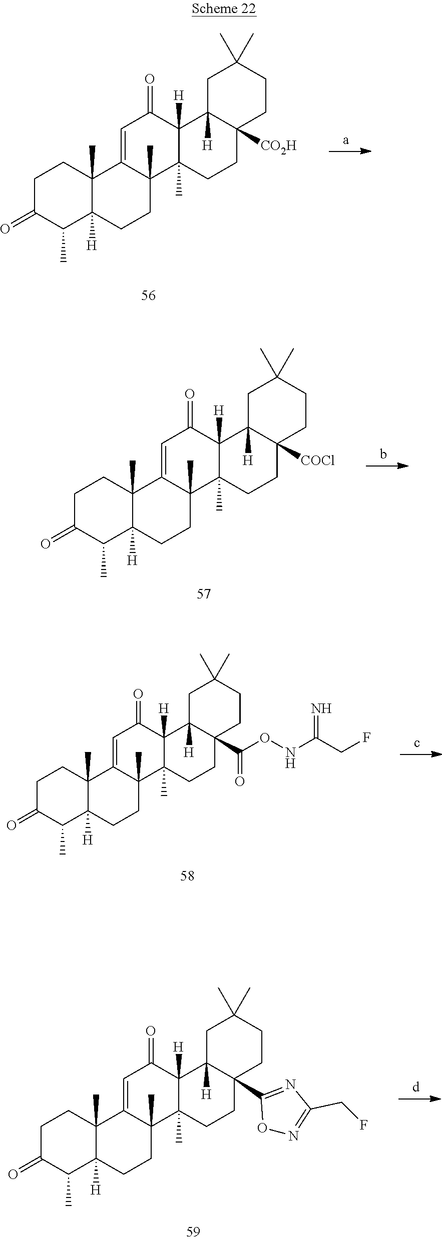

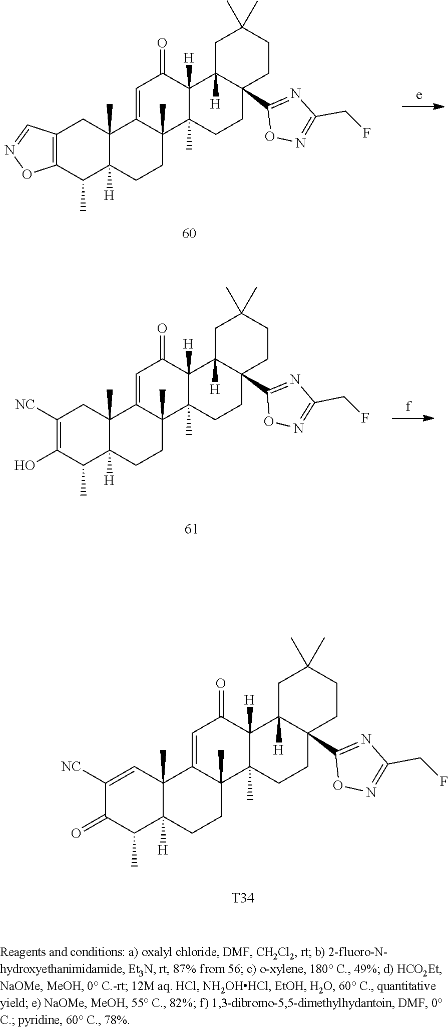

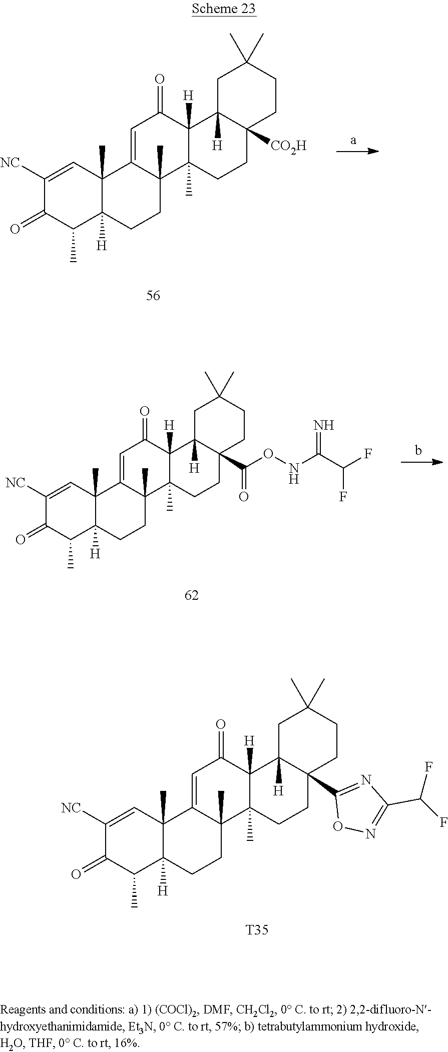

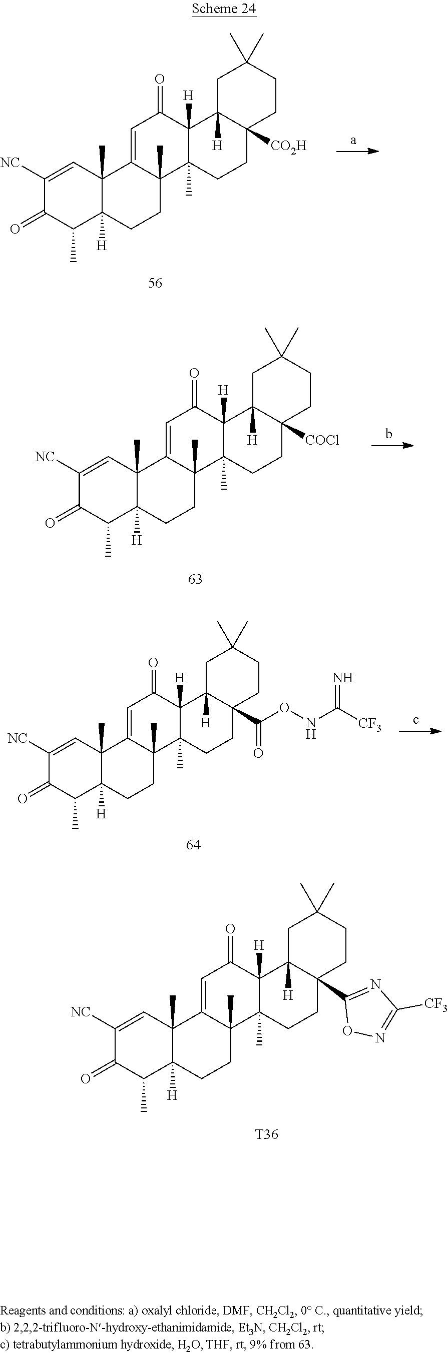

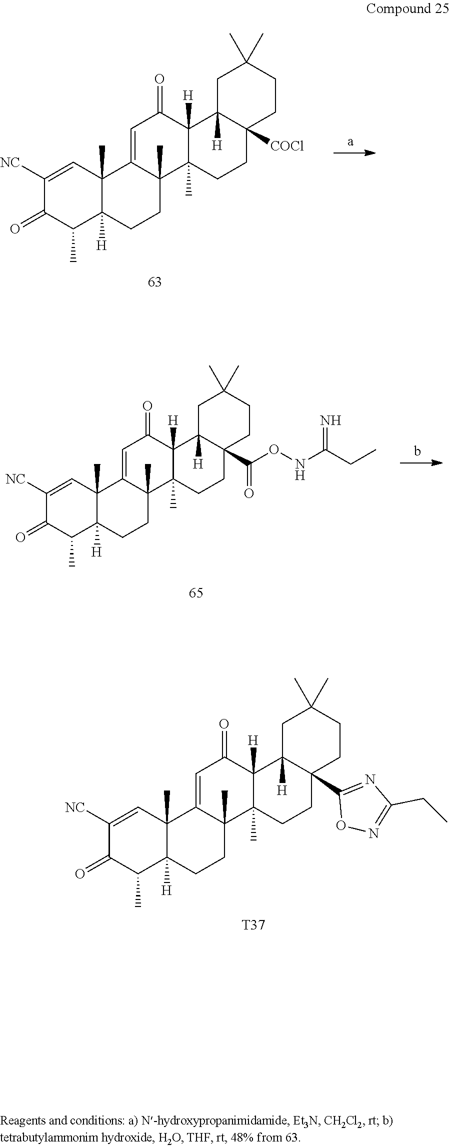

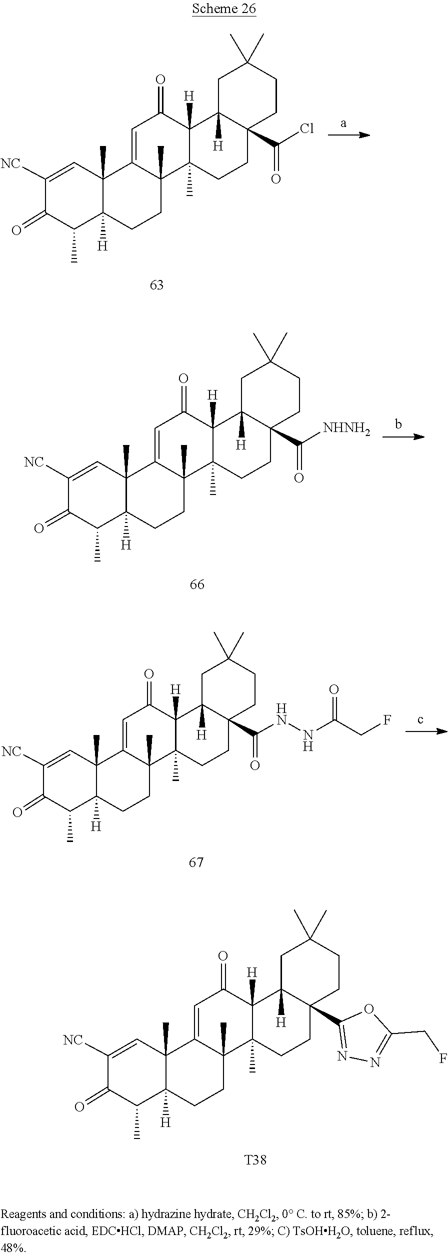

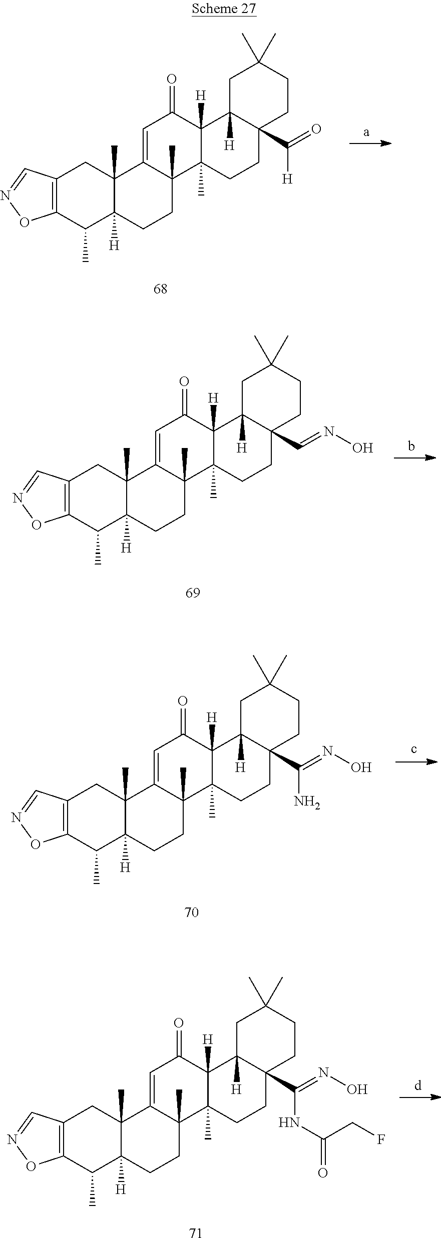

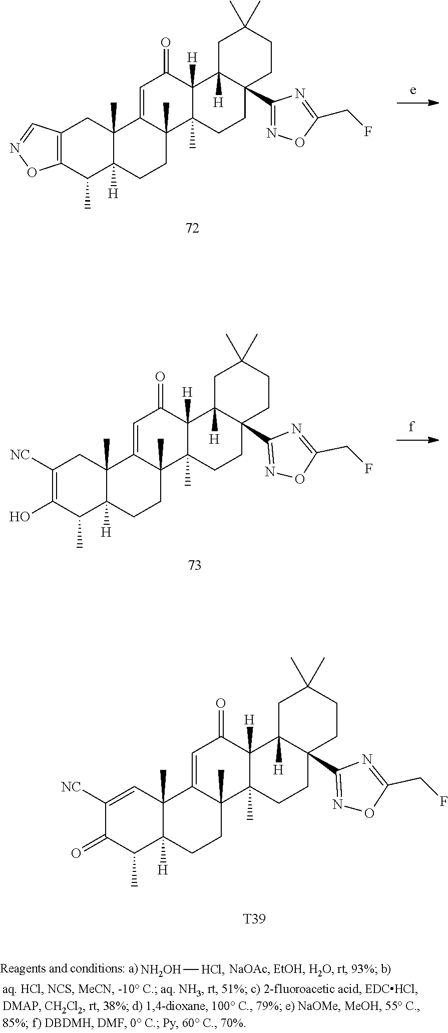

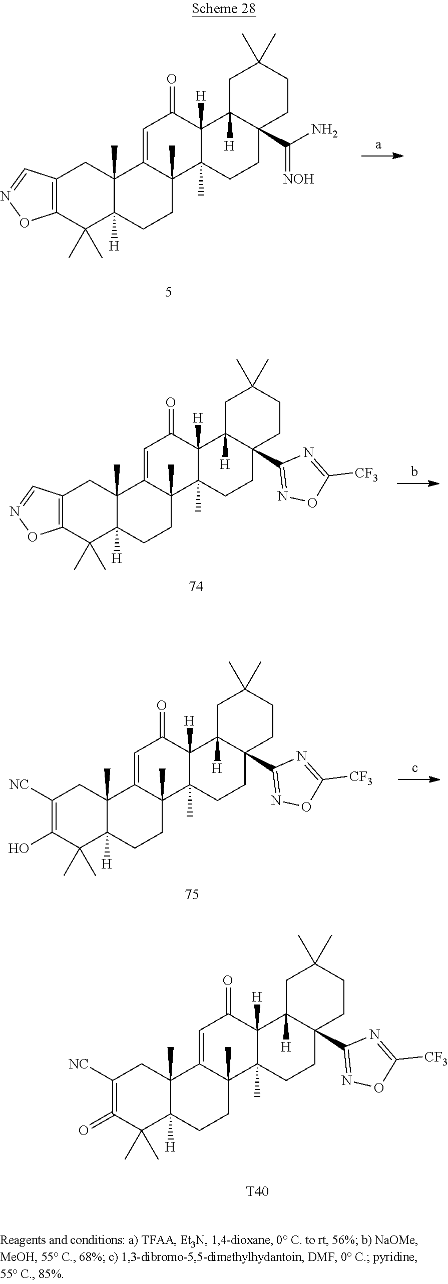

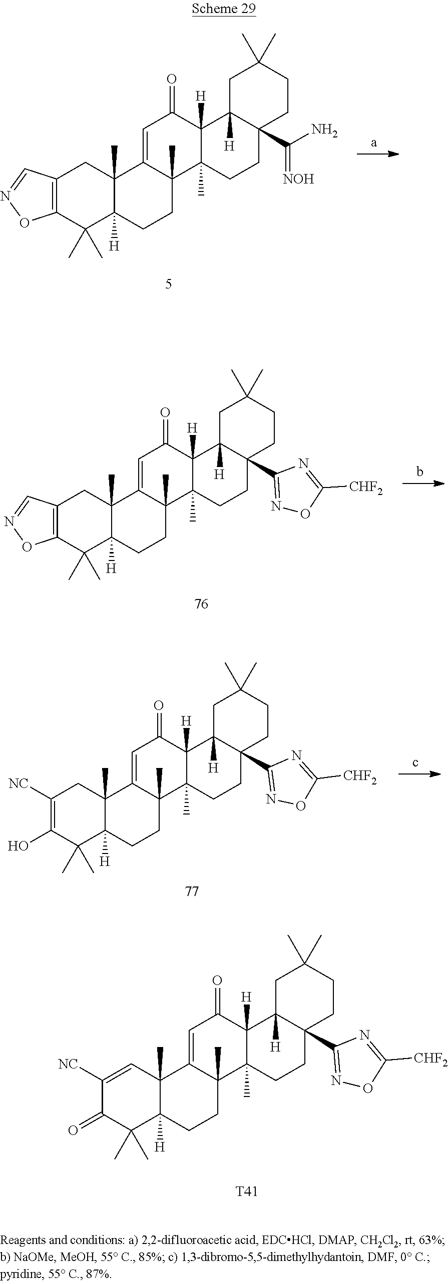

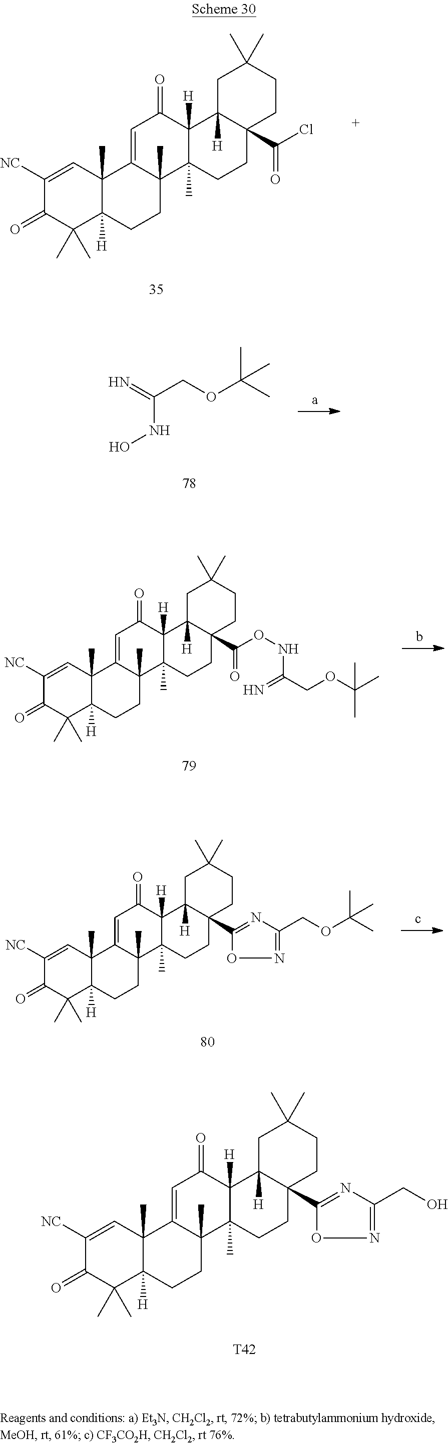

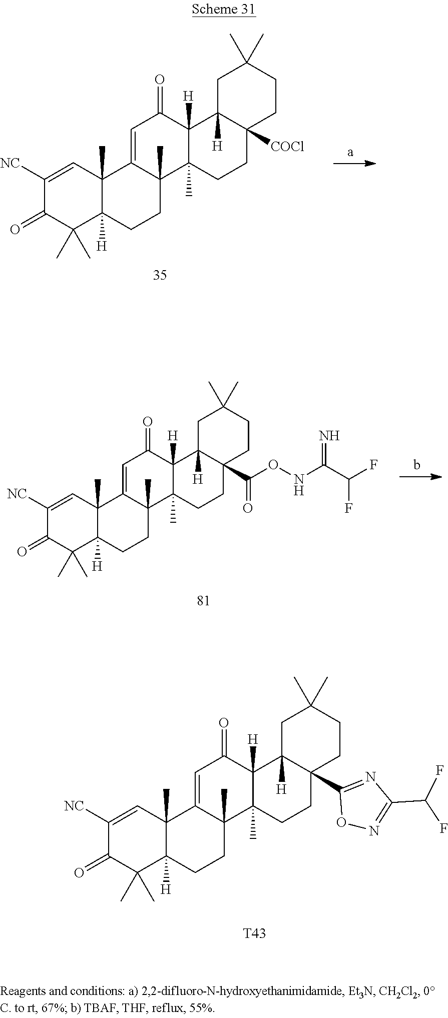

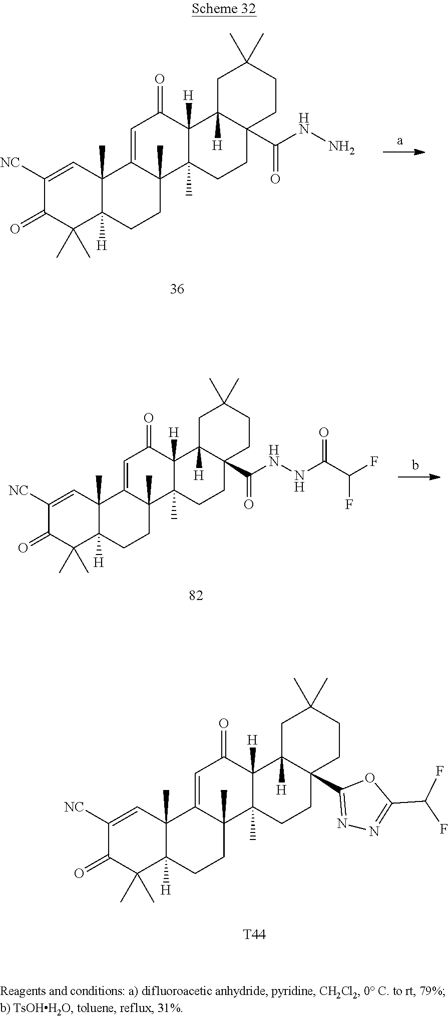

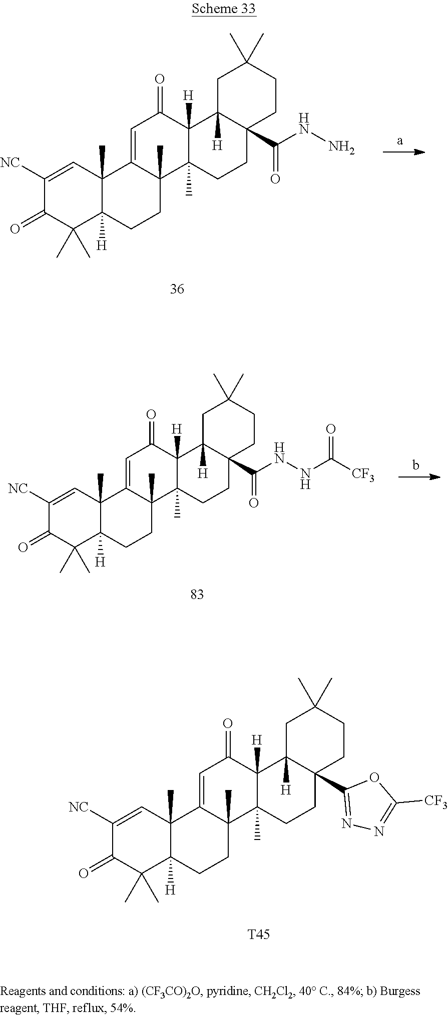

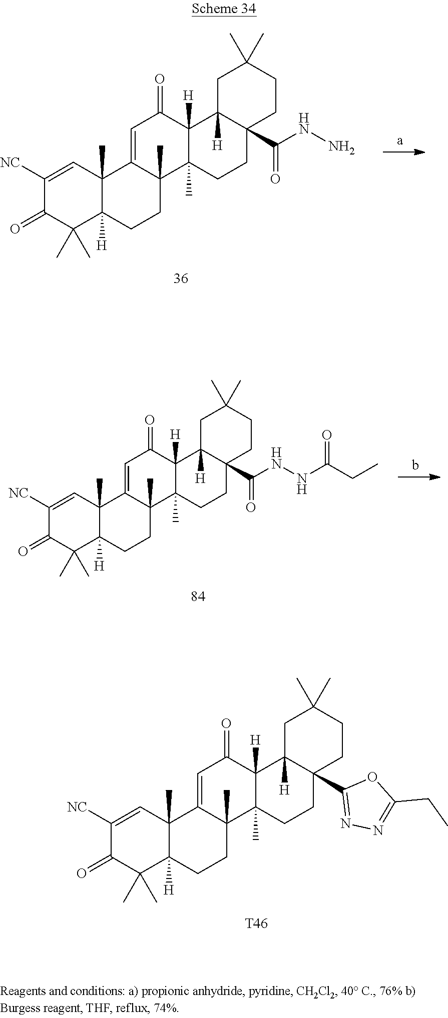

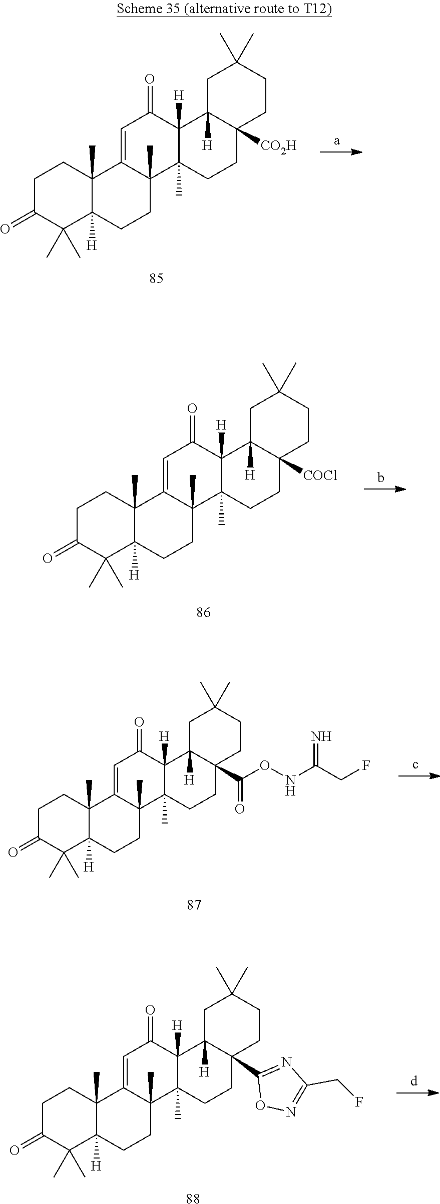

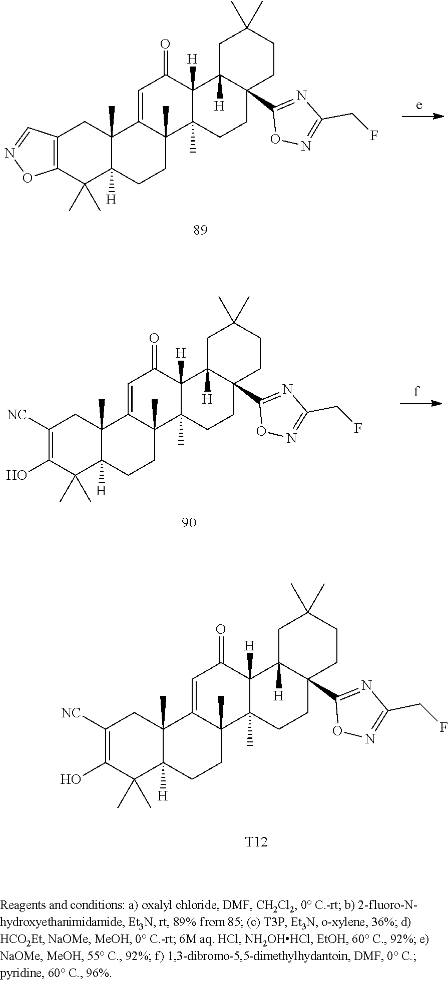

### C. Characterization Data

Compound 2: Compound 1 (10.00 g, 19.71 mmol) in THF (200 mL) was cooled to 0° C. under N₂. DIBAL-H (1.0 M in toluene, 100 mL, 100 mmol) was added. The mixture was stirred at 0° C. for 30 min, and then at room temperature for 2 h. The reaction was cooled to 0° C., and quenched with water (20 mL) carefully, followed by 1 N aq. HCl (300 mL). The mixture was extracted with EtOAc (4×150 mL). The combined organic extracts were washed with water (100 mL) and brine (100 mL); dried with Na₂SO₄; filtered and concentrated to give crude compound 2 (9.5 g, quantitative yield) as a white solid. m/z=482 (M+1).

Compound 3: Compound 2 (9.5 g, ≤19.71 mmol) was dissolved in CH₂Cl₂ (200 mL). 4 Å MS (20 g) and 4-methylmorpholine N-oxide (5.10 g, 43.53 mmol) were added. The mixture was stirred at room temperature for 10 min under N₂. TPAP (690 mg, 1.96 mmol) was added. The mixture was stirred at room temperature for 1.5 h, and then quenched with 10% Na₂SO₃ (50 mL). The mixture was stirred for 5 min at room temperature, and then filtered through a pad of celite. The celite was eluted with CH₂Cl₂ (50 mL). The organic phase from the filtrate was separated. The aqueous phase from the filtrate was extracted with CH₂Cl₂ (2×50 mL), and EtOAc (2×50 mL). The combined organic extracts were washed with water (100 mL); dried with Na₂SO₄; and filtered through a pad of silica gel, which was eluted with EtOAc (100 mL). The filtrate was concentrated. The residue was purified by column chromatography (silica gel, eluting with 0 to 35% EtOAc in hexanes) to give compound 3 (6.39 g, 68% yield) as a white solid. m/z=478 (M+1).

Compound 4: Compound 3 (2.72 g, 5.69 mmol), NH₂OH—HCl (514 mg, 7.40 mmol) and NaOAc (841 mg, 10.2 mmol) were weighed in a flask. EtOH (120 mL) and water (8 mL) were added. The mixture was stirred at room temperature for 14 h, and then concentrated. The residue was partitioned between EtOAc (50 mL) and water (30 mL). The aqueous phase was extracted with EtOAc (30 mL). The combined organic extracts were dried with Na₂SO₄, filtered and concentrated. The residue contains small amount of AcOH and water. EtOH (10 mL) and toluene (10 mL) were added, and then the mixture was concentrated. The residue was triturated with CH₂Cl₂ (30 mL) at reflux for 10 min. After cooled to room temperature, the mixture was kept at room temperature for 30 min. The precipitated solid was collected by filtration; washed with CH₂Cl₂ (2×5 mL); and dried under vacuum to give compound 4 (2.15 g, 77% yield) as a white solid. m/z=493 (M+1).

Compound 5: Under N₂, compound 4 (2.68 g, 5.44 mmol) was suspended in MeCN (11 mL) and cooled to −10° C. Aqueous HCl (12 N, 91 μL, 1.09 mmol) was added, followed by N-chlorosuccinimide (726 mg, 5.44 mmol) in MeCN (11 mL). The reaction was stirred at −10° C. for 30 min. LCMS indicated the starting material was almost all consumed. Aqueous ammonia (28%, 3.7 mL, 54.4 mmol) was added. The mixture was stirred at room temperature for 24 h. The reaction mixture was diluted with EtOAc (100 mL) and washed with water (2×40 mL). The combined aqueous wash was extracted with EtOAc (30 mL). The combined organic extracts were dried with Na₂SO₄, filtered and concentrated. The residue was purified by column chromatography (silica gel, eluting with 0 to 60% EtOAc in CH₂Cl₂) to give compound 5 (1.87 g, 68% yield) as a white solid. m/z=508 (M+1).

Compound 6: A solution of 2-fluoroacetic acid (23 mg, 0.30 mmol) in CH₂Cl₂ (1 mL) was added to N-(3-dimethylaminopropyl)-N′-ethylcarbodiimide hydrochloride (EDC-HCl, 57 mg, 0.30 mmol) at room temperature under N₂. Catalytic amount of DMAP (1.8 mg, 0.015 mmol) was added. The mixture was stirred at room temperature for 15 min. Compound 5 (50 mg, 0.098 mmol) in CH₂Cl₂ (2 mL) was added. The mixture was stirred at room temperature for 1 h. The mixture was diluted with CH₂Cl₂ (20 mL) and washed with water (2×10 mL). The combined aqueous wash was extracted with CH₂Cl₂ (20 mL). The combined organic extracts were dried with Na₂SO₄, filtered and concentrated. The residue was combined with the crude product obtained from compound 5 (11 mg, 0.022 mmol) using the same procedure, and purified by column chromatography (silica gel, eluting with 0 to 50% EtOAc in CH₂Cl₂) to give compound 6 (47 mg, 69% yield) as a white solid. m/z=568 (M+1).

Compound 7: Compound 6 (47 mg, 0.083 mmol) in AcOH (1 mL) was heated at 100° C. for 40 min under N₂. The mixture was cooled to room temperature; diluted with toluene (15 mL); and concentrated. The residue was diluted with toluene (15 mL) again, and concentrated. The residue was purified by column chromatography (silica gel, eluting with 0 to 30% EtOAc in hexanes) to give compound 7 (38 mg, 84% yield) as a white solid. m/z=550 (M+1).

Compound 8: To a mixture of compound 7 (52 mg, 0.095 mmol) in anhydrous MeOH (1 mL) was added NaOMe (4.37 M in MeOH, 43 μL, 0.19 mmol) under N₂. The mixture was heated at 55° C. for 1 h, and then cooled to 0° C. The mixture was diluted with 10% aq. NaH₂PO₄ (15 mL) and extracted with EtOAc (2×15 mL). The combined organic extracts were dried with Na₂SO₄, filtered and concentrated. The residue was purified by column chromatography (silica gel, eluting with 0 to 40% EtOAc in hexanes) to give compound 8 (46 mg, 88% yield) as a white solid. m/z=550 (M+1).

T1: Under N₂, compound 8 (46 mg, 0.084 mmol) was dissolved in anhydrous DMF (0.4 mL), and cooled to 0° C. 1,3-dibromo-5,5-dimethylhydantoin (DBDMH, 13 mg, 0.046 mmol) was added. The mixture was stirred at 0° C. for 1 h. Pyridine (30 μL, 0.38 mmol) was added. The mixture was heated at 55° C. for 6 h, and then cooled to room temperature. The mixture was diluted with EtOAc (25 mL); and washed with 1 N aqueous HCl (10 mL) and water (2×15 mL) sequentially. The organic extract was dried with Na₂SO₄, filtered and concentrated. The residue was purified by column chromatography (silica gel, eluting with 0 to 35% EtOAc in hexanes) to give compound T1 (36 mg, 79% yield) as a white solid. m/z=548 (M+1); ¹H NMR (400 MHz, CDCl₃) δ 8.02 (s, 1H), 5.95 (s, 1H), 5.54 (d, J=46.3 Hz, 2H), 3.21 (m, 1H), 2.96 (d, J=4.7 Hz, 1H), 2.19 (td, J=13.8, 4.2 Hz, 1H), 1.56-1.94 (m, 9H), 1.44 (s, 3H), 1.34-1.52 (m, 3H), 1.25 (s, 3H), 1.16-1.32 (m, 2H), 1.15 (s, 3H), 1.14 (s, 3H), 1.05 (s, 6H), 0.94 (s, 3H).

Compound 9: A mixture of Boc-glycine (207 mg, 1.18 mmol) and N-(3-dimethylaminopropyl)-N′-ethylcarbodiimide hydrochloride (227 mg, 1.18 mmol) in CH₂Cl₂ (4 mL) was treated with DMAP (4.8 mg, 0.039 mmol). The mixture was stirred at room temperature under N₂ for 15 min. Compound 5 (200 mg, 0.39 mmol) in CH₂Cl₂ (4 mL) was added. The mixture was stirred at room temperature for 1 h. Water (15 mL) was added. The mixture was extracted with CH₂Cl₂ (3×15 mL). The combined organic extracts were dried with Na₂SO₄, filtered and concentrated. The residue was purified by column chromatography (silica gel, eluting with 0 to 40% EtOAc in CH₂Cl₂) to give compound 9 (206 mg, 79% yield) as a white solid. m/z=665 (M+1).

Compound 10: A mixture of compound 9 (206 mg, 0.31 mmol) in 1,4-dioxane (4 mL) was heated at 160° C. in a pressure vessel for 100 min. After cooled to room temperature, the reaction mixture was concentration on a rotvap. The residue was dissolved in toluene (10 mL) and concentrated again. The residue was purified by column chromatography (silica gel, eluting with 0 to 60% EtOAc in hexanes) to give compound 10 (160 mg, 80% yield) as a white solid. m/z=647 (M+1).

Compound 11: Compound 10 (159 mg, 0.25 mmol) in MeOH (2.5 mL) was treated with K₂CO₃ (136 mg, 0.98 mmol) at room temperature under N₂. The mixture was stirred at room temperature for 14 h. LCMS indicated the reaction was complete. 10% aq. NaH₂PO₄ (15 mL) was added. The mixture was extracted with EtOAc (2×20 mL). The combined organic extract was dried with Na₂SO₄, filtered and concentrated. The residue was purified by column chromatography (silica gel, eluting with 0 to 70% EtOAc in hexanes) to give compound 11 (141 mg, 89% yield) as a white solid. m/z=669 (M+Na).

Compound 12: Under N₂, compound 11 (140 mg, 0.22 mmol) was dissolved in anhydrous DMF (1.1 mL), and cooled to 0° C. 1,3-dibromo-5,5-dimethylhydantoin (34 mg, 0.12 mmol) was added. The mixture was stirred at 0° C. for 1 h. Pyridine (70 μL, 0.87 mmol) was added. The mixture was heated at 60° C. for 5 h, and then cooled to room temperature. The mixture was diluted with EtOAc (25 mL); and washed with 1 N aq. HCl (10 mL) and water (3×15 mL) sequentially. The organic extract was dried with Na₂SO₄, filtered and concentrated. The residue was purified by column chromatography (silica gel, eluting with 0 to 60% EtOAc in hexanes) to give compound 12 (126 mg, 90% yield) as a white solid. m/z=589 (M-C₄H₇).

T2: Compound 12 (113 mg, 0.18 mmol) in CH₂Cl₂ (1.8 mL) was treated with TFA (135 μL, 1.75 mmol) at room temperature under N₂. After stirring for 5 h at room temperature, sat. aq. NaHCO₃ (15 mL) was added. The mixture was extracted with CH₂Cl₂ (3×15 mL). The combined organic extracts were dried with Na₂SO₄, filtered and concentrated. The residue was purified by column chromatography (silica gel, eluting with 0 to 20% MeOH in CH₂Cl₂) to give compound T2 (47 mg, 49% yield) as a yellow foam. m/z=545 (M+1); ¹H NMR (400 MHz, CDCl₃) δ 8.02 (s, 1H), 5.95 (s, 1H), 4.08 (s, 2H), 3.21 (m, 1H), 2.99 (d, J=4.7 Hz, 1H), 2.17 (td, J=13.6, 4.1 Hz, 1H), 1.44 (s, 3H), 1.25 (s, 3H), 1.15 (s, 6H), 1.12-1.96 (m, 16H), 1.05 (s, 6H), 0.94 (s, 3H).

Compound 13: To a mixture of Boc-P-Ala-OH (168 mg, 0.89 mmol) and EDC-HCl (170 mg, 0.89 mmol) in CH₂Cl₂ (3 mL) was added DMAP (5 mg, 0.04 mmol) at room temperature under N₂. The mixture was stirred at room temperature for 15 min. The solution of compound 5 (150 mg, 0.30 mmol) in CH₂Cl₂ (3 mL) was added. The mixture was stirred at room temperature for 1 h and was then treated with water (15 mL). The mixture was extracted with CH₂Cl₂ (3×15 mL). The combined organic extracts were dried with Na₂SO₄, filtered and concentrated. The residue was purified by column chromatography (silica gel, eluting with 0 to 70% EtOAc in hexanes) to give compound 13 (201 mg, quantitative yield) as a white solid. m/z=679 (M+1).

Compound 14: Compound 13 (200 mg, 0.30 mmol) in 1,4-dioxane (5 mL) was heated at 160° C. for 1 h. The mixture was cooled to room temperature, and concentrated. The residue was purified by column chromatography (silica gel, eluting with 0 to 55% EtOAc in hexanes) to give compound 14 (137 mg, 70% yield) as a white solid. m/z=661 (M+1).

Compound 15: A mixture of compound 14 (135 mg, 0.20 mmol) in MeOH (2 mL) was treated with K₂CO₃ (113 mg, 0.82 mmol) at room temperature. The mixture was stirred at room temperature for 14 h. 10% aq. NaH₂PO₄ (15 mL) was added. The mixture was extracted with EtOAc (30 mL). The organic extract was washed with water (10 mL); dried with Na₂SO₄, filtered and concentrated. The residue was purified by column chromatography (silica gel, eluting with 0 to 70% EtOAc in hexanes) to give compound 15 (114 mg, 84% yield) as a white solid. m/z=683 (M+Na).

Compound 16: A solution of compound 15 (114 mg, 0.17 mmol) and 1,3-dibromo-5,5-dimethylhydantoin (27 mg, 0.095 mmol) in anhydrous DMF (1.7 mL) was stirred at 0° C. for 1 h under N₂. The mixture was treated with pyridine (56 μL, 0.69 mmol), and then heated at 60° C. for 5 h. After cooled to room temperature, the mixture was diluted with EtOAc (25 mL); and washed with 1 N aq. HCl (10 mL) and water (3×15 mL). The organic extract was dried with Na₂SO₄, filtered and concentrated. The residue was purified by column chromatography (silica gel, eluting with 0 to 60% EtOAc in hexanes) to give compound 16 (93 mg, 82% yield) as a white solid. m/z=559 (M-C₅H₇O₂).

T3: Compound 16 (85 mg, 0.13 mmol) in CH₂Cl₂ (0.65 mL) was treated with TFA (99 μL, 1.32 mmol) at room temperature. The mixture was stirred at room temperature for 5 h, and then treated with sat. aq. NaHCO₃ (15 mL). The mixture was extracted with CH₂Cl₂ (3×15 mL). The combined organic extracts were dried with Na₂SO₄, filtered and concentrated. The residue was purified by column chromatography (silica gel, eluting with 0 to 20% MeOH in CH₂Cl₂) to give compound T3 (56 mg, 78% yield) as a yellow solid. m/z=559 (M+1); ¹H NMR (400 MHz, CDCl₃) δ 8.02 (s, 1H), 5.94 (s, 1H), 3.21 (m, 1H), 3.16 (t, J=6.4 Hz, 2H), 3.01 (m, 3H), 2.16 (td, J=13.7, 4.2 Hz, 1H), 1.44 (s, 3H), 1.24 (s, 3H), 1.15 (s, 3H), 1.17-1.94 (m, 16H), 1.14 (s, 3H), 1.05 (s, 3H), 1.04 (s, 3H), 0.94 (s, 3H).

Compound 17: A solution of compound 5 (100 mg, 0.20 mmol) in CH₂Cl₂ (1 mL) was cooled to 0° C. Et₃N (55 μL, 0.39 mmol) in CH₂Cl₂ (0.5 mL), and acetoxyacetyl chloride (40 mg, 0.30 mmol) in CH₂Cl₂ (0.5 mL) were added sequentially. The mixture was stirred at 0° C. for 1 h, and then treated with sat. aq. NaHCO₃ (5 mL). The mixture was stirred for 5 min, and then extracted with CH₂Cl₂ (3×10 mL). The combined organic extracts were dried with Na₂SO₄, filtered and concentrated. The residue was purified by column chromatography (silica gel, eluting with 0 to 60% EtOAc in hexanes) to give compound 17 (119 mg, quantitative yield) as a white solid. m/z=608 (M+1).

Compound 18: Compound 17 (119 mg, 0.20 mmol) in AcOH (1 mL) was heated at 100° C. for 1 h, and then cooled to room temperature. The mixture was diluted with toluene (15 mL), and then concentrated. The residue was diluted with toluene (10 mL) and concentrated again. The residue was purified by column chromatography (silica gel, eluting with 0 to 50% EtOAc in hexanes) to give compound 18 (100 mg, 86% yield) as a white solid. m/z=590 (M+1).

Compound 19: Compound 18 (99 mg, 0.17 mmol) in MeOH (1.7 mL) was treated with NaOMe (4.37 M in MeOH, 0.12 mL, 0.50 mmol) at room temperature. The mixture was heated at 55° C. for 1 h, and then cooled to room temperature. 10% aq. NaH₂PO₄ (10 mL) was added. The mixture was extracted with EtOAc (2×20 mL). The combined organic extracts were dried with Na₂SO₄, filtered and concentrated. The residue was purified by column chromatography (silica gel, eluting with 0 to 70% EtOAc in hexanes) to give compound 19 (83 mg, 90% yield) as a white solid. m/z=548 (M+1).

T4: A solution of compound 19 (82 mg, 0.15 mmol) and 1,3-dibromo-5,5-dimethylhydantoin (23.5 mg, 0.082 mmol) in anhydrous DMF (1.5 mL) was cooled to 0° C. under N₂. The mixture was stirred at 0° C. for 2 h, and then treated with pyridine (48 μL, 0.60 mmol). The mixture was heated at 60° C. for 6 h, and then cooled to room temperature. The mixture was diluted with EtOAc (25 mL) and washed with 1 N aq. HCl (10 mL) and water (2×15 mL). The organic extract was dried with Na₂SO₄, filtered and concentrated. The residue was purified by column chromatography (silica gel, eluting with 0 to 70% EtOAc in hexanes) to give compound T4 (67 mg, 82% yield) as a white solid. m/z=546 (M+1); ¹H NMR (400 MHz, CDCl₃) δ 8.02 (s, 1H), 5.95 (s, 1H), 4.88 (d, J=6.5 Hz, 2H), 3.21 (dt, J=13.7, 4.2 Hz, 1H), 2.97 (d, J=4.7 Hz, 1H), 2.38 (m, 1H), 2.18 (td, J=13.7, 4.2 Hz, 1H), 1.44 (s, 3H), 1.25 (s, 3H), 1.15 (s, 3H), 1.15 (s, 3H), 1.12-1.95 (m, 14H), 1.05 (s, 6H), 0.94 (s, 3H).

Compound 20: 3-Acetoxypropanoic acid (78 mg, 0.59 mmol) in CH₂Cl₂ (2 mL) was added to EDC·HCl (113 mg, 0.59 mmol) at room temperature. DMAP (8 mg, 0.06 mmol) was added. The mixture was stirred at room temperature for 15 min. Compound 5 (100 mg, 0.20 mmol) in CH₂Cl₂ (2 mL) was added. The mixture was stirred at room temperature for 1 h. Sat. aq. NaHCO₃ (3 mL) and water (10 mL) were added. The mixture was diluted with CH₂Cl₂ (20 mL) and EtOAc (2×20 mL). The combined organic extracts were dried with Na₂SO₄, filtered and concentrated. The residue was purified by column chromatography (silica gel, eluting with 0 to 70% EtOAc in hexanes) to give compound 20 (80 mg, 66% yield) as a white solid. m/z=622 (M+1).

Compound 21: Compound 20 (77 mg, 0.12 mmol) was dissolved in AcOH (1 mL) and heated at 100° C. for 2 h. After cooled to room temperature, the mixture was diluted with toluene (10 mL), and concentrated. The residue was diluted with toluene (10 mL) and concentrated again. The residue was purified by column chromatography (silica gel, eluting with 0 to 50% EtOAc in hexanes) to give compound 21 (41 mg, 55% yield) as a white solid. m/z=604 (M+1).

Compound 22 and 23: Compound 21 (40 mg, 0.066 mmol) in MeOH (1.2 mL) was treated with NaOMe (4.37 M in MeOH, 45 μL, 0.20 mmol) at room temperature. The mixture was heated at 55° C. for 1 h, and then cooled to room temperature. 10% aq. NaH₂PO₄ (10 mL) was added. The mixture was extracted with EtOAc (2×20 mL). The combined organic extracts were dried with Na₂SO₄, filtered and concentrated. The residue was purified by column chromatography (silica gel, eluting with 0 to 70% EtOAc in hexanes) to give compound 22 (19 mg, 51% yield) and compound 23 (17 mg, 45% yield) as white solids. Compound 22: m/z=562 (M+1); Compound 23: m/z=576 (M+1).

T5: A solution of compound 22 (19 mg, 0.034 mmol) in anhydrous DMF (0.3 mL) was cooled to 0° C. 1,3-dibromo-5,5-dimethylhydantoin (5.3 mg, 0.019 mmol) in DMF (55 L) was added. The mixture was stirred at 0° C. for 1 h, and then treated with pyridine (11 μL, 0.14 mmol). The mixture was heated at 60° C. for 5 h, and then cooled to room temperature. The mixture was diluted with EtOAc (25 mL) and washed with 1 N aq. HCl (10 mL) and water (2×15 mL) sequentially. The organic extract was dried with Na₂SO₄, filtered and concentrated. The residue was purified by column chromatography (silica gel, eluting with 0 to 70% EtOAc in hexanes) to give compound T5 (16 mg, 86% yield) as a white solid. m/z=560 (M+1); ¹H NMR (400 MHz, CDCl₃) δ 8.02 (s, 1H), 5.95 (s, 1H), 4.05 (q, J=5.8 Hz, 2H), 3.19 (m, 1H), 3.11 (t, J=5.6 Hz, 2H), 3.01 (d, J=4.7 Hz, 1H), 2.58 (t, J=6.5 Hz, 1H), 2.17 (td, J=13.7, 4.1 Hz, 1H), 1.44 (s, 3H), 1.25 (s, 3H), 1.15 (s, 3H), 1.14 (s, 3H), 1.13-1.95 (m, 14H), 1.04 (s, 6H), 0.94 (s, 3H).

T6: A solution of compound 23 (17 mg, 0.030 mmol) in anhydrous DMF (0.3 mL) was cooled to 0° C. 1,3-dibromo-5,5-dimethylhydantoin (4.6 mg, 0.016 mmol) in DMF (46 L) was added. The mixture was stirred at 0° C. for 1 h, and then treated with pyridine (9.5 μL, 0.12 mmol). The mixture was heated at 60° C. for 5 h, and then cooled to room temperature. The mixture was diluted with EtOAc (25 mL) and washed with 1 N aq. HCl (10 mL) and water (2×15 mL) sequentially. The organic extract was dried with Na₂SO₄, filtered and concentrated. The residue was purified by column chromatography (silica gel, eluting with 0 to 70% EtOAc in hexanes) to give compound T6 (14 mg, 83% yield) as a white solid. m/z=574 (M+1); ¹H NMR (400 MHz, CDCl₃) δ 8.02 (s, 1H), 5.94 (s, 1H), 3.79 (t, J=6.5 Hz, 2H), 3.34 (s, 3H), 3.22 (m, 1H), 3.13 (t, J=6.5 Hz, 2H), 3.00 (d, J=4.7 Hz, 1H), 2.16 (td, J=13.6, 4.2 Hz, 1H), 1.44 (s, 3H), 1.24 (s, 3H), 1.15 (s, 3H), 1.14-1.94 (m, 14H), 1.13 (s, 3H), 1.05 (s, 3H), 1.04 (s, 3H), 0.94 (s, 3H).

Compound 25: To a mixture of compound 24 (1.366 g, 3.04 mmol) in o-xylene (5 mL) was added azidotributyltin(IV) (1.00 mL, 3.65 mmol). The mixture was heated at 150° C. for 48 h. The crude reaction mixture was purified by column chromatography (silica gel, eluting with 0 to 60% acetone in hexanes) to give partially purified compound 25, which was purified again by column chromatography (silica gel, eluting with 0 to 100% EtOAc in hexanes) to give compound 25 (710 mg, 47% yield) as a brown solid. m/z=493 (M+1).

Compound 26: A mixture of compound 25 (200 mg, 0.41 mmol) and Cs₂CO₃ (160 mg, 0.49 mmol) in MeCN (4 mL) was treated with 2-bromoethanol (40 μL, 0.56 mmol) at room temperature. The mixture was heated at 60° C. for 3 h, and then additional amount of 2-bromoethanol (40 μL, 0.56 mmol) was added. The mixture was heated at 60° C. for another 3 h and cooled to room temperature.

The mixture was diluted with EtOAc (50 mL) and filtered. The filtrate was concentrated. The residue was combined with the crude product obtained from compound 25 (50 mg, 0.10 mmol), and was purified by column chromatography (silica gel, eluting with 0 to 50% acetone in hexanes) to give compound 26 (222 mg, 82% yield) as a white solid. m/z=537 (M+1).

Compound 27: A mixture compound 26 (220 mg, 0.41 mmol) in ethyl formate (1.00 mL, 12.29 mmol) was cooled to 0° C. under N₂ and treated dropwise with sodium methoxide solution (4.37 M in methanol, 1.40 mL, 6.12 mmol). The reaction mixture was stirred at room temperature for 1.5 h, and then cooled to 0° C. The mixture was treated with 6 N aq. HCl (1.1 mL, 6.6 mmol), followed by EtOH (8 mL) and hydroxylamine hydrochloride (43 mg, 0.62 mmol). The reaction mixture was heated at 55° C. for 5 h; cooled to room temperature; and concentrated. The residue was diluted with EtOAc and washed with water. The organic extract was dried with Na₂SO₄; filtered; and concentrated. The residue was purified by column chromatography (silica gel, eluting with 0 to 100% EtOAc in hexanes) to give compound 27 (166 mg, 72% yield) as a white solid. m/z=562 (M+1).

Compound 28: To a mixture of compound 27 (166 mg, 0.30 mmol) in anhydrous MeOH (3 mL) was added NaOMe (4.37 M in MeOH, 0.14 mL, 0.61 mmol) under N₂. The mixture was heated at 55° C. for 1 h, and then cooled to room temperature. The mixture was diluted with 10% aq. NaH₂PO₄ and extracted twice with EtOAc. The combined organic extracts were dried with Na₂SO₄, filtered and concentrated to give compound 28 (180 mg, quantitative yield) as a white solid. m/z=562 (M+1).

T7: Under N₂, compound 28 (180 mg, 0.30 mmol) was dissolved in anhydrous DMF (1 mL), and cooled to 0° C. 1,3-dibromo-5,5-dimethylhydantoin (42 mg, 0.15 mmol) was added. The mixture was stirred at 0° C. for 1 h. Pyridine (72 μL, 0.89 mmol) was added. The mixture was heated at 55° C. for 16 h, and then cooled to room temperature. The mixture was diluted with EtOAc and washed with 1 N aqueous HCl and water (3×) sequentially. The organic extract was dried with Na₂SO₄, filtered and concentrated. The residue was purified by column chromatography (silica gel, eluting with 0 to 70% EtOAc in hexanes) to give compound T7 (114 mg, 68% yield) as a white solid. m/z=560 (M+1); ¹H NMR (400 MHz, CDCl₃) δ 8.01 (s, 1H), 5.94 (s, 1H), 4.72 (dd, J=5.9, 4.2 Hz, 2H), 4.16 (dd, J=6.1, 4.5 Hz, 2H), 3.32 (dt, J=13.7, 4.2 Hz, 1H), 2.97 (d, J=4.7 Hz, 1H), 2.24 (m, 1H), 2.21 (t, J=6.3 Hz, 1H), 2.03 (m, 1H), 1.83 (td, J=13.9, 4.5 Hz, 1H), 1.42 (s, 3H), 1.24 (s, 3H), 1.14 (s, 3H), 1.12-1.78 (m, 12H), 1.06 (s, 3H), 1.05 (s, 3H), 0.99 (s, 3H), 0.95 (s, 3H).

T8: The solution of compound T7 (30 mg, 0.054 mmol) in CH₂Cl₂ (0.5 mL) was cooled to 0° C. Pyridine (13 μL, 0.16 mmol), acetic anhydride (10 μL, 0.11 mmol) and catalytic amount of DMAP were added sequentially. The mixture was stirred at 0° C. for 2.5 h and was diluted with toluene (5 mL). The mixture was concentrated. The residue was purified by column chromatography (silica gel, eluting with 0 to 45% EtOAc in hexanes) to give compound T8 (23 mg, 71% yield) as a white solid. m/z=602 (M+1); ¹H NMR (400 MHz, CDCl₃) δ 8.02 (s, 1H), 5.94 (s, 1H), 4.82 (m, 2H), 4.54 (m, 2H), 3.31 (m, 1H), 2.95 (d, J=4.7 Hz, 1H), 2.23 (td, J=13.7, 4.1 Hz, 1H), 2.03 (m, 1H), 2.00 (s, 3H), 1.81 (td, J=13.9, 4.5 Hz, 1H), 1.42 (s, 3H), 1.24 (s, 3H), 1.14 (s, 3H), 1.12-1.76 (m, 12H), 1.06 (s, 3H), 1.05 (s, 3H), 0.98 (s, 3H), 0.95 (s, 3H).

T9: A mixture of compound T7 (72 mg, 0.13 mmol), Proton sponge (82 mg, 0.38 mmol) and trimethyloxonium tetrafluoroborate (56 mg, 0.38 mmol) in CH₂Cl₂ (1.2 mL) was stirred at room temperature for 16 h. The reaction was quenched with sat. aq. NaHCO₃; stirred for 5 min; and extracted with EtOAc. The organic extract was washed with water, 1 N aq. HCl, and water sequentially; dried with Na₂SO₄; filtered and concentrated. The residue was purified by column chromatography (silica gel, eluting with 0 to 80% EtOAc in hexanes) to give compound T9 (32 mg, 43% yield) as a white solid. m/z=574 (M+1); ¹H NMR (400 MHz, CDCl₃) δ 8.02 (s, 1H), 5.94 (s, 1H), 4.74 (m, 2H), 3.89 (t, J=5.4 Hz, 2H), 3.32 (m, 1H), 3.29 (s, 3H), 2.99 (d, J=4.7 Hz, 1H), 2.22 (td, J=13.6, 4.1 Hz, 1H), 2.05 (m, 1H), 1.82 (td, J=13.9, 4.5 Hz, 1H), 1.42 (s, 3H), 1.23 (s, 3H), 1.14 (s, 3H), 1.12-1.76 (m, 12H), 1.06 (s, 3H), 1.05 (s, 3H), 0.98 (s, 3H), 0.95 (s, 3H).

Compound 29: In a vial, a mixture of compound 25 (200 mg, 0.41 mmol) and Cs₂CO₃ (160 mg, 0.49 mmol) in MeCN (2 mL) was treated with 1-fluoro-2-iodoethane (100 mg, 0.58 mmol) in MeCN (2 mL). The vial was sealed and heated at 60° C. for 6 h. After cooled to room temperature, the mixture was diluted with EtOAc, and filtered through a pad of celite. The filter cake was washed with EtOAc. The combined filtrate and wash were concentrated. The residue was combined with the crude product obtained from compound 25 (50 mg, 0.10 mmol), and was purified by column chromatography (silica gel, eluting with 0 to 50% EtOAc in hexanes) to give compound 29 (142 mg, 52% yield) as a light yellow solid. m/z=539 (M+1).

Compound 30: A mixture compound 29 (142 mg, 0.26 mmol) in ethyl formate (0.64 mL, 7.86 mmol) was cooled to 0° C. under N₂ and treated dropwise with sodium methoxide solution (4.37 M in methanol, 0.90 mL, 3.93 mmol). The reaction mixture was stirred at room temperature for 1.5 h, and then cooled to 0° C. The mixture was treated with 6 N aq. HCl (0.66 mL, 3.96 mmol), followed by EtOH (5.2 mL) and hydroxylamine hydrochloride (28 mg, 0.40 mmol). The reaction mixture was heated at 55° C. for 5 h; cooled to room temperature; and concentrated. The residue was diluted with EtOAc and washed with water. The organic extract was dried with Na₂SO₄; filtered; and concentrated. The residue was purified by column chromatography (silica gel, eluting with 0 to 50% EtOAc in hexanes) to give compound 30 (102 mg, 71% yield) as a white solid. m/z=544 (M+1).

Compound 31: Compound 30 (102 mg, 0.19 mmol) was dissolved in MeOH (1 mL) and CH₂Cl₂ (1 mL), and cooled to −78° C. Ozone was bubbled through the reaction mixture until the starting material was completely consumed (˜10 min). Oxygen was bubbled for 5 min. NaBH₄ (15 mg, 0.40 mmol) was added. The cold bath was removed. The mixture was stirred at room temperature for 3 h; diluted with EtOAc; and washed with 1 N aq. HCl and water. The aqueous washes were combined and extracted with EtOAc. The combined organic extracts were dried with Na₂SO₄; filtered; and concentrated to give compound 31 (96 mg, 99% yield) as a white solid. m/z=518 (M+1).

Compound 32: A mixture of compound 31 (96 mg, 0.19 mmol) and Cs₂CO₃ (73 mg, 0.22 mmol) was treated with 1-fluoro-2-iodoethane (45 mg, 0.26 mmol) in MeCN (1.8 mL). The mixture was heated at 60° C. under N₂ for 6 h. After cooled to room temperature, the mixture was diluted with EtOAc, and washed with 1 N aq. HCl, 10% aq. Na₂SO₃, and water sequentially. The organic extract was dried with Na₂SO₄; filtered; and concentrated. The residue was purified by column chromatography (silica gel, eluting with 0 to 50% EtOAc in hexanes) to give compound 32 (63 mg, 60% yield) as a white solid. m/z=564 (M+1).

Compound 33: A mixture of compound 32 (61 mg, 0.11 mmol) in MeOH (1 mL) was treated with K₂CO₃ (45 mg, 0.33 mmol) at room temperature. The mixture was stirred at room temperature for 16 h. 10% aq. NaH₂PO₄ (15 mL) was added. The mixture was extracted with twice with EtOAc. The combined organic extracts were dried with Na₂SO₄, filtered and concentrated. The residue was purified by column chromatography (silica gel, eluting with 0 to 50% EtOAc in hexanes) to give compound 33 (48 mg, 79% yield) as a white solid. m/z=564 (M+1).

T10: A solution of compound 33 (48 mg, 0.085 mmol) in anhydrous DMF (0.42 mL) was cooled to 0° C. under N₂. 1,3-dibromo-5,5-dimethylhydantoin (12 mg, 0.042 mmol) was added. The mixture was stirred at 0° C. for 1 h, and then treated with pyridine (21 μL, 0.26 mmol). The mixture was heated at 55° C. for 16 h. After cooled to room temperature, the mixture was diluted with EtOAc; and washed with 1 N aq. HCl and water (3×) sequentially. The organic extract was dried with Na₂SO₄, filtered and concentrated. The residue was purified by column chromatography (silica gel, eluting with 0 to 50% EtOAc in hexanes) to give compound T10 (34 mg, 71% yield) as a white solid. m/z=562 (M+1); ¹H NMR (400 MHz, CDCl₃) δ 8.01 (s, 1H), 5.94 (s, 1H), 4.78-5.01 (m, 4H), 3.32 (dt, J=13.6, 4.3 Hz, 1H), 2.99 (d, J=4.7 Hz, 1H), 2.24 (td, J=13.6, 4.1 Hz, 1H), 2.04 (m, 1H), 1.82 (td, J=13.9, 4.5 Hz, 1H), 1.42 (s, 3H), 1.23 (s, 3H), 1.13 (s, 3H), 1.12-1.77 (m, 12H), 1.06 (s, 3H), 1.05 (s, 3H), 0.96 (s, 3H), 0.95 (s, 3H).

Compound 35: Compound 34 (1.00 g, 2.03 mmol) was mixed with CH₂Cl₂ (20 mL) and cooled to 0° C. under N₂. Oxalyl chloride (0.54 mL, 6.17 mmol) and DMF (16 μL, 0.20 mmol) were added sequentially. The cold bath was removed. The mixture was stirred at room temperature for 2 h, and then concentrated. The residue was dissolved in toluene (3×10 mL) and concentrated to remove residual oxalyl chloride. Crude compound 35 (1 g) was obtained as a light yellow solid and used in the next step without further purification.

Compound 36: Compound 35 (200 mg, 0.39 mmol) in CH₂Cl₂ (4 mL) was cooled to 0° C. under N₂. Hydrazine hydrate (50 wt. %, 75 mg, 1.18 mmol) was added dropwise. The mixture was stirred at room temperature for 10 min; diluted with CH₂Cl₂ (10 mL); and washed with water (15 mL). The aqueous wash was extracted with CH₂Cl₂ (2×15 mL) and EtOAc (15 mL). The combined organic extracts were dried with Na₂SO₄, filtered and concentrated to give compound 36 (179 mg, 90% yield) as a glass.

Compound 37: A solution of 2-fluoroacetic acid (67 mg, 0.86 mmol) in CH₂Cl₂ (1 mL) was treated with EDC-HCl (164 mg, 0.85 mmol) and DMAP (3.5 mg, 0.028 mmol) at room temperature under N₂. The mixture was stirred at room temperature for 15 min. Compound 36 (144 mg, 0.29 mmol) in CH₂Cl₂ (2 mL) was then added. The mixture was stirred at room temperature for 14 h. The reaction mixture was washed with sat. aq. NaHCO₃ (10 mL) and water (10 mL). The aqueous phase was extracted with CH₂Cl₂ (2×10 mL). The combined organic extracts were dried with Na₂SO₄, filtered and concentrated. The residue was purified by column chromatography (silica gel, eluting with 0 to 60% EtOAc in CH₂Cl₂) to give compound 37 (96 mg, 60% yield) as a pink solid. m/z=566 (M+1).

T11: Compound 37 (115 mg, 0.20 mmol) and p-toluenesulfonic acid monohydrate (19 mg, 0.10 mmol) in toluene (10 mL) was heated at reflux while removing water by Dean-Stark apparatus for 3 h. The mixture was cooled to room temperature; diluted with EtOAc (10 mL); and washed with water (2×15 mL). The organic extract was dried with Na₂SO₄, filtered and concentrated. The residue was purified by column chromatography (silica gel, eluting with 0 to 40% acetone in hexanes) to give compound T11 (67 mg, 60% yield) as a white solid. m/z=548 (M+1); ¹H NMR (400 MHz, CDCl₃) δ 8.02 (s, 1H), 5.96 (s, 1H), 5.51 (dd, J=47.0 Hz, 2H), 3.18 (dt, J=13.5, 4.3 Hz, 1H), 2.99 (d, J=4.7 Hz, 1H), 2.23 (td, J=13.7, 4.2 Hz, 1H), 1.45 (s, 3H), 1.25 (s, 3H), 1.20-2.02 (m, 14H), 1.15 (s, 6H), 1.06 (s, 3H), 1.05 (s, 3H), 0.96 (s, 3H).

Compound 39: A solution of compound 35 [produced from compound 34 (1.52 g, 3.09 mmol)] in CH₂Cl₂ (25 mL) was cooled to 0° C. Et₃N (1.70 mL, 12.4 mmol) and a solution of compound 38 (404 mg, 4.39 mmol) in CH₂Cl₂ (5 mL) were added sequentially. After stirring at room temperature for 4 h, the mixture was treated with water (30 mL). The organic phase was separated. The aqueous phase was extracted with CH₂Cl₂ (3×30 mL). The combined organic extracts were dried with Na₂SO₄, filtered and concentrated. The residue was purified by column chromatography (silica gel, eluting with 0 to 60% acetone in hexanes) to give compound 39 (1.46 g, 84% yield) as a white solid. m/z=566 (M+1).

T12: A mixture of compound 39 (148 mg, 0.26 mmol), T3P (50 wt. % in EtOAc, 0.40 g, 0.63 mmol) and Et₃N (0.18 mL, 1.31 mmol) in EtOAc (1 mL) were heated at 125° C. in Biotage microwave synthesizer for 1 h. After cooled to room temperature, the mixture was diluted with EtOAc (20 mL) and washed with 1 N aq. HCl (15 mL), sat. aq. NaHCO₃ (15 mL) and water (15 mL) sequentially. The organic extract was dried with Na₂SO₄, filtered, and concentrated. The residue was purified by column chromatography (silica gel, eluting with 0 to 80% EtOAc in hexanes) to give compound T12 (9 mg, 6% yield) as a white solid. m/z=548 (M+1); ¹H NMR (400 MHz, CDCl₃) δ 8.02 (s, 1H), 5.97 (s, 1H), 5.44 (d, J=46.5 Hz, 2H), 3.26 (m, 1H), 3.03 (d, J=4.7 Hz, 1H), 2.24 (td, J=13.8, 4.1 Hz, 1H), 1.87-2.01 (m, 2H), 1.45 (s, 3H), 1.25 (s, 3H), 1.20-1.80 (m, 12H), 1.15 (s, 3H), 1.10 (s, 3H), 1.06 (s, 3H), 1.05 (s, 3H), 0.95 (s, 3H).

A stock solution of compound 35 (≤0.183 M) and trimethylamine (0.735 M) in CH₂Cl₂ was prepared by dissolving compound 35 (prepared from 11.11 mmol of compound 34) and triethylamine (6.20 mL, 44.5 mmol) in CH₂Cl₂ (52 mL). The total volume of the solution was 60.5 mL. The stock solution was used in the synthesis of compound 40 and 41.

Compound 40: The stock solution of compound 35 and Et₃N in CH₂Cl₂ [12.5 mL, containing compound 35 (2.29 mmol) and trimethylamine (9.19 mmol)] was treated with 3-hydroxypropionamide oxime (347 mg, 3.33 mmol). The mixture was stirred overnight at room temperature. The resultant mixture was diluted with EtOAc; washed with water, sat. aq. NaHCO₃ and brine. The organic extract was dried with Na₂SO₄; filtered and concentrated. The residue was purified by column chromatography (silica gel, eluting with 0 to 10% MeOH in CH₂Cl₂) to give compound 40 (604.4 mg, 46% yield from compound 34) as a solid.

T13: A mixture of compound 40 (604.4 mmol, 1.046 mmol) and tetrabutylammonium hydroxide (40% w/w in water, 2.1 mL, 3.2 mmol) in THF (8.4 mL) was stirred at room temperature under N₂ overnight. The resultant mixture was diluted with EtOAc; washed with water, sat. aq. NaHCO₃ and brine. The organic extract was dried with Na₂SO₄; filtered and concentrated. The residue was purified by column chromatography (silica gel, eluting with 0 to 100% EtOAc in hexanes) to give compound T13 (265.5 mg, 45% yield) as a white solid. m/z=560.3 (M+1); ¹H NMR (400 MHz, CDCl₃) δ 8.02 (s, 1H), 5.96 (s, 1H), 3.98 (q, J=6.0 Hz, 2H), 3.24 (td, J=4.2, 13.9 Hz, 1H), 3.03 (d, J=4.7 Hz, 1H), 2.99 (t, J=5.6 Hz, 2H), 2.33 (t, J=6.3 Hz, 1H), 2.21 (dt, J=4.1, 13.8 Hz, 1H), 1.91 (m, 2H), 1.69 (m, 7H), 1.45 (s, 3H), 1.33 (m, 5H), 1.25 (s, 3H), 1.15 (s, 3H), 1.11 (s, 3H), 1.05 (s, 3H), 1.04 (s, 3H), 0.95 (s, 3H).

T14: A mixture of compound T13 (101.2 mg, 0.1808 mmol) and HCl (12 M aq, 0.5 mL, 6.0 mmol) in glacial acetic acid (10 mL) was stirred at 75° C. under N₂ for 20 h. The resultant mixture was azeotroped with toluene (60 mL, then 50 mL), and the residue was purified by column chromatography (silica gel, eluting with 0 to 100% EtOAc in hexanes) to give compound T14 (48.0 mg, 44% yield) as a white solid. m/z=602.3 (M+1); ¹H NMR (400 MHz, CDCl₃) δ 8.02 (s, 1H), 5.96 (s, 1H), 4.41 (m, 2H), 3.23 (td, J=3.9, 13.3 Hz, 1H), 3.05 (m, 3H), 2.20 (dt, J=4.1, 13.8 Hz, 1H), 2.02 (s, 3H), 1.90 (m, 2H), 1.70 (m, 7H), 1.45 (s, 3H), 1.35 (m, 5H), 1.24 (s, 3H), 1.15 (s, 3H), 1.10 (s, 3H), 1.04 (s, 3H), 1.04 (s, 3H), 0.94 (s, 3H).

Compound 41: The stock solution of compound 35 and Et₃N in CH₂Cl₂[12.5 mL, containing compound 35 (2.29 mmol) and trimethylamine (9.19 mmol)] was treated with 3-methoxypropionamidoxime hydrochloride (516 mg, 3.33 mmol) and additional triethylamine (0.46 mL, 3.3 mmol). The mixture stirred overnight at room temperature. The resultant mixture was diluted with EtOAc; washed with water, sat. aq. NaHCO₃ and brine. The organic extract was dried with Na₂SO₄; filtered and concentrated. The residue was purified by column chromatography (silica gel, eluting with 0 to 10% MeOH in CH₂Cl₂) to give a mixture of compound 41 and compound 34 (1.22 g, -2.5:1-41:34) as a solid that was used without further purification.

T15: A mixture of compound 41 contaminated with compound 34 (1.22 g, -2.5:1-41:34) and tetrabutylammonium hydroxide (40% w/w in water, 4.2 mL, 6.4 mmol) in THF (17 mL) was stirred at room temperature under N₂ overnight. The resultant mixture was diluted with EtOAc; washed with water, sat. aq. NaHCO₃ and brine. The organic extract was dried with Na₂SO₄; filtered and concentrated. The residue was purified by column chromatography (silica gel, eluting with 0 to 100% EtOAc in hexanes) to give compound T15 (494.7 mg, 37% yield from compound 34) as a white solid. m/z=574.4 (M+1); ¹H NMR (400 MHz, CDCl₃) δ 8.02 (s, 1H), 5.96 (s, 1H), 3.74 (t, J=6.6 Hz, 2H), 3.33 (s, 3H), 3.24 (td, J=4.1, 13.6 Hz, 1H), 3.09 (d, J=4.7 Hz, 1H), 2.99 (dt, J=2.6, 6.6 Hz, 2H), 2.20 (dt, J=4.1, 13.8 Hz, 1H), 1.90 (m, 2H), 1.71 (m, 7H), 1.45 (s, 3H), 1.35 (s, 5H), 1.25 (s, 3H), 1.15 (s, 3H), 1.11 (s, 3H), 1.05 (s, 3H), 1.04 (s, 3H), 0.95 (s, 3H).

Compound 42: A mixture of N-tert-butoxycarbonyl-3-aminopropionitrile (858 mg, 5.04 mmol), hydroxylamine hydrochloride (1.39 g, 20.0 mmol) and triethylamine (4.2 mL, 30 mmol) in ethanol (20 mL) was heated to 80° C. with stirring in a sealed tube for 2 d. The resultant solution was diluted with EtOAc (300 mL) and washed with water/sat. aq NaHCO₃ (1:1, 100 mL) and brine (25 mL). The organic extract was dried with Na₂SO₄, filtered and concentrated to give compound 42 contaminated with N-tert-butoxycarbonyl-3-aminopropionitrile (73:27, 773.4 mg) as a crystalline solid that was used without further purification.

Compound 43: A mixture of compound 35 [synthesized from compound 34 (1.08 g, 2.20 mmol)], impure compound 42 (73:27—Int4:N-tert-butoxycarbonyl-3-aminopropionitrile, 773.4 mg) and triethylamine (1.23 mL, 8.83 mmol) in CH₂Cl₂ (18 mL) was stirred at room temperature for 2 h. The resultant mixture was diluted with EtOAc (100 mL); washed with water (25 mL), sat. aq. NaHCO₃ (25 mL) and brine (25 mL). The organic extract was dried with Na₂SO₄; filtered and concentrated. The residue was purified by column chromatography (silica gel, eluting with 0 to 10% MeOH in CH₂Cl₂) to give a mixture of compound 43 and 34 (1.01 g, -4:1-43:34) as a solid that was used without further purification.

Compound 44: A solution of impure compound 43 (˜4:1-43:34, 904 mg, ˜1.07 mmol) and tetrabutylammonium hydroxide (40% w/w in water, 3.6 mL, 5.5 mmol) in THF (14 mL) was stirred at room temperature under N₂ for 5 h. The resultant mixture was diluted with EtOAc (200 mL); washed with water (50 mL), sat. aq. NaHCO₃ (50 mL) and brine (50 mL). The organic extract was dried with Na₂SO₄, filtered and concentrated. The residue was purified by column chromatography (silica gel, eluting with 0 to 100% EtOAc in hexanes) to give compound 44 (578.2 mg, 45% yield from compound 34) as a solid. m/z=559.4 (M-Boc+2).

T16: A mixture of compound 44 (61 mg, 0.093 mmol) and HCl (12 M aq., 0.25 mL, 3.0 mmol) in MeOH (2.5 mL) was stirred at room temperature. Additional HCl (12 M aq., 0.75 mL, 9.0 mmol) was added after 3.5 h, and stirring continued at room temperature for a total of 24 h. The resultant mixture was diluted with EtOAc (50 mL) and washed with sat. aq. NaHCO₃ (25 mL). The organic extract was dried with Na₂SO₄, filtered and concentrated. The residue was purified by column chromatography (silica gel, eluting with 0 to 10% MeOH in CH₂Cl₂) to give compound T16 (43.1 mg, 83% yield) as an off-white solid. m/z=559.3 (M+1); ¹H NMR (400 MHz, CDCl₃) δ 8.02 (s, 1H), 5.95 (s, 1H), 3.23 (td, J=4.1, 13.9 Hz, 1H), 3.06 (m, 3H), 2.84 (t, J=6.5 Hz, 2H), 2.19 (dt, J=4.1, 13.8 Hz, 1H), 1.76 (m, 11H), 1.43 (s, 3H), 1.23 (s, 3H), 1.20 (m, 5H), 1.13 (s, 3H), 1.10 (s, 3H), 1.03 (s, 6H), 0.93 (s, 3H).

T17: Methyl chloroformate (12 μL, 0.16 mmol) was added to a solution of compound T16 (51.7 mg, 0.0926 mmol) and triethylamine (42 μL, 0.301 mmol) in CH₂Cl₂ (1 mL), and was stirred at room temperature for 2 h. Additional methyl chloroformate (30 μL, 0.39 mmol) was added, and stirring continued overnight. The mixture was diluted with CH₂Cl₂ (35 mL), and washed with HCl (1M aq., 15 mL) and brine (15 mL). The organic extract was dried with Na₂SO₄, filtered and concentrated. The residue was purified by column chromatography (silica gel, eluting with 0 to 100% EtOAc in hexanes) to give compound T17 (11.6 mg, 20% yield) as a white solid. m/z=617.4 (M+1); ¹H NMR (400 MHz, CDCl₃) δ 8.02 (s, 1H), 5.96 (s, 1H), 5.10 (br s, 1H), 3.64 (s, 3H), 3.57 (q, J=6.2 Hz, 2H), 3.23 (td, J=3.5, 13.4 Hz, 1H), 3.01 (d, J=4.7 Hz, 1H), 2.93 (t, J=6.4 Hz, 2H), 2.20 (dt, J=4.1, 13.8 Hz, 1H), 1.90 (m, 2H), 1.69 (m, 7H), 1.44 (s, 3H), 1.30 (m, 5H), 1.24 (s, 3H), 1.15 (s, 3H), 1.09 (s, 3H), 1.04 (s, 3H), 1.04 (s, 3H), 0.94 (s, 3H).

T18: Ethyl isocyanate (8 μL, 0.101 mmol) was added to a solution of compound T16 (55.8 mg, 0.0999 mmol) and triethylamine (0.14 mL, 1.0 mmol) in CH₂Cl₂ (1 mL), and was stirred at room temperature for 1 h. The resultant mixture was diluted with CH₂Cl₂ (35 mL), and washed with HCl (1M aq., 15 mL) and brine (15 mL). The organic extract was dried with Na₂SO₄, filtered and concentrated. The residue was purified by column chromatography (silica gel, eluting with 0 to 100% EtOAc in hexanes) to give compound T18 (42.1 mg, 67% yield) as a white solid. m/z=630.4 (M+1); ¹H NMR (400 MHz, CDCl₃) δ 8.02 (s, 1H), 5.96 (s, 1H), 4.90 (t, J=5.8 Hz, 1H), 4.23 (t, J=5.5 Hz, 1H), 3.56 (m, 2H), 3.24 (td, J=4.2, 14.4 Hz, 1H), 3.16 (ddq, J=1.9, 5.5, 7.2 Hz, 2H), 3.02 (d, J=4.7 Hz, 1H), 2.92 (m, 2H), 2.20 (dt, J=4.1, 13.8 Hz, 1H), 1.90 (m, 2H), 1.70 (m, 7H), 1.44 (s, 3H), 1.33 (m, 5H), 1.24 (s, 3H), 1.15 (s, 3H), 1.13 (t, J=7.2 Hz, 3H), 1.09 (s, 3H), 1.04 (s, 6H), 0.94 (s, 3H).

T19: Acetyl chloride (0.15 mL, 2.1 mmol) was added to a solution of compound T16 (45.7 mg, 0.0818 mmol) and triethylamine (0.11 mL, 0.79 mmol) in CH₂Cl₂ (1 mL), and the mixture was stirred at room temperature for 15 min. The resultant heterogeneous mixture was diluted with HCl (1M aq., 15 mL) and extracted with EtOAc (50 mL). The organic fraction was washed with brine (10 mL), dried with Na₂SO₄, filtered and concentrated. The residue was purified by column chromatography (silica gel, eluting with 0 to 15% MeOH in CH₂Cl₂) to give compound T19 (16.3 mg, 33% yield) as a white solid. m/z=601.4 (M+1); ¹H NMR (400 MHz, CDCl₃) δ 8.02 (s, 1H), 6.01 (br s, 1H), 5.97 (s, 1H), 3.62 (m, 2H), 3.24 (td, J=4.2, 13.6 Hz, 1H), 3.01 (d, J=4.7 Hz, 1H), 2.93 (t, J=6.3 Hz, 2H), 2.21 (m, 1H), 1.95 (s, 3H), 1.77 (m, 9H), 1.45 (s, 3H), 1.24 (s, 3H), 1.22 (m, 5H), 1.15 (s, 3H), 1.10 (s, 3H), 1.05 (s, 3H), 1.04 (s, 3H), 0.95 (s, 3H).

Compound 45: Compound 5 (40 mg, 0.079 mmol) in AcOH (1 mL) was treated with acetic anhydride (11 μL, 0.12 mmol) at room temperature. The mixture was stirred at room temperature for 30 min, and then heated at 100° C. for 2 h. The mixture was cooled to room temperature, diluted with toluene (10 mL), and concentrated. The residue was purified by column chromatography (silica gel, eluting with 0-40% EtOAc in hexanes) to give compound 45 (40 mg, 95% yield) as a white foam. m/z=532 (M+1).

Compound 46: Compound 45 (40 mg, 0.075 mmol) was mixed with K₂CO₃ (44 mg, 0.32 mmol) and MeOH (2 mL). The mixture was stirred at room temperature under N₂ for 14 h; treated with 10% NaH₂PO₄ (20 mL); and extracted with EtOAc (2×20 mL). The combined organic extracts were dried with Na₂SO₄, filtered, and concentrated. The residue was combined with crude product obtained from compound 46 (16 mg, 0.030 mmol), and purified by column chromatography (silica gel, eluting with 0-40% EtOAc in hexanes) to give compound 46 (43.5 mg, 78% yield) as a white solid. m/z=532 (M+1).

T20: Compound 46 (43.5 mg, 0.082 mmol) and 1,3-dibromo-5,5-dimethylhydantoin (13 mg, 0.045 mmol) were mixed with anhydrous DMF (0.8 mL) at 0° C. under N₂. The mixture was stirred at 0° C. for 1 h, and treated with pyridine (20 μL, 0.25 mmol). The mixture was heated at 55° C. for 6 h; cooled to room temperature; diluted with EtOAc (25 mL); and washed with 1 N aq. HCl (10 mL) and water (2×15 mL). The organic extract was dried with Na₂SO₄, filtered and concentrated. The residue was purified by column chromatography (silica gel, eluting with 0-35% EtOAc in hexanes) to give compound T20 (34 mg, 78% yield) as a white solid. m/z=530 (M+1); ¹H NMR (400 MHz, CDCl₃) δ 8.02 (s, 1H), 5.95 (s, 1H), 3.20 (m, 1H), 3.01 (d, J=4.7 Hz, 1H), 2.57 (s, 3H), 2.16 (td, J=13.5, 4.2 Hz, 1H), 1.44 (s, 3H), 1.24 (s, 3H), 1.15 (s, 6H), 1.12-1.93 (m, 14H), 1.05 (s, 3H), 1.04 (s, 3H), 0.94 (s, 3H).

Compound 47: A solution of compound 25 (150 mg, 0.30 mmol) in THF (2.4 mL) and MeOH (0.6 mL) was cooled to 0° C. (Trimethylsilyl)diazomethane (2 M in hexanes solution, 183 μL, 0.366 mmol) was added. The mixture was stirred at 0° C. for 20 min; quenched with acetic acid; diluted with toluene; and concentrated. The residue was combined with the crude product obtained from compound 25 (50 mg, 0.10 mmol), and purified by column chromatography (silica gel, eluting with 0 to 35% acetone in hexanes) to give compound 47 (139 mg, 90% yield) as a white solid. m/z=507 (M+1).

Compound 48: A mixture compound 47 (137 mg, 0.27 mmol) in ethyl formate (0.65 mL, 8.08 mmol) was cooled to 0° C. under N₂ and treated dropwise with sodium methoxide solution (4.37 M in methanol, 0.62 mL, 2.71 mmol). The reaction mixture was stirred at 0° C. for 1 h, and then treated with 6 N aq. HCl (0.45 mL, 2.7 mmol), followed by EtOH (2.7 mL) and hydroxylamine hydrochloride (28 mg, 0.40 mmol). The reaction mixture was heated at 60° C. for 6 h; cooled to room temperature; diluted with EtOAc; and washed with water. The organic extract was dried with Na₂SO₄; filtered; and concentrated. The residue was purified by column chromatography (silica gel, eluting with 0 to 50% EtOAc in hexanes) to give compound 48 (112 mg, 78% yield) as a white solid. m/z=532 (M+1).

Compound 49: To a mixture of compound 48 (110 mg, 0.21 mmol) in anhydrous MeOH (2 mL) was added NaOMe (4.37 M in MeOH, 95 μL, 0.42 mmol) under N₂. The mixture was heated at 45° C. for 1 to 2 h, and then cooled to room temperature. The mixture was diluted with 10% aq. NaH₂PO₄ and extracted twice with EtOAc. The combined organic extracts were dried with Na₂SO₄, filtered and concentrated. The residue was purified by column chromatography (silica gel, eluting with 0 to 50% EtOAc in hexanes) to give compound 49 (86 mg, 77% yield) as a white solid. m/z=532 (M+1).

T21: Under N₂, compound 49 (84 mg, 0.16 mmol) was dissolved in anhydrous DMF (0.4 mL), and cooled to 0° C. A solution of 1,3-dibromo-5,5-dimethylhydantoin (23 mg, 0.080 mmol) in DMF (0.4 mL) was added. The mixture was stirred at 0° C. for 2 h. Pyridine (40 μL, 0.50 mmol) was added. The mixture was heated at 55° C. for 6 h, and then cooled to room temperature. The mixture was diluted with EtOAc and washed with 1 N aqueous HCl and water (3×) sequentially. The organic extract was dried with Na₂SO₄, filtered and concentrated. The residue was purified by column chromatography (silica gel, eluting with 0 to 50% EtOAc in hexanes) to give compound T21 (65 mg, 78% yield) as a white solid. m/z=530 (M+1); ¹H NMR (400 MHz, CDCl₃) δ 8.02 (s, 1H), 5.94 (s, 1H), 4.31 (s, 3H), 3.30 (m, 1H), 2.97 (d, J=4.7 Hz, 1H), 2.23 (td, J=13.6, 4.2 Hz, 1H), 2.02 (m, 1H), 1.83 (td, J=13.9, 4.5 Hz, 1H), 1.59-1.77 (m, 7H), 1.42 (s, 3H), 1.38-1.49 (m, 3H), 1.24 (s, 3H), 1.14-1.30 (m, 2H), 1.14 (s, 3H), 1.06 (s, 3H), 1.05 (s, 3H), 1.00 (s, 3H), 0.95 (s, 3H).

A stock solution of compound 35 (≤0.113 M) and trimethylamine (0.453 M) in CH₂Cl₂ was prepared by dissolving compound 35 (prepared from 28.92 mmol of compound 34) and triethylamine (16.1 mL, 116 mmol) in CH₂Cl₂ (230 mL). The total volume of the solution was 256 mL. The stock solution is used for the synthesis of compound 50 and 52.

Compound 50: 2,2,2-Trifluoro-N′-hydroxy-ethanimidamide (229.2 mg, 1.79 mmol) was added to the stock solution of compound 35 and Et₃N in CH₂Cl₂ [26.1 mL, containing compound 35 (2.95 mmol) and Et₃N (11.8 mmol)], and the mixture stirred overnight at room temperature. The resultant mixture was diluted with EtOAc; washed with water, sat. aq. NaHCO₃, and brine. The organic extract was dried with Na₂SO₄; filtered and concentrated. The residue was purified by column chromatography (silica gel, eluting with 0 to 10% MeOH in CH₂Cl₂) to give impure compound 50 (965.7 mg) as a solid that was used without further purification. m/z=602.3 (M+1).

T22: A mixture of impure compound 50 (353 mg) and tetrabutylammonium hydroxide (40% w/w in water, 1.25 mL, 1.9 mmol) in THF (5 mL) was stirred at room temperature under N₂ overnight. The resultant mixture was diluted with EtOAc; washed with water, sat. aq. NaHCO₃ and brine. The organic extract was dried with Na₂SO₄, filtered and concentrated. The residue was purified by column chromatography (silica gel, eluting with 0 to 100% EtOAc in hexanes) to give compound T22 (63.3 mg, 10% yield from compound 34) as a white solid. m/z=584.3 (M+1); ¹H NMR (400 MHz, CDCl₃) δ 8.02 (s, 1H), 5.98 (s, 1H), 3.26 (td, J=4.1, 14.1 Hz, 1H), 2.99 (d, J=4.7 Hz, 1H), 2.27 (dt, J=4.2, 13.9 Hz, 1H), 1.96 (m, 2H), 1.74 (m, 6H), 1.61 (dt, J=4.4, 13.9 Hz, 1H), 1.46 (s, 3H), 1.36 (s, 5H), 1.25 (s, 3H), 1.16 (s, 3H), 1.11 (s, 3H), 1.06 (s, 3H), 1.06 (s, 3H), 0.96 (s, 3H).

Compound 51: Propionamidoxime (250 mg, 2.84 mmol) was added to the stock solution of compound 35 and Et₃N in CH₂Cl₂ [30 mL, containing compound 35 (3.38 mmol) and Et₃N (13.5 mmol)], and the mixture stirred overnight at room temperature. The resultant mixture was diluted with EtOAc; washed with water, sat. aq. NaHCO₃, and brine. The organic extract was dried with Na₂SO₄; filtered and concentrated. The residue was purified by column chromatography (silica gel, eluting with 0 to 10% MeOH in CH₂Cl₂) to give impure compound 51 (1.0959 g) that was used without further purification. m/z=562.3 (M+1).

T23: A mixture of impure compound 51 (346.2 mg) and tetrabutylammonium hydroxide (40% w/w in water, 1.25 mL, 1.9 mmol) in THF (5 mL) was stirred at room temperature under N₂ overnight. The resultant mixture was diluted with EtOAc; washed with water, sat. aq. NaHCO₃ and brine. The organic extract was dried with Na₂SO₄, filtered and concentrated. The residue was purified by column chromatography (silica gel, eluting with 0 to 100% EtOAc in hexanes) to give compound T23 (209.9 mg, 36% yield from compound 34) as a white solid. m/z=544.3 (M+1); ¹H NMR (400 MHz, CDCl₃) δ 8.02 (s, 1H), 5.96 (s, 1H), 3.24 (dt, J=13.1, 4.3 Hz, 1H), 3.11 (d, J=4.7 Hz, 1H), 2.73 (qd, J=7.6, 1.5 Hz, 2H), 2.20 (td, J=13.8, 4.1 Hz, 1H), 1.45 (s, 3H), 1.29 (t, J=7.6 Hz, 3H), 1.24 (s, 3H), 1.18-1.97 (m, 14H), 1.15 (s, 3H), 1.11 (s, 3H), 1.05 (s, 3H), 1.04 (s, 3H), 0.94 (s, 3H).

Compound 52a: Compound 5 (84 mg, 0.17 mmol) in CH₂Cl₂ (6 mL) was cooled to 0° C. Et₃N (46 μL, 0.33 mmol) and propionyl chloride (23 mg, 0.25 mmol) in CH₂Cl₂ (1 mL) were added sequentially. After stirring at 0° C. for 1 h, the mixture was treated with sat. aq. NaHCO₃ (5 mL); stirred for 5 min; and extracted with CH₂Cl₂ (3×10 mL). The combined organic extracts were dried with Na₂SO₄, filtered and concentrated. The residue was purified by column chromatography (silica gel, eluting with 0-60% EtOAc in hexanes) to give compound 52a (83 mg, 89% yield) as a white solid. m/z=564.3 (M+1)

Compound 53a: Compound 52a (83 mg, 0.15 mmol) in AcOH (1 mL) was heated at 100° C. for 1 h. The mixture was cooled to room temperature; diluted with toluene (15 mL); and concentrated. The residue was diluted with toluene (10 mL) and concentrated again. The residue was purified by column chromatography (silica gel, eluting with 0-40% EtOAc in hexanes) to give compound 53a (67 mg, 77% yield) as a white solid. m/z=546.3 (M+1).

Compound 54a: Compound 53a (67 mg, 0.12 mmol) in MeOH (1.2 mL) was treated with sodium methoxide (25 wt. % in MeOH, 66 mg, 0.31 mmol) at room temperature. The mixture was stirred at 55° C. for 1 h. After cooled to room temperature, the mixture was treated with 10% NaH₂PO₄ (5 mL); and extracted with EtOAc (2×15 mL). The combined organic extracts were dried with Na₂SO₄, filtered, and concentrated. The residue was purified by column chromatography (silica gel, eluting with 0-45% EtOAc in hexanes) to give compound 54a (65 mg, 97% yield) as a white solid. m/z=546.3 (M+1).

T24: Compound 54a (65 mg, 0.12 mmol) and 1,3-dibromo-5,5-dimethylhydantoin (18.7 mg, 0.066 mmol) were mixed with anhydrous DMF (0.6 mL) at 0° C. under N₂. The mixture was stirred at 0° C. for 1 h, and treated with pyridine (38 μL, 0.48 mmol). The mixture was heated at 55° C. for 2 h, and at 60° C. for 4 h; cooled to room temperature; diluted with EtOAc (25 mL); and washed with 1 N aq. HCl (10 mL) and water (2×15 mL). The organic extract was dried with Na₂SO₄, filtered and concentrated. The residue was purified by column chromatography (silica gel, eluting with 0-45% EtOAc in hexanes) to give compound T24 (49 mg, 76% yield) as a white solid. m/z=544.3 (M+1); ¹H NMR (400 MHz, CDCl₃) δ 8.03 (s, 1H), 5.95 (s, 1H), 3.21 (dt, J=13.7, 4.3 Hz, 1H), 3.03 (d, J=4.7 Hz, 1H), 2.89 (q, J=7.6 Hz, 2H), 2.16 (td, J=13.7, 4.2 Hz, 1H), 1.44 (s, 3H), 1.36 (t, J=7.6 Hz, 3H), 1.24 (s, 3H), 1.15 (s, 3H), 1.14 (s, 3H), 1.10-1.95 (m, 14H), 1.05 (s, 3H), 1.04 (s, 3H), 0.94 (s, 3H).

Compound 52b: Using the same procedure as described for the synthesis of compound 52a, compound 52b (white solid, 96 mg, 82% yield) was synthesized from compound 5 (103 mg, 0.20 mmol). m/z=576 (M+1).

Compound 53b: Using the same procedure as described for the synthesis of compound 53a, compound 53b (solid, 67 mg, 72% yield) was synthesized from compound 52b (96 mg, 0.17 mmol). n/z=558 (M+1).

Compound 54b: Using the same procedure as described for the synthesis of compound 54a, compound 54b (white solid, 58 mg, 89% yield) was synthesized from compound 53b (65 mg, 0.12 mmol). m/z=558 (M+1).

T25: Using the same procedure as described for the synthesis of compound T24, compound T25 (white solid, 44 mg, 76% yield) was synthesized from compound 54b (58 mg, 0.10 mmol). m/z 25=556 (M+1); ¹H NMR (400 MHz, CDCl₃) δ 8.03 (s, 1H), 5.94 (s, 1H), 3.17 (dt, J=13.7, 4.2 Hz, 1H), 3.02 (d, J=4.7 Hz, 1H), 2.15 (m, 2H), 1.44 (s, 3H), 1.24 (s, 3H), 1.15 (s, 6H), 1.11-1.92 (m, 18H), 1.03 (s, 6H), 0.93 (s, 3H).

Compound 55a: Using the same procedure as described for the synthesis of compound 51, impure compound 55a (1.3624 g) was synthesized from compound 35 (3.67 mmol), n-butyramidoxime (234.6 mg, 2.30 mmol) and trimethylamine (14.6 mmol). m/z=576 (M+1).

T26: Using the same procedure as described for the synthesis of compound T23, compound T26 (white solid, 159.3 mg, 47% yield from 35) was synthesized from compound 55a (361.1 mg) and tetrabutylammonium hydroxide (40% w/w in water, 1.25 mL, 1.9 mmol). m/z=558 (M+1); ¹H NMR (400 MHz, CDCl₃) δ 8.02 (s, 1H), 5.96 (s, 1H), 3.25 (m, 1H), 3.08 (d, J=4.7 Hz, 1H), 2.68 (t, J=7.5 Hz, 2H), 2.20 (td, J=13.8, 4.1 Hz, 1H), 1.90 (m, 2H), 1.45 (s, 3H), 1.24 (s, 3H), 1.17-1.81 (m, 14H), 1.15 (s, 3H), 1.10 (s, 3H), 1.05 (s, 3H), 1.04 (s, 3H), 0.95 (t, J=7.4 Hz, 3H), 0.94 (s, 3H).

Compound 55b: Using the same procedure as described for the synthesis of compound 51, impure compound 55b (1.1737 g) was synthesized from compound 35 (3.67 mmol), isobutyramidoxime (255.1 mg, 2.50 mmol) and trimethylamine (14.6 mmol). m/z=576 (M+1).

T27: Using the same procedure as described for the synthesis of compound T23, compound T27 (white solid, 240.3 mg, 57% yield from 35) was synthesized from compound 55b (354.1 mg) and tetrabutylammonium hydroxide (40% w/w in water, 1.25 mL, 1.9 mmol). m/z=558 (M+1); ¹H NMR (400 MHz, CDCl₃) δ 8.02 (s, 1H), 5.96 (s, 1H), 3.22 (m, 1H), 3.18 (d, J=4.8 Hz, 1H), 3.07 (hept, J=6.9 Hz, 1H), 2.19 (td, J=13.8, 4.1 Hz, 1H), 1.90 (td, J=13.7, 5.4 Hz, 2H), 1.45 (s, 3H), 1.30 (d, J=6.9 Hz, 3H), 1.30 (d, J=6.9 Hz, 3H), 1.24 (s, 3H), 1.16-1.79 (m, 12H), 1.15 (s, 3H), 1.11 (s, 3H), 1.05 (s, 3H), 1.04 (s, 3H), 0.94 (s, 3H).

Compound 55c: Using the same procedure as described for the synthesis of compound 51, impure compound 55c (1.4948 g) was synthesized from compound 35 (3.13 mmol), N-hydroxy-2,2-dimethylpropanimidamide (258.2 mg, 2.22 mmol) and trimethylamine (12.5 mmol). m/z=590 (M+1).

T28: Using the same procedure as described for the synthesis of compound T23, compound T28 (white solid, 157.2 mg, 52% yield from 35) was synthesized from compound 55c (357.7 mg) and tetrabutylammonium hydroxide (40% w/w in water, 1.25 mL, 1.9 mmol). m/z=572 (M+1); ¹H NMR (400 MHz, CDCl₃) δ 8.02 (s, 1H), 5.96 (s, 1H), 3.23 (m, 2H), 2.19 (td, J=13.7, 4.0 Hz, 1H), 1.90 (m, 2H), 1.47 (m, 12H), 1.45 (s, 3H), 1.33 (s, 9H), 1.24 (s, 3H), 1.15 (s, 3H), 1.11 (s, 3H), 1.04 (s, 3H), 1.03 (s, 3H), 0.94 (s, 3H).

Compound 55d: Using the same procedure as described for the synthesis of compound 51, impure compound 55d (279.1 mg) was synthesized from compound 35 (0.64 mmol), N′ hydroxycyclopropanecarboximidamide (84.3 mg, 0.842 mmol) and trimethylamine (2.6 mmol). m/z=574 (M+1).

T29: Using the same procedure as described for the synthesis of compound T23, compound T29 (white solid, 129.3 mg, 36% yield from 35) was synthesized from compound 55d (279.1 mg) and tetrabutylammonium hydroxide (40% w/w in water, 1 mL, 1.5 mmol). m/z=556 (M+1); ¹H NMR (400 MHz, CDCl₃) δ 8.02 (s, 1H), 5.96 (s, 1H), 3.17 (m, 1H), 3.12 (d, J=4.8 Hz, 1H), 2.17 (td, J=13.8, 4.1 Hz, 1H), 2.06 (m, 1H), 1.87 (m, 2H), 1.46 (s, 3H), 1.24 (s, 3H), 1.15 (s, 3H), 1.12 (s, 3H), 1.02 (s, 6H), 0.93 (s, 3H). 0.88-1.80 (m, 16H).

Compound 55e: Using the same procedure as described for the synthesis of compound 51, impure compound 55e (514.3 mg) was synthesized from compound 35 (1.33 mmol), 2-cyclopropyl-N′-hydroxyethanimidamide (101.3 mg, 0.887 mmol) and trimethylamine (5.31 mmol). m/z=588 (M+1).

T30: Using the same procedure as described for the synthesis of compound T23, compound T30 (white solid, 176.0 mg, 52% yield from 35) was synthesized from compound 55e (344.0 mg) and tetrabutylammonium hydroxide (40% w/w in water, 1.25 mL, 1.9 mmol). m/z=570 (M+1); ¹H NMR (400 MHz, CDCl₃) δ 8.02 (s, 1H), 5.96 (s, 1H), 3.24 (m, 1H), 3.12 (d, J=4.7 Hz, 1H), 2.61 (m, 2H), 2.20 (td, J=13.8, 4.1 Hz, 1H), 1.91 (m, 2H), 1.45 (s, 3H), 1.24 (s, 3H), 1.15 (s, 3H), 1.12 (s, 3H), 1.07-1.79 (m, 13H), 1.05 (s, 3H), 1.04 (s, 3H), 0.95 (s, 3H), 0.50 (m, 2H), 0.24 (m, 2H).

Compound 55f: Using the same procedure as described for the synthesis of compound 51, impure compound 55f (239.0 mg) was synthesized from compound 35 (0.64 mmol), N′-hydroxycyclobutanecarboxinidamide (89.8 mg, 0.787 mmol) and trimethylamine (2.6 mmol). m/z=588 (M+1).

T31: Using the same procedure as described for the synthesis of compound T23, compound T31 (white solid, 129.9 mg, 36% yield from 35) was synthesized from compound 55f (239.0 mg) and tetrabutylammonium hydroxide (40% w/w in water, 0.9 mL, 1.35 mmol). m/z=570 (M+1); ¹H NMR (400 MHz, CDCl₃) δ 8.03 (s, 1H), 5.97 (s, 1H), 3.63 (m, 1H), 3.21 (m, 2H), 2.33 (m, 4H), 2.20 (td, J=13.9, 4.1 Hz, 1H), 1.45 (s, 3H), 1.24 (s, 3H), 1.17-2.14 (m, 16H), 1.15 (s, 3H), 1.11 (s, 3H), 1.05 (s, 3H), 1.04 (s, 3H), 0.94 (s, 3H).

Compound 55g: Using the same procedure as described for the synthesis of compound 51, compound 55g (115.6 mg) was synthesized from compound 35 (0.64 mmol), N-hydroxycyclopentanecarboxinidamide (97.7 mg, 0.762 mmol) and trimethylamine (2.6 mmol). m/z=602 (M+1).

T32: Using the same procedure as described for the synthesis of compound T23, compound T32 (white solid, 60.9 mg, 16% yield from 35) was synthesized from compound 55g (115.6 mg, 0.192 mmol) and tetrabutylammonium hydroxide (40% w/w in water, 0.4 mL, 0.6 mmol). m/z=584 (M+1); ¹H NMR (400 MHz, CDCl₃) δ 8.02 (s, 1H), 5.96 (s, 1H), 3.19 (m, 3H), 2.19 (td, J=13.8, 4.1 Hz, 1H), 1.45 (s, 3H), 1.24 (s, 3H), 1.17-2.07 (m, 22H), 1.15 (s, 3H), 1.11 (s, 3H), 1.04 (s, 3H), 1.03 (s, 3H), 0.94 (s, 3H).

Compound 55h: Using the same procedure as described for the synthesis of compound 51, impure compound 55h (544.9 mg) was synthesized from compound 35 (1.06 mmol), N′-hydroxycyclohexanrecarboximidamide (99.5 mg, 0.700 mmol) and trimethylamine (4.2 mmol). m/z=616 (M+1).

T33: Using the same procedure as described for the synthesis of compound T23, compound T33 (white solid, 149.8 mg, 56% yield from 35) was synthesized from compound 55h (346.1 mg) and tetrabutylammonium hydroxide (40% w/w in water, 1.25 mL, 1.9 mmol). m/z=598 (M+1); ¹H NMR (400 MHz, CDCl₃) δ 8.02 (s, 1H), 5.96 (s, 1H), 3.22 (m, 1H), 3.14 (d, J=4.7 Hz, 1H), 2.77 (tt, J=11.4, 3.6 Hz, 1H), 2.19 (td, J=13.8, 4.1 Hz, 1H), 1.58 (m, 24H), 1.45 (s, 3H), 1.24 (s, 3H), 1.15 (s, 3H), 1.10 (s, 3H), 1.05 (s, 3H), 1.03 (s, 3H), 0.94 (s, 3H).

Compound 58: Compound 56 (0.86 g, 1.9 mmol) in CH₂Cl₂ (19 mL) was treated with oxalyl chloride (0.5 mL, 5.7 mmol) and DMF (15 μL, 0.19 mmol) at 0° C. sequentially. The reaction was stirred at room temperature for 2 h, and then concentrated. The residue was dissolved in toluene (3×20 mL) and concentrated to give the compound 57 as a yellow solid. Compound 57 was dissolved in CH₂Cl₂ (25 mL) and cooled to 0° C. Et₃N (1.1 mL, 7.6 mmol) was added, followed by the addition of 2-fluoro-N-hydroxyethanimidamide (0.26 g, 2.8 mmol). The reaction was stirred at room temperature for 2 h. The reaction mixture was then concentrated and partitioned between EtOAc (30 mL) and water (20 mL). The layers were separated; and the organic layer was washed with water (2×20 mL). The aqueous washes were extracted with EtOAc (20 mL). The combined organic extracts were washed with brine (20 mL), dried with Na₂SO₄, filtered, and concentrated. The residue was purified by column chromatography (silica gel, eluting with 0-100% EtOAc in CH₂Cl₂) to give compound 58 (0.87 g, 87% yield) as a white solid. m/z=529 (M+1).

Compound 59: Compound 58 (20 mg, 0.038 mmol) was dissolved in o-xylene (0.5 mL) and the reaction was heated at 180° C. for 14 h in a sealed-tube. The reaction mixture was concentrated, and the residue was purified by column chromatography (silica gel, eluting with 0-100% EtOAc in hexane) to give compound 59 (9 mg, 49% yield) as a white solid. m/z=511 (M+1).

Compound 60: Compound 59 (233 mg, 0.46 mmol) was dissolved in ethyl formate (3.3 mL, 41 mmol) and cooled to 0° C. Sodium methoxide solution (25 wt. % in MeOH, 1 mL, 4.56 mmol) was added under N₂. After stirring at room temperature for 1.5 h, the reaction mixture was cooled to 0° C. HCl (12 N aqueous solution, 0.8 mL, 4.56 mmol) was added, followed by the addition of EtOH (5 mL) and hydroxylamine hydrochloride (47.6 mg, 0.68 mmol). The reaction was heated at 60° C. for 4 h, and then cooled to room temperature. The reaction mixture was diluted with EtOAc (30 mL); washed with water (2×20 mL) and sat. aq. NaHCO₃ (20 mL). The aqueous washes were extracted with EtOAc (20 mL). The combined organic extracts were washed with brine (20 mL), dried with Na₂SO₄, filtered, and concentrated. The residue was purified by column chromatography (silica gel, eluting with 0-100% EtOAc in hexane) to give compound 60 (265 mg, quantitative yield) as an off-white solid. m/z=536 (M+1).

Compound 61: Compound 60 (265 mg, 0.49 mmol) in MeOH (5 mL) was treated with sodium methoxide (25 wt. % in MeOH, 226 μL, 0.99 mmol) at room temperature. The reaction was heated at 55° C. for 1.5 h, and then cooled to 0° C. 10% aq. NaH₂PO₄ (10 mL) was added and the mixture was extracted with EtOAc (2×20 mL). The combined organic extracts were washed with brine, dried with Na₂SO₄, filtered and concentrated. The residue was purified by column chromatography (silica gel, eluting with 0-100% EtOAc in hexane) to give compound 61 (217 mg, 82% yield) as a white solid. m/z=536 (M+1).

T34: Compound 61 (217 mg, 0.41 mmol) was dissolved in DMF (1 mL) and cooled to 0° C. under N₂. 1,3-Dibromo-5,5-dimethylhydantoin (58 mg, 0.20 mmol) in DMF (1 mL) was added dropwise. The mixture was stirred at 0° C. for 2 h. Pyridine (98 μL, 1.21 mmol) was then added. The reaction was heated at 60° C. for 4 h. After cooled to room temperature, the mixture was diluted with EtOAc (20 mL) and washed with 1 N aq. HCl (10 mL), water (2×15 mL) and brine (10 mL). The organic extract was dried with Na₂SO₄, filtered, and concentrated. The residue was purified by column chromatography (silica gel, eluting with 0-100% EtOAc in hexane) to give compound T34 (168 mg, 78% yield) as a white solid. m/z=534 (M+1); ¹H NMR (400 MHz, CDCl₃) δ 8.00 (s, 1H), 6.02 (s, 1H), 5.44 (d, J=46.5 Hz, 2H), 3.27 (dt, J=13.9, 4.6 Hz, 1H), 3.05 (d, J=4.7 Hz, 1H), 2.45 (dq, J=6.7, 13.4 Hz, 1H), 2.23 (td, J=13.8, 4.1 Hz, 1H), 1.57 (s, 3H), 1.41 (s, 3H), 1.24 (d, J=6.7 Hz, 3H), 1.21-2.00 (m, 11H), 1.11 (s, 3H), 1.06 (s, 3H), 1.05 (s, 3H), 0.95 (s, 3H).

Compound 62: Compound 56 (500 mg, 1.02 mmol) in CH₂Cl₂ (14 mL) was treated with oxalyl chloride (275 μL, 3.14 mmol) and N,N-dimethylformamide (8 μL, 0.1 mmol) sequentially at 0° C. The reaction was stirred at room temperature for 2 h and then concentrated. The residue was dissolved in toluene (3×20 mL) and concentrated to give the acid chloride as a yellow solid. The acid chloride was dissolved in CH₂Cl₂ (14 mL) and cooled to 0° C. Triethylamine (0.58 mL, 4.16 mmol) and 2,2-difluoro-N′-hydroxyethanimidamide (173 mg, 1.57 mmol) were added. The reaction was stirred at room temperature for overnight. LC-MS indicates that the reaction was complete. The reaction was concentrated, and the residue was partitioned between ethyl acetate (20 mL) and sat. aq. NaHCO₃ (10 mL). The organic layer was separated and washed with sat. aq. NaHCO₃ (10 mL). The combined aqueous washes were extracted with ethyl acetate (20 mL). The combined organic extracts were washed with brine, dried with Na₂SO₄, filtered and concentrated. The residue was purified by column chromatography (silica gel, eluting with 0-100% EtOAc in hexanes) to give compound 62 (337 mg, 57% yield). m/z=570 (M+1).

T35: To a solution of compound 62 (100 mg, 0.176 mmol) THF (4 mL) at 0° C., was added tetrabutylammonium hydroxide (40 wt. % in water, 0.36 mL, 0.55 mmol) dropwise. The reaction was stirred at room temperature for overnight. LC-MS indicates that the reaction was complete. The mixture was partitioned between ethyl acetate (20 mL) and water (20 mL). The organic layer was separated and washed with water (2×10 mL). The combined aqueous washes were extracted with ethyl acetate (20 mL). The combined organic extracts were washed with brine (20 mL), dried with Na₂SO₄, filtered and concentrated. The residue was purified by column chromatography (silica gel, eluting with 0-100% EtOAc in hexanes) to give compound T35 (15.7 mg, 16% yield). m/z=552 (M+1); ¹H NMR (400 MHz, CDCl₃) δ 8.00 (s, 1H), 6.75 (t, J=52.5 Hz, 1H), 6.02 (s, 1H), 3.26 (dd, J=13.5, 3.5 Hz, 1H), 3.02 (d, J=4.7 Hz, 1H), 2.45 (dt, J=13.3, 6.7 Hz, 1H), 2.25 (td, J=13.9, 4.2 Hz, 1H), 2.01-1.20 (m, 14H), 1.41 (s, 3H), 1.24 (d, J=6.8 Hz, 3H), 1.11 (s, 3H), 1.06 (s, 3H), 1.05 (s, 3H), 0.96 (s, 3H).

Compound 63: To a solution of compound 56 (1.21 g, 2.46 mmol) in CH₂Cl₂ (50 mL) at 0° C. were added oxalyl chloride (0.65 mL, 7.43 mmol) and N,N-dimethylformamide (20 μL, 0.26 mmol) sequentially. The mixture was stirred at 0° C. for 20 h, and then concentrated in vacuo to give compound 63 (1.48 g, quantitative yield) as a yellow foamy solid, which was used in next step without further purification.

Compound 64: Triethylamine (0.79 mL, 5.67 mmol) was added slowly to a solution of compound 63 (82 wt. %, 860 mg, 1.42 mmol) in CH₂Cl₂ (15 mL) at room temperature under N₂. A solution of 2,2,2-Trifluoro-N′-hydroxy-ethanimidamide (181 mg, 1.42 mmol) in CH₂Cl₂ (6 mL) was then added dropwise. The reaction mixture was stirred at room temperature for 16 h, and then concentrated. The residue was diluted with EtOAc (100 mL). The mixture was washed with sat. aq. NaHCO₃ (30 mL) and brine (30 mL). The organic extract was dried with Na₂SO₄, filtered and concentrated. The residue was purified by column chromatography (silica gel, eluting with 0-10% MeOH in CH₂Cl₂) to give partially purified compound 64 (615 mg) as an orange foamy solid, which was used in next step without further purification. m/z=588.2 (M+1).

T36: A mixture of compound 64 (615 mg, ≤1.04 mmol) and tetrabutylammonium hydroxide (40 wt. % in water, 2.16 mL, 3.31 mmol) in THF (20 mL) was stirred at room temperature for 20 h under N₂. The reaction mixture was concentrated. The residue was diluted with EtOAc (40 mL). The mixture was washed with water (20 mL). The aqueous phase was separated and extracted with EtOAc (2×20 mL). The combined organic extracts were washed with brine (20 mL), dried with Na₂SO₄, filtered and concentrated. The residue was purified by column chromatography (silica gel, eluting with 0-50% EtOAc in hexanes) to give compound T36 (72.3 mg, 9% yield from compound 63) as white solid. m/z=570.2 (M+1); ¹H NMR (400 MHz, CDCl₃) δ 8.00 (s, 1H), 6.03 (s, 1H), 3.30-3.22 (m, 1H), 3.01 (d, J=4.7 Hz, 1H), 2.46 (dt, J=13.3, 6.7 Hz, 1H), 2.27 (td, J=13.9, 4.2 Hz, 1H), 2.02-1.20 (m, 14H), 1.42 (s, 3H), 1.24 (d, J=6.8 Hz, 3H), 1.11 (s, 3H), 1.07 (s, 3H), 1.06 (s, 3H), 0.96 (s, 3H).

Compound 65: Triethylamine (0.579 mL, 4.15 mmol) was added slowly to a solution of compound 63 (83 wt. %, 620 mg, 1.04 mmol) in CH₂Cl₂ (10 mL) at room temperature under N₂. Then a solution of N′-hydroxypropanimidamide (92 mg, 1.04 mmol) in CH₂Cl₂ (6 mL) was added dropwise. The mixture was stirred at room temperature for 16 h, and then concentrated. The residue was diluted with EtOAc (100 mL). The resultant mixture was washed with sat. aq. NaHCO₃ (30 mL) and brine (30 mL). The organic extract was dried with Na₂SO₄, filtered and concentrated. The residue was purified by column chromatography (silica gel, eluting with 0-10% MeOH in CH₂Cl₂) to give partially purified compound 65 (520 mg) as a yellow solid, which was used in next step without further purification. m/z=548.3 (M+1).

T37: A mixture of compound 65 (520 mg, 0.95 mmol) and tetrabutylammonium hydroxide (40% w/w in water, 1.98 mL, 3.04 mmol) in THF (15 mL) was stirred at room temperature for 15 h under N₂. The reaction mixture was concentrated. The residue was diluted with EtOAc (30 mL) and washed with water (20 mL). The aqueous phase was separated and extracted with EtOAc (2×20 mL). The combined organic extracts were washed with brine (20 mL), dried with Na₂SO₄, filtered and concentrated. The residue was purified by column chromatography (silica gel, eluting with 0-50% EtOAc in hexanes) to give compound T37 (267 mg, 48% yield from compound 63) as white solid. m/z=530.3 (M+1); ¹H NMR (400 MHz, CDCl₃) δ 8.01 (s, 1H), 6.02 (s, 1H), 3.28-3.20 (m, 1H), 3.13 (d, J=4.7 Hz, 1H), 2.73 (qd, J=7.6, 1.6 Hz, 2H), 2.45 (dt, J=13.3, 6.7 Hz, 1H), 2.20 (td, J=13.8, 4.2 Hz, 1H), 2.02-1.18 (m, 14H), 1.41 (s, 3H), 1.29 (t, J=7.6 Hz, 3H), 1.24 (d, J=6.7 Hz, 3H), 1.11 (s, 3H), 1.05 (s, 3H), 1.04 (s, 3H), 0.94 (s, 3H).

Compound 66: Compound 65 (260 mg, 0.524 mmol) in CH₂Cl₂ (5 mL) was cooled to 0° C. under N₂. Hydrazine hydrate (50 wt. %, 98 μL, 1.57 mmol) was added dropwise. The mixture was stirred at room temperature for 10 min; diluted with CH₂Cl₂ (10 mL); and washed with water (15 mL). The aqueous wash was extracted with CH₂Cl₂ (3×15 mL) and EtOAc (15 mL). The combined organic extracts were dried with MgSO₄, filtered and concentrated to give compound 66 (220 mg, 85% yield). m/z=492 (M+1)

Compound 67: A solution of 2-fluoroacetic acid (38 μL, 0.67 mmol) in CH₂Cl₂ (2 mL) was treated with N-(3-dimethylaminopropyl)-N′-ethylcarbodiimide hydrochloride (129 mg, 0.67 mmol) and DMAP (5 mg, 0.045 mmol) at room temperature under N₂. The mixture was stirred at room temperature for 15 min. Compound 66 (220 mg, 0.45 mmol) was then added and the mixture was stirred at room temperature for 24 h. The reaction mixture was concentrated, and the residue was purified by column chromatography (silica gel, eluting with 0 to 60% EtOAc in CH₂Cl₂) to give compound 67 (72 mg, 29% yield). m/z=552 (M+1).

T38: Compound 67 (70 mg, 0.13 mmol) and p-toluenesulfonic acid monohydrate (12 mg, 0.063 mmol) in toluene (7 mL) was heated at reflux while removing water by Dean-Stark apparatus for 5 h. The mixture was cooled to room temperature; diluted with EtOAc (10 mL); and washed with water (2×15 mL). The organic extract was dried with MgSO₄, filtered and concentrated. The residue was purified by column chromatography (silica gel, eluting with acetone in hexanes) to give compound T38 (33 mg, 49% yield) as a white solid. m/z=534 (M+1); ¹H NMR (400 MHz, CDCl₃) δ 8.00 (s, 1H), 6.02 (s, 1H), 5.51 (d, J=47.1, 2H), 3.19 (dt, J=13.4, 4.0 Hz, 1H), 3.02 (d, J=4.7 Hz, 1H), 2.46 (dq, J=13.3, 6.7 Hz, 1H), 2.23 (td, J=13.7, 4.2 Hz, 1H), 2.01-1.20 (m, 14H), 1.41 (s, 3H), 1.24 (d, J=6.8 Hz, 3H), 1.16 (s, 3H), 1.06 (s, 6H), 0.95 (s, 3H).

Compound 69: A mixture of compound 68 (898 mg, 1.94 mmol), NaOAc (286 mg, 3.49 mmol) and hydroxylamine hydrochloride (175 mg, 2.52 mmol) in EtOH (30 mL) and H₂O (2 mL) were stirred at room temperature for 16 h. The mixture was concentrated. The residue was partitioned between ethyl acetate (20 mL) and water (10 mL). The organic layer was separated, and the aqueous layer was extracted with ethyl acetate (2×20 mL). The combined organic extracts were washed with brine (20 mL), dried with Na₂SO₄, filtered, and concentrated to give compound 69 (863 mg, 93% yield) as a white solid, which was carried to the next step without further purification. m/z=479 (M+1).

Compound 70: Compound 69 (863 mg, 1.80 mmol) in MeCN (9 mL) was cooled to −10° C. 12 N aq. HCl (30 μL, 0.36 mmol), and a solution of N-chlorosuccinimide (241 mg, 1.80 mmol) in MeCN (9 mL) were added sequentially. The reaction was run at −10° C. for 30 min, and then ammonium hydroxide (28 wt. % aqueous, 3 mL, 21.5 mmol) was added. The reaction was stirred at room temperature for overnight. Ethyl acetate (30 mL) and water (20 mL) were added. The layers were separated. The aqueous layer was extracted with ethyl acetate (4×20 mL). The combined organic extracts were washed with brine (20 mL), dried with Na₂SO₄, filtered, and concentrated. The residue was purified by column chromatography (silica gel, 0-100% EtOAc in CH₂Cl₂) to give compound 70 (457 mg, 51% yield). m/z=494 (M+1).

Compound 71: A solution of 2-fluoroacetic acid (169 mg, 2.17 mmol) in CH₂Cl₂ (15 mL) was added to N-(3-dimethylaminopropyl)-N′-ethylcarbodiimide hydrochloride (EDC-HCl, 416 mg, 2.17 mmol) at room temperature under N₂. Catalytic amount of DMAP (8 mg, 0.072 mmol) was added. The mixture was stirred at room temperature for 15 min. Compound 70 (357 mg, 0.72 mmol) was then added. The mixture was stirred at room temperature for 2 h, and then concentrated. The residue was diluted with ethyl acetate (30 mL) and washed with water (2×20 mL). The combined aqueous washes were extracted with ethyl acetate (20 mL). The combined organic extracts were washed with brine (20 mL), dried with Na₂SO₄, filtered and concentrated. The residue was purified by column chromatography (silica gel, eluting with 0-100% EtOAc in hexanes) to give compound 71 (150 mg, 38% yield). m/z=554 (M+1).

Compound 72: Compound 71 (207 mg, 0.374 mmol) in 1,4-dioxane (3 mL) was heated at 100° C. for 5 h under N₂. The mixture was cooled to room temperature and concentrated. The residue was purified by column chromatography (silica gel, eluting with 0-30% EtOAc in hexanes) to give compound 72 (158 mg, 79% yield). m/z=536 (M+1).

Compound 73: Compound 72 (158 mg, 0.295 mmol) in MeOH (4 mL) was treated with NaOMe (25 wt. % in MeOH, 135 μL, 0.59 mmol) at room temperature. The reaction was heated at 55° C. for 2.5 h, and then was cooled to room temperature. 10% aq. NaH₂PO₄ (20 mL) was added.

The mixture was extracted with ethyl acetate (2×30 mL). The combined organic extracts were washed with brine (20 mL), dried with Na₂SO₄, filtered and concentrated. The residue was purified by column chromatography (silica gel, eluting with 0-40% EtOAc in hexanes) to give compound 73 (134 mg, 85% yield). m/z=536 (M+1).

T39: Compound 73 (134 mg, 0.25 mmol) was dissolved in DMF (3 mL) and cooled to 0° C. under N₂. A solution of 1,3-Dibromo-5,5-dimethylhydantoin (38 mg, 0.13 mmol) in DMF (1 mL) was added dropwise. The mixture was stirred at 0° C. for 2 h. Pyridine (61 μL, 0.75 mmol) was then added and the reaction was heated at 60° C. for 4 h. After cooled to room temperature, the mixture was diluted with EtOAc (20 mL) and washed with 1 N aq. HCl (10 mL), water (2×10 mL) and brine (10 mL). The organic extract was dried with MgSO₄, filtered, and concentrated. The residue was purified by column chromatography (silica gel, eluting with 0-50% EtOAc in hexanes) to give compound T39 (96 mg, 70% yield) as a white foam. m/z=534 (M+1); ¹H NMR (400 MHz, CDCl₃) δ 8.01 (s, 1H), 6.01 (s, 1H), 5.54 (d, J=46.3, 2H), 3.22 (dt, J=13.2, 3.9 Hz, 1H), 2.98 (d, J=4.7 Hz, 1H), 2.45 (dq, J=13.3, 6.7 Hz, 1H), 2.19 (td, J=13.8, 4.2 Hz, 1H), 2.01-1.20 (m, 14H), 1.41 (s, 3H), 1.24 (d, J=6.7 Hz, 3H), 1.14 (s, 3H), 1.06 (s, 3H), 1.05 (s, 3H), 0.94 (s, 3H).

Compound 74: Compound 5 (150 mg, 0.295 mmol) in 1,4-dioxane (3 mL) was cooled to 0° C. Triethylamine (124 μL, 0.886 mmol) and trifluoroacetic anhydride (45 μL, 0.325 mmol) were added sequentially. The reaction was stirred at room temperature for 19 h. The reaction mixture was combined with another same reaction starting with compound 5. The mixture was diluted with EtOAc (40 mL), and washed with sat. aq. NaHCO₃ (2×20 mL) and brine (10 mL). The organic extract was dried with MgSO₄, filtered and concentrated. The residue was purified by column chromatography (silica gel, eluting with 0-100% EtOAc in hexanes) to give compound 74 (130 mg, 56% yield) as a white solid. m/z=586 (M+1).

Compound 75: Compound 74 (125 mg, 0.213 mmol) in MeOH (2 mL) was treated with sodium methoxide (25 wt. % in MeOH, 100 μL, 0.44 mmol) at room temperature. The reaction was heated at 55° C. for 2 h, and then was cooled to room temperature. 10% aq. NaH₂PO₄ (20 mL) was added and the mixture was extracted with EtOAc (2×30 mL). The combined organic extracts were washed with brine (20 mL), dried with MgSO₄, filtered and concentrated. The residue was purified by column chromatography (silica gel, eluting with 0-40% EtOAc in hexanes) to give compound 75 (85 mg, 68% yield) as a white solid. m/z=586 (M+1).

T40: Compound 75 (82 mg, 0.14 mmol) was dissolved in DMF (0.7 mL) and cooled to 0° C. under N₂. A solution of 1,3-Dibromo-5,5-dimethylhydantoin (20 mg, 0.070 mmol) in DMF (0.2 mL) was added dropwise. The mixture was stirred at 0° C. for 1 h. DMF (0.5 mL) was added to dissolve the white solid precipitated during the reaction. The reaction was stirred at room temperature for 10 min. Pyridine (45 μL, 0.56 mmol) was then added and the reaction was heated at 55° C. for 5 h. The mixture was cooled to room temperature; diluted with EtOAc (20 mL); and washed with 1 N aq. HCl (10 mL), water (2×10 mL) and brine (10 mL). The organic extract was dried with MgSO₄, filtered, and concentrated. The residue was purified by column chromatography (silica gel, eluting with 0-40% acetone in hexanes) to give compound T40 (70 mg, 85% yield) as a white solid. m/z=584 (M+1); ¹H NMR (400 MHz, CDCl₃) δ 8.02 (s, 1H), 5.97 (s, 1H), 3.26-3.19 (m, 1H), 2.95 (d, J=4.7 Hz, 1H), 2.22 (td, J=13.8, 4.2 Hz, 1H), 1.96-1.83 (m, 2H), 1.80-1.20 (m, 12H), 1.45 (s, 3H), 1.25 (s, 3H), 1.16 (s, 3H), 1.14 (s, 3H), 1.06 (s, 3H), 1.05 (s, 3H), 0.95 (s, 3H).

Compound 76: To a solution of N-(3-dimethylaminopropyl)-N′-ethylcarbodiimide hydrochloride (170 mg, 0.886 mmol) in CH₂Cl₂ (3 mL) was added 2,2-difluoroacetic acid (85 mg, 0.886 mmol) in CH₂Cl₂ (3 mL) and DMAP (3.6 mg, 0.030 mmol) sequentially at room temperature. After stirring the reaction mixture for 15 min, a solution of compound 5 (150 mg, 0.295 mmol) in CH₂Cl₂ (4 mL) was added. The reaction was stirred at room temperature for another 19 h. The mixture was then diluted with CH₂Cl₂ (20 mL) and washed with water (15 mL). The aqueous wash was extracted with EtOAc (2×15 mL). The combined organic extracts were dried with Na₂SO₄, filtered and concentrated. The residue was purified by column chromatography (silica gel, eluting with 0-40% EtOAc in hexanes) to give compound 76 (106 mg, 63% yield) as a white solid. m/z=568 (M+1).

Compound 77: Compound 76 (106 mg, 0.187 mmol) in MeOH (1.8 mL) was treated with sodium methoxide (25 wt. % in MeOH, 85.5 μL, 0.373 mmol) at room temperature. The reaction was heated at 55° C. for 2 h, and then was cooled to room temperature. 10% aq. NaH₂PO₄ (20 mL) was added. The mixture was extracted with EtOAc (2×20 mL). The combined organic extracts were dried with MgSO₄, filtered and concentrated. The residue was purified by column chromatography (silica gel, eluting with 0-40% EtOAc in hexanes) to give compound 77 (90 mg, 85% yield) as a white solid. m/z=568 (M+1).

T41: Compound 77 (66 mg, 0.12 mmol) and 1,3-dibromo-5,5-dimethylhydantoin (17 mg, 0.058 mmol) were weighed in a round-bottom flask, and cooled to 0° C. DMF (1.2 mL) was added under N₂. The mixture was stirred at 0° C. for 1 h. Pyridine (38 μL, 0.47 mmol) was then added and the reaction was heated at 55° C. for 5 h. After cooled to room temperature, the mixture was diluted with EtOAc (20 mL) and washed with 1 N aq. HCl (10 mL), water (2×10 mL) and brine (10 mL). The organic extract was dried with MgSO₄, filtered, and concentrated. The residue was purified by column chromatography (silica gel, eluting with 0-40% EtOAc in hexanes) to give compound T41 (57 mg, 87% yield) as a white solid. m/z=566 (M+1); ¹H NMR (400 MHz, CDCl₃) δ 8.02 (s, 1H), 6.79 (t, J=52.2 Hz, 1H), 5.96 (s, 1H), 3.22 (dt, J=13.4, 4.4 Hz, 1H), 2.92 (d, J=4.7 Hz, 1H), 2.21 (td, J=13.8, 4.2 Hz, 1H), 1.96-1.83 (m, 2H), 1.80-1.20 (m, 12H), 1.45 (s, 3H), 1.25 (s, 3H), 1.15 (s, 3H), 1.13 (s, 3H), 1.05 (s, 6H), 0.95 (s, 3H).

Compound 79: Compound 35 (500 mg, 0.980 mmol) in CH₂Cl₂ (10 mL) was cooled to 0° C., and treated with triethylamine (0.55 mL, 3.95 mmol) and compound 78 (215 mg, 1.47 mmol). The reaction mixture was stirred at ambient temperature for 2 h, and then washed with water. The aqueous layer was separated and extracted with CH₂Cl₂. The combined organic extracts were dried with Na₂SO₄, filtered and concentrated. The residue was purified by column chromatography (silica gel, eluting with 60% EtOAc in hexanes) to give compound 79 (437 mg, 72% yield) as a white solid. m/z=620 (M+1).

Compound 80: A solution of compound 79 (437 mg, 0.705 mmol) in THF (15 mL) was treated with tetrabutylammonium hydroxide (1.0 M solution in methanol, 1.41 mL, 1.41 mmol) at room temperature. The reaction was stirred at room temperature for 11 h, and then was diluted with ethyl acetate. The mixture was washed with water and brine. The organic extract was dried with Na₂SO₄, filtered and concentrated. The residue was purified by column chromatography (silica gel, eluting with 30% EtOAc in hexanes) to give compound 80 (258 mg, 61% yield) as an off-white solid. m/z=602 (M+1).

T42: A solution of compound 80 (258 mg, 0.428 mmol) in CH₂Cl₂ (10 mL) was treated with trifluoroacetic acid (1 mL, 12.98 mmol) at room temperature. The reaction was stirred for 22 h, and then was concentrated. The residue was dissolved in toluene and concentrated. The residue was purified by column chromatography (silica gel, eluting with 30-100% EtOAc in hexanes) give compound T42 (178 mg, 76% yield) as a white solid. m/z=546 (M+1); ¹H NMR (400 MHz, CDCl₃) δ 8.02 (s, 1H), 5.97 (s, 1H), 4.77 (d, J=6.7 Hz, 2H), 3.33-3.20 (m, 1H), 3.04 (d, J=4.7 Hz, 1H), 2.27-2.16 (m, 2H), 2.00-1.20 (m, 14H), 1.45 (s, 3H), 1.25 (s, 3H), 1.15 (s, 3H), 1.11 (s, 3H), 1.05 (s, 6H), 1.00 (s, 3H).

Compound 81: A solution of triethylamine (0.266 mL, 1.90 mmol) and 2,2-difluoro-N-hydroxyethanimidamide (78.6 mg, 0.71 mmol) in CH₂Cl₂ (3.0 mL) was added dropwise to a solution of compound 35 (243 mg, 0.476 mmol) in CH₂Cl₂ (4.0 mL) at 0° C. under N₂. The mixture was stirred at room temperature for 18 h, and then partitioned between CH₂Cl₂ (40 mL) and water (40 mL). The aqueous phase was separated and extracted with CH₂Cl₂ (2×30 mL). The combined organic extracts were washed with brine, dried with Na₂SO₄, filtered and concentrated. The residue was purified by column chromatography (silica gel, eluting with 0-100% EtOAc in hexane) to give compound 81 (186 mg, 67% yield) as a solid. m/z=584.3 (M+1).

T43: To a solution of compound 81 (136.0 mg, 0.23 mmol) in anhydrous THF (10 mL) was added of tetrabutylammonium fluoride (1.0 M in THF, 0.70 mL, 0.70 mmol) at room temperature under N₂. The mixture was stirred at reflux for 5.5 h, and then was concentrated. The residue was partitioned between EtOAc (40 mL) and water (40 mL). The aqueous layer was separated and extracted with EtOAc (3×30 mL). The combined organic extracts were washed with brine, dried Na₂SO₄, filtered and concentrated. The residue was purified by column chromatography (silica gel, eluting with 0-50% acetone in CH₂Cl₂) to give T43 (73 mg, 55% yield) as a solid. m/z=566.4 (M+1); ¹H NMR (400 MHz, CDCl₃) δ 8.02 (s, 1H), 6.75 (t, J=52.5 Hz, 1H), 5.97 (s, 1H), 3.29-3.23 (m, 1H), 3.00 (d, J=4.7 Hz, 1H), 2.25 (td, J=13.9, 4.2 Hz, 1H), 2.01-1.88 (m, 2H), 1.51-1.20 (m, 12H), 1.45 (s, 3H), 1.25 (s, 3H), 1.15 (s, 3H), 1.10 (s, 3H), 1.06 (s, 3H), 1.05 (s, 3H), 0.96 (s, 3H).

Compound 82: Difluoroacetic anhydride (52 μL, 0.45 mmol) was added to a solution of compound 36 (0.19 g, 0.38 mmol) and pyridine (46 μL, 0.56 mmol) in CH₂Cl₂ (4 mL) at 0° C. under N₂. The reaction was stirred at 0° C. for 30 min, and then at room temperature for 75 min. The reaction mixture was diluted with EtOAc (25 mL) and washed with 1 N aq. HCl (20 mL), water (20 mL) and brine (10 mL). The organic extract was dried with Na₂SO₄, filtered and concentrated. The residue was purified by column chromatography (silica gel, eluting with 0-40% EtOAc in CH₂Cl₂) to give compound 82 (95 mg, 43% yield) and a mixture of compound 82 and T44 (4/1, 80 mg, 36% yield). Compound 82: m/z=584.3 (M+1).

T44: p-Toluenesulfonic acid monohydrate (13 mg, 0.068 mmol) was added to a mixture of compound 82 (80.0 mg, 0.14 mmol) in toluene (8 mL) at room temperature. The reaction was heated at reflux while removing water by Dean-Stark apparatus for 3 h. After cooled to room temperature, the mixture was partitioned between EtOAc (30 mL) and water (10 mL). The organic extract was washed with water (2×10 mL) and brine (10 mL); dried with Na₂SO₄; filtered and concentrated. The residue was purified by column chromatography (silica gel, eluting with EtOAc in hexanes) to give compound T44 (24 mg, 31% yield) as a white solid. m/z=566.4 (M+1); ¹H NMR (400 MHz, CDCl₃) δ 8.02 (s, 1H), 6.83 (t, J=51.8 Hz, 1H), 5.97 (s, 1H), 3.25-3.17 (m, 1H), 3.05 (d, J=4.7 Hz, 1H), 2.25 (td, J=13.9, 4.2 Hz, 1H), 2.01-1.88 (m, 2H), 1.51-1.20 (m, 12H), 1.45 (s, 3H), 1.25 (s, 3H), 1.15 (s, 6H), 1.06 (s, 6H), 0.96 (s, 3H).

Compound 83: Trifluoroacetic anhydride (74 μL, 0.52 mmol) was added to a solution of compound 36 (0.22 g, 0.44 mmol) and pyridine (53 μL, 0.65 mmol) in CH₂Cl₂ (5 mL) at room temperature. The mixture was stirred at 40° C. for 75 min. Additional amount of trifluoroacetic anhydride (20 μL, 0.14 mmol) was added and the resultant mixture was stirred for additional 1 h. Compound 83 was completely consumed. The reaction mixture was then cooled to room temperature; diluted with EtOAc (25 mL); and washed with 1 N aq. HCl (20 mL), water (2×20 mL) and brine (10 mL). The organic extract was dried with Na₂SO₄, filtered and concentrated. The residue was purified by column chromatography (silica gel, eluting with 0-75% EtOAc in hexanes) to give compound 83 (0.22 g, 84% yield) as a white solid.

T45: Burgess reagent (0.42 g, 1.76 mmol) was added to a solution of compound 83 (0.21 g, 0.35 mmol) in THF (4 mL) at room temperature. The reaction was stirred at 70° C. for 7 h. Additional amount of Burgess reagent (210 mg, 0.88 mmol) was added and the resultant mixture was stirred at 70° C. for overnight. Compound 83 was completely consumed. The reaction mixture was cooled to room temperature, and then partitioned between EtOAc (25 mL) and water (10 mL). The aqueous phase was separated and extracted with EtOAc (20 mL). The combined organic extracts were washed with water (2×10 mL) and brine (10 mL); dried with Na₂SO₄; filtered and concentrated. The residue was purified by column chromatography (silica gel, eluting with 0-40% EtOAc in hexanes) to give compound T45 (0.11 g, 54% yield) as a white solid. m/z=584.3 (M+1); ¹H NMR (400 MHz, CDCl₃) δ 8.02 (s, 1H), 5.98 (s, 1H), 3.24-3.16 (m, 1H), 3.05 (d, J=4.7 Hz, 1H), 2.26 (td, J=14.0, 4.2 Hz, 1H), 2.01-1.88 (m, 2H), 1.80-1.20 (m, 12H), 1.46 (s, 3H), 1.25 (s, 3H), 1.16 (s, 6H), 1.06 (s, 3H), 1.05 (s, 3H), 0.96 (s, 3H).

Compound 84: Propionic anhydride (0.076 mL, 0.59 mmol) was added to a solution of compound 36 (0.25 g, 0.49 mmol) and pyridine (60 μL, 0.74 mmol) in CH₂Cl₂ (5 mL). The reaction was stirred at 40° C. for 2 h. The mixture was diluted with EtOAc (25 mL) and washed with 1 N aq. (1 N, 20 mL), water (2×20 mL) and brine (10 mL). The organic extract was dried with Na₂SO₄, filtered and concentrated. The residue was purified by column chromatography (silica gel, eluting with 0-100% EtOAc in hexanes) to give compound 84 (0.21 g, 76% yield) as a white solid. m/z=562.3 (M+1).

T46: Burgess reagent (0.21 g, 0.88 mmol) was added to a mixture of compound 84 (99 mg, 0.18 mmol) in THF (2 mL) at room temperature. The reaction was stirred at 70° C. for overnight, and then cooled to room temperature. The mixture was partitioned between EtOAc (25 mL) and water (10 mL). The aqueous phase was separated and extracted with EtOAc (20 mL). The combined organic extracts were washed with water (2×10 mL) and brine (10 mL), dried with Na₂SO₄, filtered and concentrated. The residue was purified by column chromatography (silica gel, eluting with 0-80% EtOAc in hexanes) to give compound T46 (71 mg, 74% yield) as a white solid. m/z 544.3 (M+1); ¹H NMR (400 MHz, CDCl₃) δ 8.02 (s, 1H), 5.95 (s, 1H), 3.13 (dt, J=13.4, 4.1 Hz, 1H), 2.96 (d, J=4.7 Hz, 1H), 2.85 (q, J=7.6 Hz, 2H), 2.21-2.13 (m, 1H), 2.01-1.20 (m, 14H), 1.44 (s, 3H), 1.35 (t, J=7.5 Hz, 3H), 1.24 (s, 3H), 1.17 (s, 3H), 1.15 (s, 3H), 1.05 (s, 3H), 1.04 (s, 3H), 0.94 (s, 3H).

Compound 87: To a solution of compound 85 (6.58 g, 14.0 mmol) in CH₂Cl₂ (66 mL) was added oxalyl chloride (3.69 mL, 42.1 mmol) and DMF (0.11 mL, 1.40 mmol) sequentially at 0° C. under N₂. The mixture was stirred at room temperature for 2 h, and then concentrated. The residue was dissolved in toluene (3×60 mL) and concentrated to remove residual oxalyl chloride. Compound 86 was obtained as a yellow solid, which was used in the next step without further purification.

Compound 86 was dissolved in CH₂Cl₂ (100 mL) and cooled to 0° C. Triethylamine (7.83 mL, 56.2 mmol) and 2-fluoro-N-hydroxyacetimidamide (1.94 g, 21.1 mmol) were added sequentially. The mixture was stirred at room temperature for 4 h, and then washed with water (20 mL). The aqueous phase was separated, and extracted with CH₂Cl₂ (20 mL). The combined organic extracts were dried with Na₂SO₄, filtered and concentrated. The residue was purified by column chromatography (silica gel, eluting with 0-100% EtOAc in hexanes) to give compound 87 (6.81 g, 89% yield) as a yellow solid. m/z=543.3 (M+1).

Compound 88: Compound 87 (4.782 g, 8.811 mmol) was dissolved in anhydrous o-xylene (48 mL). Triethylamine (6.75 mL, 48.5 mmol) and propylphosphonic anhydride (50 wt. % solution in EtOAc, 17.3 mL, 29.1 mmol) were added sequentially. The mixture was heated at reflux for 7 h, and then was cooled to 0° C. Sat. aq. NaHCO₃ (100 mL) was added slowly. After the addition was complete, the mixture was extracted with CH₂Cl₂ (100 mL). The organic extract was washed with sat. aq. NaHCO₃ (100 mL) and water (100 mL). The combined aqueous washes were extracted with EtOAc (2×150 mL). The organic extracts were dried with Na₂SO₄; filtered through a pad of silica gel (25 g); and eluted with EtOAc (100 mL). The filtrate was concentrated. The residue was purified by column chromatography (silica gel, eluting with 0-100% EtOAc in hexanes) to give compound (1.65 g, 36% yield) as an off-white solid. m/z=525.3 (M+1).

Compound 89: A mixture of compound 88 (2.865 g, 5.460 mmol) in ethyl formate (13.2 mL, 164 mmol) was cooled to 0° C. under N₂. Sodium methoxide (25 wt. % in MeOH, 12.3 mL, 53.8 mmol) was added. The mixture was stirred at room temperature for 1.5 h, and then cooled to 0° C. HCl (6 M aqueous, 9.10 mL, 54.6 mmol), EtOH (55 mL) and hydroxylamine hydrochloride (569 mg, 8.19 mmol) were added sequentially. The mixture was heated at 60° C. for 3 h; cooled to room temperature; and concentrated. The residue was dissolved in EtOAc (60 mL) and washed with water (2×30 mL). The combined aqueous washes were extracted with EtOAc (2×20 mL). The combined organic extracts were dried with MgSO₄, filtered and concentrated. The residue was purified by column chromatography (silica gel, eluting with 0-50% EtOAc in hexanes) to give compound 89 (2.754 g, 92% yield) as a white solid. m/z=550.3 (M+1).

Compound 90: Compound 89 (2.754 g, 5.010 mmol) in MeOH (50 mL) was treated with sodium methoxide (25 wt. % in MeOH, 2.29 mL, 10.0 mmol) at room temperature under N₂. The mixture was heated at 55° C. for 1.5 h; cooled to 0° C.; and treated with 10% aq. NaH₂PO₄ (30 mL). The mixture was partitioned between EtOAc (50 mL) and brine (30 mL). The aqueous phase was separated and extracted with EtOAc (50 mL). The combined organic extracts were dried with MgSO₄, filtered and concentrated. The residue was column chromatography (silica gel, eluting with 0-30% acetone in hexanes) to give compound 90 (2.52 g, 92% yield) as a white solid. m/z=550.3 (M+1).

T12: Compound 90 (2.570 g, 4.675 mmol) was dissolved in DMF (12 mL) and cooled to 0° C. under N₂. 1,3-Dibromo-5,5-dimethylhydantoin (735 mg, 2.57 mmol) was added, followed by additional amount of DMF (11 mL). The mixture was stirred at 0° C. for 2 h. Pyridine (1.51 mL, 18.7 mmol) was added. The mixture was heated at 60° C. for 4 h, and then was cooled to room temperature. The mixture was diluted with EtOAc (50 mL), and washed sequentially with 1 N aq. HCl (30 mL), water (2×30 mL), and brine (20 mL). The aqueous washes were combined and extracted with EtOAc (2×30 mL). The combined organic extracts were washed with water (2×30 mL) and brine (20 mL); dried with Na₂SO₄, filtered and concentrated. The residue was purified by column chromatography (silica gel, eluting with 0-30% acetone in hexanes) to give compound T12 (2.456 g, 96% yield) as a light yellow solid. m/z=548.3 (M+1); ¹H NMR (400 MHz, CDCl₃) δ 8.02 (s, 1H), 5.97 (s, 1H), 5.44 (d, J=46.5 Hz, 2H), 3.26 (m, 1H), 3.03 (d, J=4.7 Hz, 1H), 2.24 (td, J=13.8, 4.1 Hz, 1H), 1.87-2.01 (m, 2H), 1.45 (s, 3H), 1.25 (s, 3H), 1.20-1.80 (m, 12H), 1.15 (s, 3H), 1.10 (s, 3H), 1.06 (s, 3H), 1.05 (s, 3H), 0.95 (s, 3H).

### Example 2: Nitric Oxide Suppression Data

Tissue Culture. RAW 264.7, a mouse macrophage cell line, was obtained from American Type Culture Collection (Manassas VA) and maintained in the log phase of growth in Roswell Park Memorial Institute Medium 1640 (RPMI 1640) supplemented with 10% heat inactivated fetal bovine serum (FBS) and 1% penicillin-streptomycin. Cells were cultured and maintained in a humidified incubator at 37° C. under 5% C02. Cells were sub-cultured every 3 days. Alpha Mouse Liver (AML-12) cells were purchased from ATCC and cultured in DMEM/F12 medium supplemented with 10% FBS, 1% penicillin/streptomycin. All cell culture supplies were obtained from Life Technologies (Grand Island, NY) and VWR (Radnor, PA).

Nitric Oxide Suppression Assay. RAW 264.7 cells were plated 1 day in advance of experimental treatments at a concentration of 30,000 cells per well onto Falcon-96 well clear bottom plates (Corning, NY) in a total volume of 200 μL per well using RPMI 1640 supplemented with 0.5% fetal bovine serum and 1% penicillin-streptomycin. The next day, cells were pretreated with compounds serially diluted from 1000× stocks. All compounds were dissolved in dimethyl sulfoxide (DMSO) at 10 mM stock solutions. Compounds were subsequently diluted in DMSO and RPMI 1640. Each well received a final concentration of 0.1% DMSO. Cells were pretreated for 2 hours and incubation at 37° C., followed by treatment with 20 ng/mL of interferon gamma (R&D Systems, Minneapolis, MN) per well for 24 hours. The next day, a nitrite standard was serially diluted from 100 μM to 1.6 μM in RPMI 1640. Afterwards, 50 μL of cell culture supernatant was transferred from each well into a new Falcon-96 well clear bottom plate. Nitrite was measured as surrogate for nitric oxide using Promega's Griess Detection Kit #G2930 (Madison, WI) which involves the addition of 50 μL of the provided sulfanilamide solution to each well of the transferred cell culture supernatant and standards, followed by a 10-minute incubation at room temperature. Next, 50 μL of the provided N−1-napthylethylenediamine dihydrochloride (NED) solution was added to the sulfanilamide reaction and incubated for 10 minutes at room temperature in the dark. Afterwards, air bubbles were removed using ethanol vapor and absorbance was measured using a Spectramax M2e plate reader with a wavelength set to 525 nm. Viability was assessed using WST-1 cell proliferation reagent from Roche (Basel, Switzerland). After media was removed for the Nitric Oxide suppression assay, 15 μL of WST-1 reagent was added to each well of cells. Plates were briefly mixed on an orbital shaker and the cells were incubated at 37° C. for 30 minutes. Absorbance was measured using a Spectramax M2e plate reader with wavelengths set to 440 nm and 700 nm.

For the ability of compounds to suppress the increase in nitric oxide release caused by interferon gamma, the absolute amount of nitrite that was produced in each well was extrapolated from the nitrite standards using a linear regression fit. All values were then normalized to the DMSO-interferon gamma treated wells and plotted as percent nitric oxide. IC₅₀ values were calculated with a dependence on WST1 viability using Excel and/or GraphPad Prism (San Diego, CA). The data is shown in Table 2.

TABLE 2
Nitric Oxide Inhibition (NO IC₅₀) and NO IC₅₀ Relative to RTA 402.

| | | | NO IC₅₀ | |
|---|---|---|---|---|
| | | NO IC₅₀ | Relative to | |
| | | (nM) | RTA 402 | |
| | | (Mean ± | (fold)^{a} | # of |
| ID # | Structure | SD) | (Mean ± SD) | Repeats |
| CC2 | 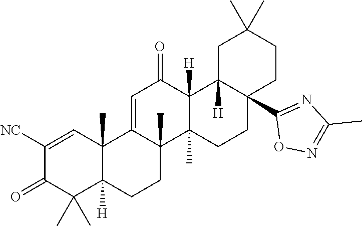 | 1.98 ± 0.56 | 0.70 ± 0.18 | 13 |
| T1 | 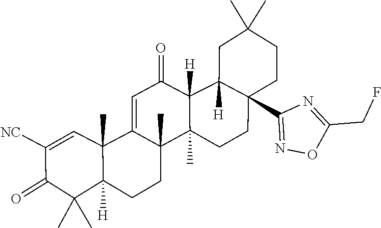 | 1.24 ± 0.38 | 0.52 ± 0.12 | 8 |
| T41 | 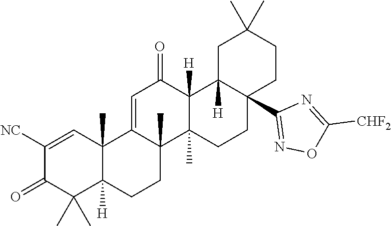 | 9.84 ± 3.52 | 2.79 ± 0.64 | 4 |
| T40 | 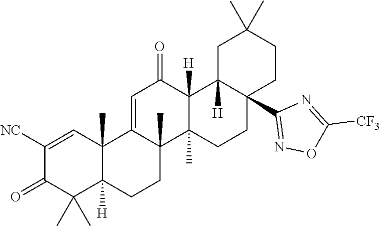 | 24.25 ± 7.64 | 7.76 ± 2.19 | 5 |
| T24 | 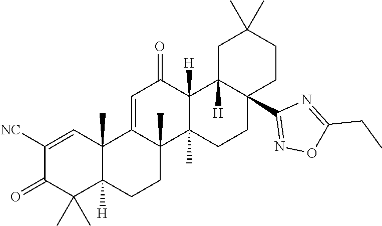 | 9.21 ± 2.66 | 2.56 ± 0.62 | 6 |
| T2 | 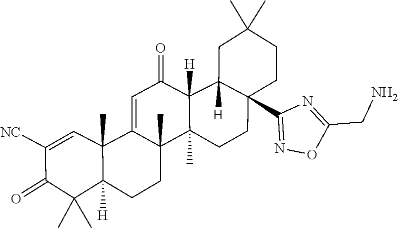 | 1.15 ± 0.44 | 0.38 ± 0.09 | 10 |
| T12 | 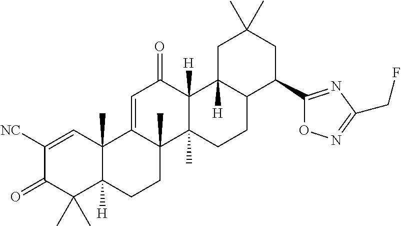 | 1.27 ± 0.34 | 0.52 ± 0.12 | 8 |
| T43 | 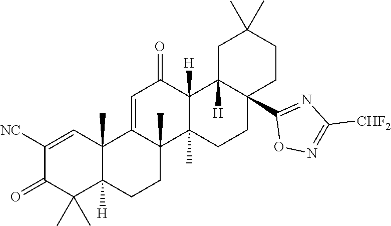 | 6.45 ± 1.29 | 1.90 ± 0.49 | 4 |
| T22 | 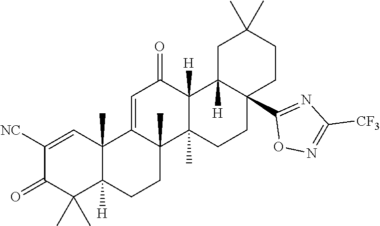 | 23.95 ± 10.04 | 7.71 ± 2.33 | 5 |
| T23 | 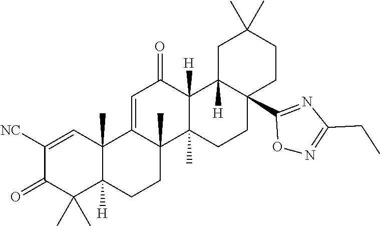 | 4.85 ± 2.61 | 1.93 ± 0.54 | 7 |
| CC1 | 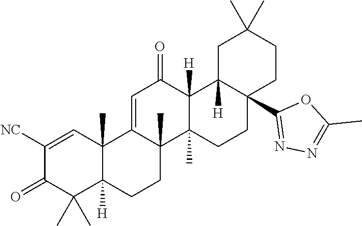 | 2.05 ± 1.36 | 0.78 ± 0.10 | 10 |
| T11 | 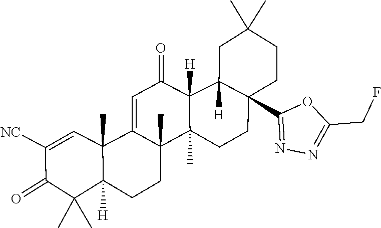 | 0.98 ± 0.54 | 0.37 ± 0.07 | 10 |
| T44 | 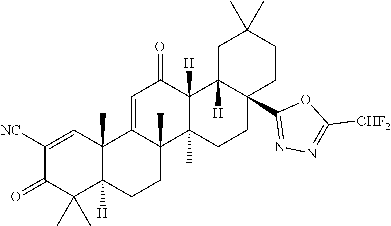 | 4.24 ± 2.23 | 1.24 ± 0.04 | 4 |
| T45 | 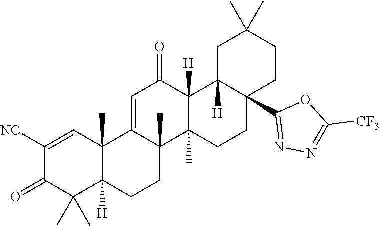 | 12.90 ± 6.38 | 3.81 ± 0.34 | 4 |
| T46 | 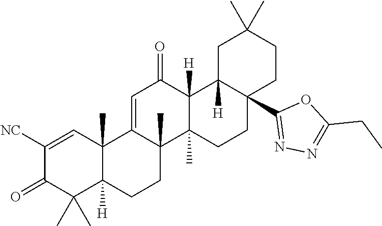 | 5.44 ± 2.81 | 1.60 ± 0.29 | 4 |
| CC3 | 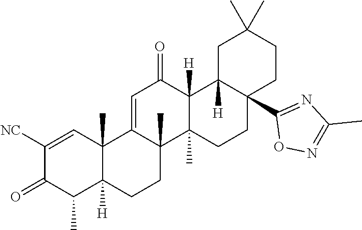 | 1.34 ± 0.52 | 0.40 ± 0.06 | 11 |
| T34 | 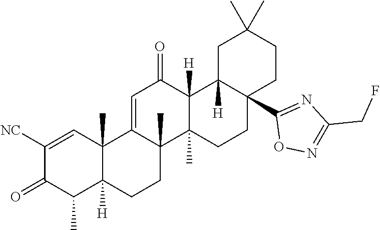 | 0.93 ± 0.52 | 0.28 ± 0.06 | 9 |
| T35 | 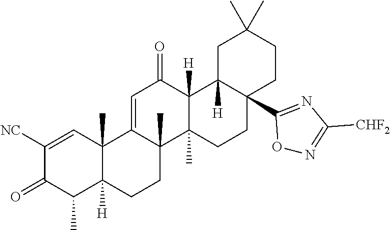 | 5.48 ± 3.04 | 1.21 ± 0.21 | 4 |
| T36 | 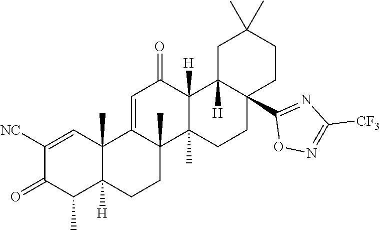 | 17.79 ± 9.89 | 3.95 ± 0.84 | 4 |
| T37 | 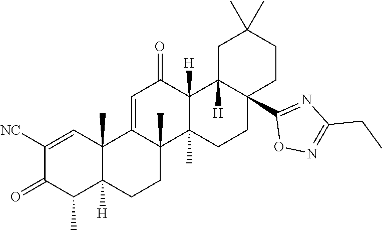 | 3.45 ± 1.87 | 0.76 ± 0.12 | 4 |
| CC4 | 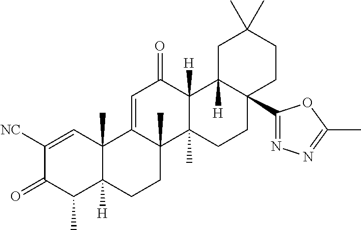 | 0.66 ± 0.06 | 0.51 ± 0.02 | 3 |
| T38 | 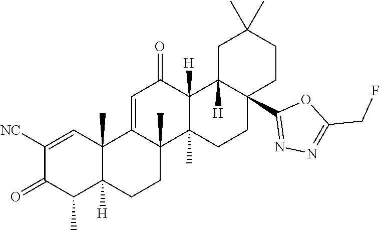 | 0.46 ± 0.02 | 0.20 ± 0.02 | 2 |
| T3 | 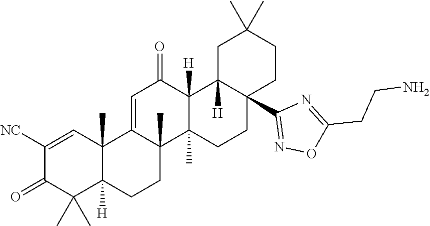 | 2.60 ± 0.03 | 0.69 ± 0.22 | 2 |
| T4 | 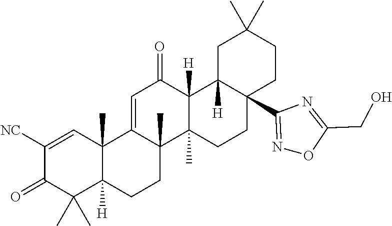 | 1.82 ± 0.13 | 0.48 ± 0.13 | 2 |
| T5 | 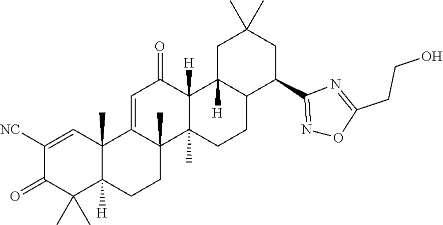 | 1.57 ± 0.05 | 0.42 ± 0.15 | 2 |
| T6 | 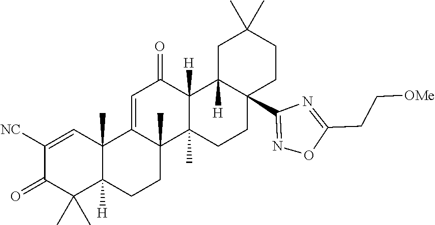 | 2.95 ± 0.11 | 0.79 ± 0.29 | 2 |
| T7 | 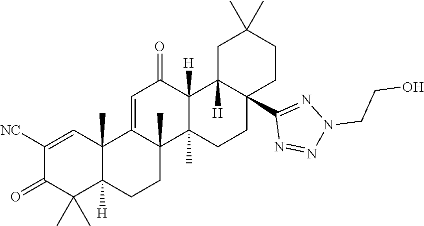 | 1.13 ± 0.18 | 0.43 ± 0.13 | 7 |
| T8 | 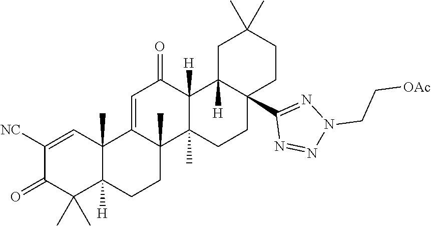 | 0.64 | 0.41 | 1 |
| T9 | 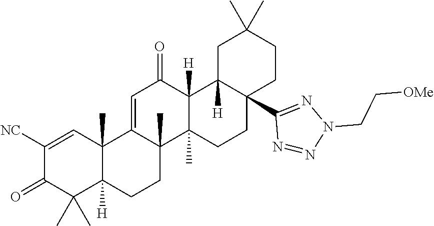 | 1.27 | 0.82 | 1 |
| T10 | 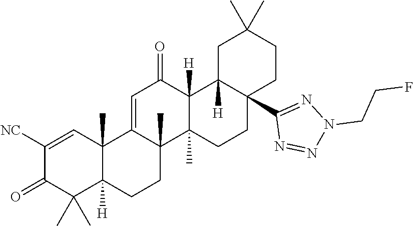 | 2.20 ± 0.20 | 1.62 ± 0.16 | 3 |
| T13 | 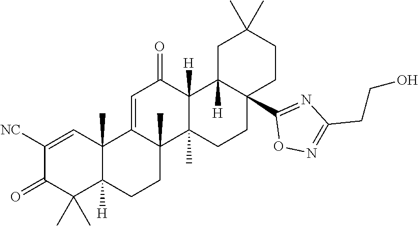 | 0.56 ± 0.26 | 0.22 ± 0.07 | 5 |
| T14 | 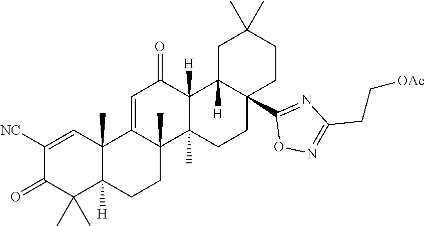 | 0.48 | 0.31 | 1 |
| T15 | 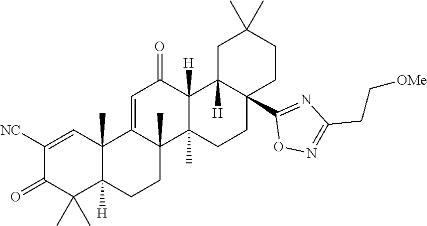 | 0.60 ± 0.05 | 0.38 ± 0.04 | 2 |
| T16 | 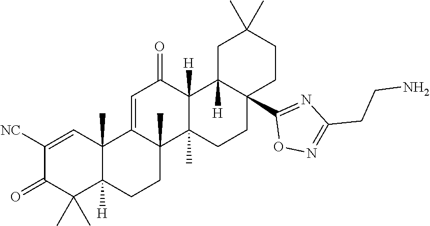 | 1.26 ± 0.12 | 0.94 ± 0.19 | 3 |
| T17 | 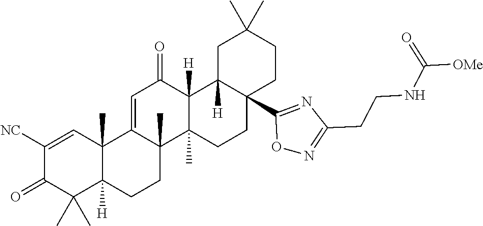 | 0.75 | 0.48 | 1 |
| T18 | 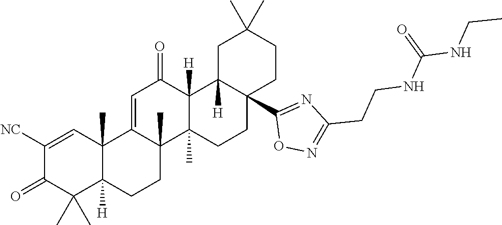 | 1.33 | 0.86 | 1 |
| T19 | 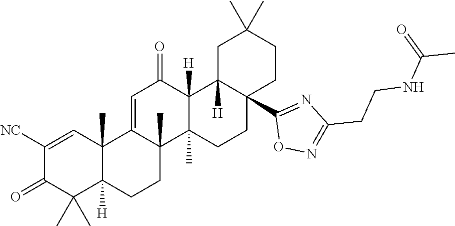 | 0.94 | 0.60 | 1 |
| T20 | 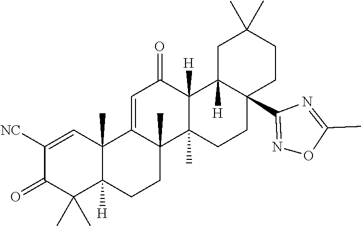 | 3.79 ± 1.58 | 1.30 ± 0.26 | 6 |
| T21 | 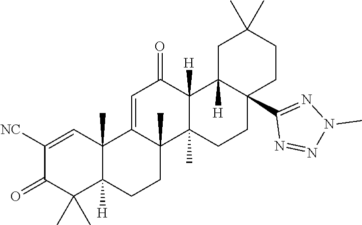 | 1.89 ± 0.67 | 0.76 ± 0.36 | 8 |
| T25 | 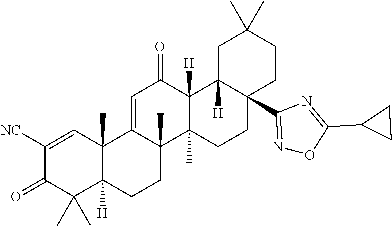 | 11.22 ± 2.09 | 3.08 ± 1.54 | 2 |
| T26 | 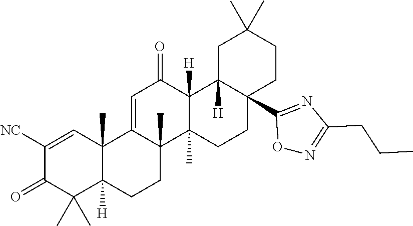 | 7.82 | 4.83 | 1 |
| T27 | 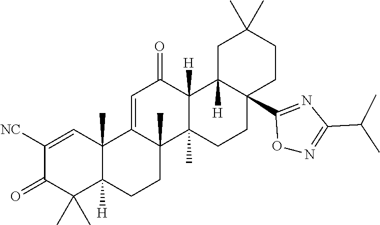 | 6.03 | 3.72 | 1 |
| T28 | 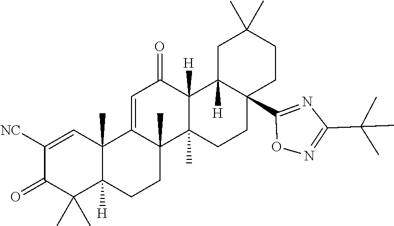 | 22.27 | 13.75 | 1 |
| T29 | 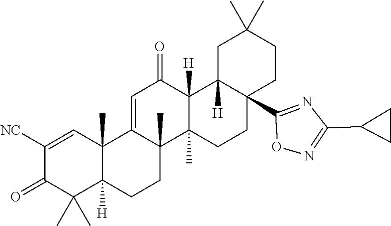 | 4.03 | 2.49 | 1 |
| T30 | 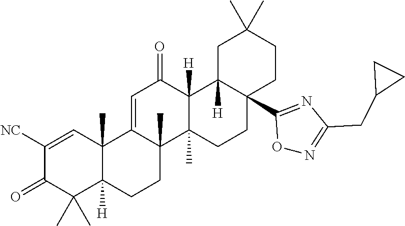 | 4.82 | 2.97 | 1 |
| T31 | 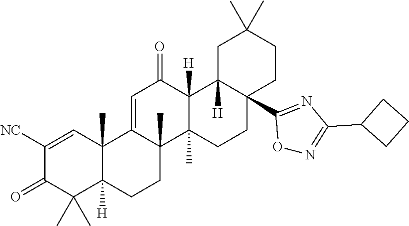 | 9.34 | 5.76 | 1 |
| T32 | 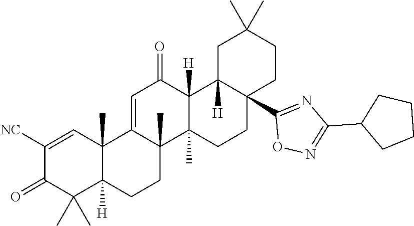 | 20.80 | 12.84 | 1 |
| T33 | 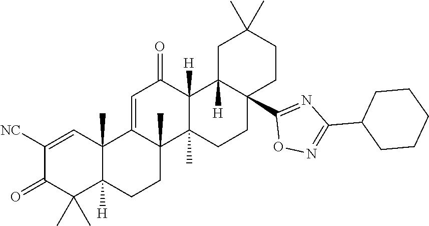 | 25.62 | 15.81 | 1 |
| T39 | 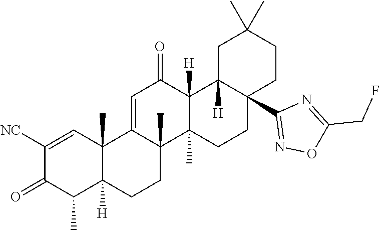 | 1.98 | 0.42 | 1 |
| T42 | 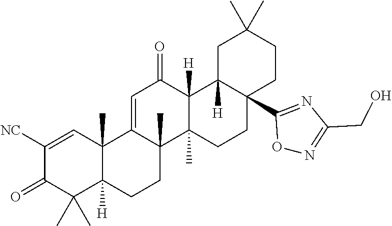 | 0.44 | 0.32 | 1 |

^{a}Average of ratios from replicate experiments.

TABLE 3
NO IC₅₀ Relative to Comparison Compounds.

| | | | NO IC₅₀ Relative | |
|---|---|---|---|---|
| | | | to Comparison | |
| | | | Compound in | |
| | | Comparison | Same | # of |
| ID # | Structure | Compound | Experiment^{b} | Repeats |
| CC2 | 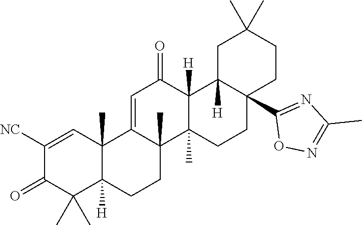 | CC2 | 1.00 | N/A |
| T1 | 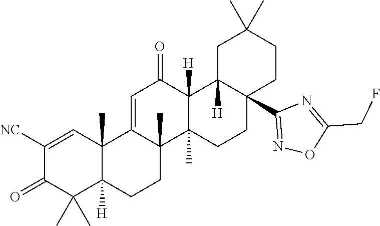 | CC2 | 0.67 ± 0.10 | 5 |
| T2 | 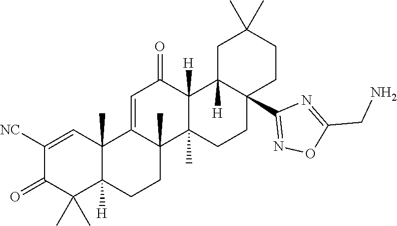 | CC2 | 0.69 ± 0.04 | 4 |
| T12 | 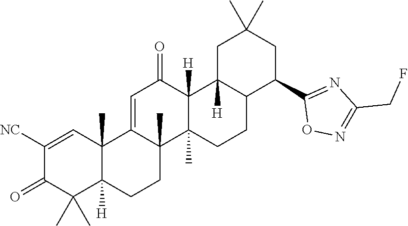 | CC2 | 0.65 ± 0.04 | 5 |
| T13 | 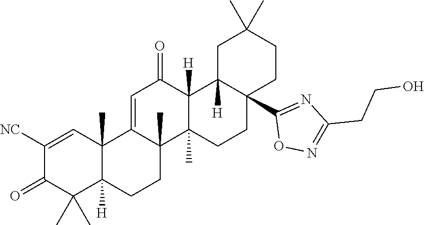 | CC2 | 0.30 ± 0.07 | 4 |
| T20 | 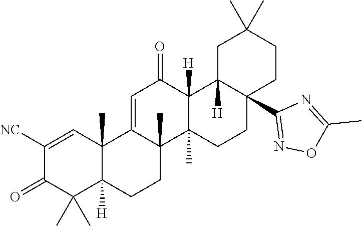 | CC2 | 1.85 ± 0.49 | 6 |
| CC1 | 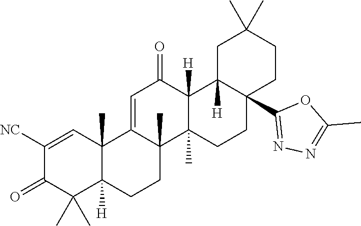 | CC1 | 1.00 | N/A |
| T11 | 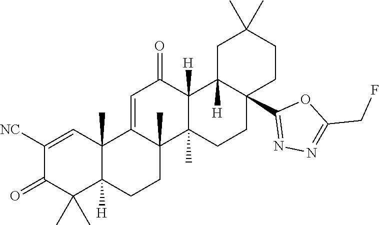 | CC1 | 0.40 ± 0.09 | 4 |
| T44 | 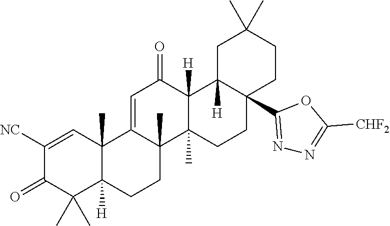 | CC1 | 1.55 ± 0.16 | 4 |
| CC3 | 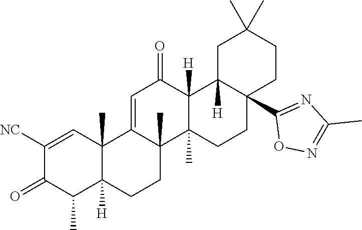 | CC3 | 1.00 | N/A |
| T34 | 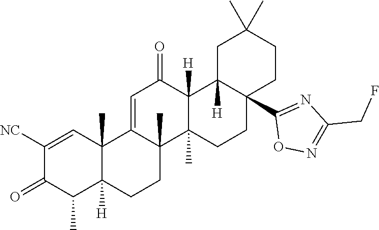 | CC3 | 0.70 ± 0.11 | 4 |

^{b}Average of ratios from experiments with direct comparisons.

### Example 3: CYP3A4 Inhibition

Methods. Several compounds were evaluated for CYP3A4 (midazolam) inhibition in human liver microsomes at 1 μM. CYP3A4 inhibition was tested using an in vitro assay as generally described in Dierks et al. (Drug Metabolism Deposition, 29:23-29, 2001, which is incorporated by reference herein). Each sample, containing 0.1 mg/mL human liver microsomes, 5 μM midazolam as substrate, and 1 μM of test compound, was incubated at 37° C. for 10 min. Following incubation, the metabolite 1-hydroxymidazolam was measured using HPLC-MS/MS. Peak areas corresponding to the metabolite of the substrate were recorded. The percent of control activity was then calculated by comparing the peak area obtained in the presence of the test compound to that obtained in the absence of the test compound. Subsequently, the percent inhibition was calculated by subtracting the percent control activity from 100 for each compound. The results of the CYP3A4 assays are show below in Tables 4-7.

TABLE 4
CYP3A4 (Midazolam) Inhibition.

| Compound ID | Structure | % Inhibition |
|---|---|---|
| RTA 402 | 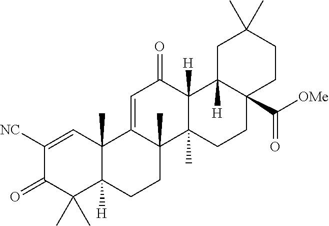 | 48.4 |
| RTA 408 | 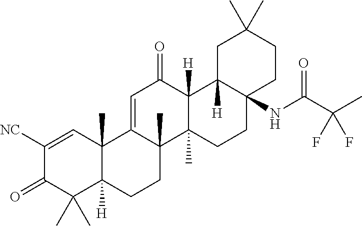 | 56.6 |
| T1 | 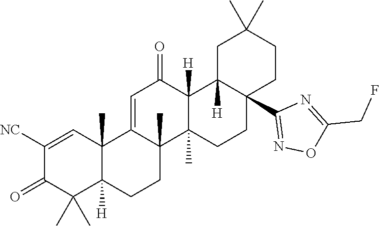 | 29.1 |
| T2 | 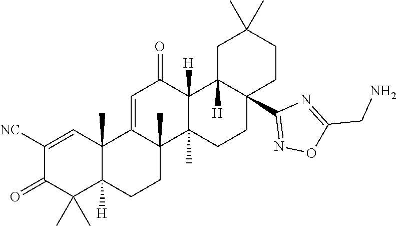 | 21.4 |
| T11 | 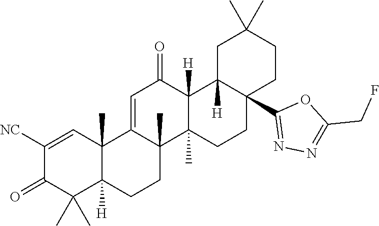 | 17.7 |
| T12 | 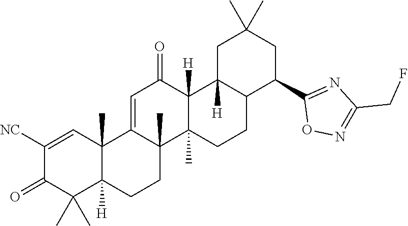 | 29.7 |
| T34 | 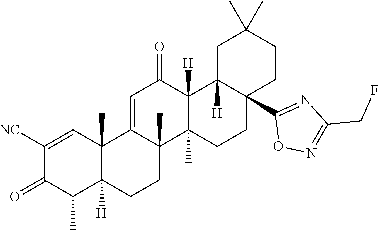 | 29.4 |

Comparison compound CC1 and T11, both comprising a 1,3,4-oxadiazole-2,5-diyl moiety, were also tested for CYP3A4 inhibition. The results of the CYP3A4 assay are shown below in Table 5.

TABLE 5
CYP3A4 (Midazolam) Inhibition of T11 Compared with CC1.

| Compound ID | Structure | % Inhibition |
|---|---|---|
| CC1 | 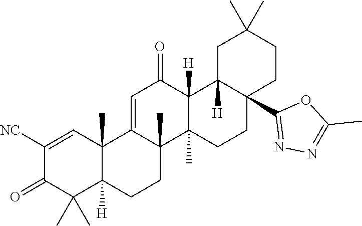 | 20.7 |
| T11 | 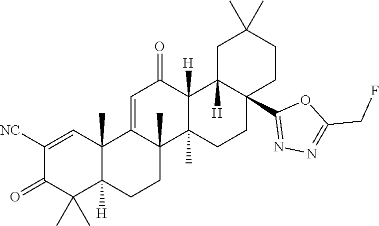 | 17.7 |

Comparison compound CC2 and T12, both comprising a 1,2,4 oxadiazole-3,5-diyl moiety, were also tested for CYP3A4 inhibition. The results of the CYP3A4 assay are shown below in Table 6.

TABLE 6
CYP3A4 (Midazolam) Inhibition of T12 Compared with CC2.

| Compound ID | Structure | % Inhibition |
|---|---|---|
| CC2 | 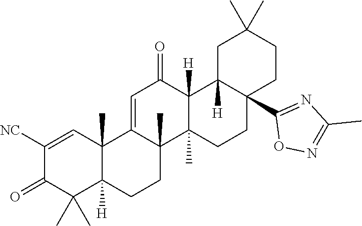 | 45.8 |
| T12 | 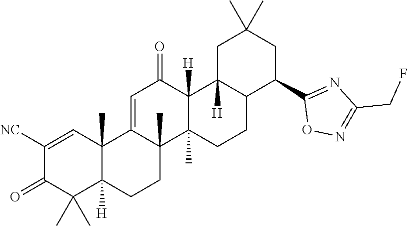 | 29.7 |

Comparison compound CC3 and T34, both comprising a 1,2,4 oxadiazole-3,5-diyl moiety and monomethyl substitution at C4, were also tested for CYP3A4 inhibition. The results are shown below in Table 7.

TABLE 7
CYP3A4 (Midazolam) Inhibition of T34 Compared with CC3.

| Compound ID | Structure | % Inhibition |
|---|---|---|
| CC3 | 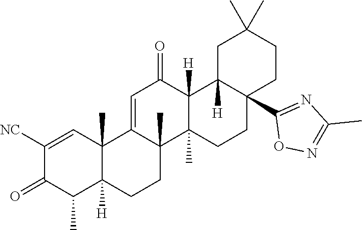 | 37.7 |
| T34 | 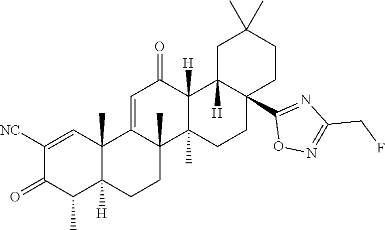 | 29.4 |

### Example 4: Glutathione Assay

The effect of compound treatment on total glutathione levels was evaluated in the mouse AML-12 hepatocyte cell line. Glutathione—a tripeptide consisting of cysteine, glutamate, and glycine—is the major thiol-containing protein within the cell and regulates cellular redox balance. Glutathione also plays important roles in detoxification, protein glutathionylation, and iron-sulfur cluster biogenesis (Bachhawat and Yadav, 2018). Nrf2 regulates the expression of many genes involved in glutathione synthesis and metabolism, including both subunits of glutamate cysteine ligase (GCL), the enzyme that catalyzes the rate limiting step of glutathione biosynthesis (Thimmulappa et al., 2002).

AML-12 cells were plated in white, clear-bottom 96-well plates at a density of 8,000 cells/well in 200 μL of DMEM/F12 medium supplemented with 10% FBS and 1% penicillin/streptomycin. The next day, cells were treated with vehicle (DMSO) or test compound (0.03 nM to 1000 nM). Each well received a final concentration of 0.1% DMSO. Cells were incubated at 37° C., 5% C02 for 24 hours. Total glutathione concentrations were measured using the GSH-Glo Glutathione Assay kit (Promega) according to the manufacturer's instructions. Briefly, a standard curve was prepared by serially diluting the provided glutathione solution. Final concentrations of total glutathione standards were 5, 2.5, 1.25, 0.625, 0.313, 0.156, 0.078, 0.039, and 0.0195 M. Following media removal from sample wells, 100 μL of glutathione reaction mix consisting of GSH-Glo reaction buffer, Glutathione S-Transferase, Luciferin-NT, and TCEP was added to each sample well and to all standard curve wells. After a 30-minute incubation at room temperature, 100 μL of Luciferin detection reagent was added to all sample and standard wells and incubated for 15 minutes. Luminescence was measured using a PHERAstar plate reader. The EC₅₀ value was determined using Excel and GraphPad Prism software. The basal concentration of glutathione was set to 0% and the highest concentration of glutathione produced following treatment with a test compound was set to 100% and a dose-response curve was generated. The dose-response curve was fit using nonlinear regression analysis and used to extrapolate the EC₅₀ value. The EC₅₀ value is defined as the concentration of test compound required to increase glutathione concentrations to 50% of the maximal concentration. The data are shown in Table 8 and Table 9.

TABLE 8
Glutathione (GSH) EC50 and EC50 Relative to RTA 402.

| | | | GSH EC₅₀ | |
|---|---|---|---|---|
| | | | Relative to | |
| | | GSH EC₅₀ | RTA 402 | |
| | | (nM) | (fold)^{c} | # of |
| ID # | Structure | (Mean ± SD) | (Mean ± SD) | Repeats |
| CC2 | 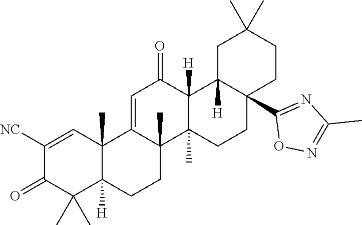 | 2.00 ± 1.24 | 0.80 ± 0.16 | 8 |
| T1 | 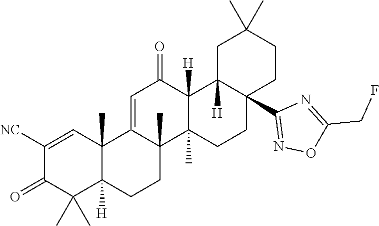 | 0.57 ± 0.22 | 0.35 ± 0.12 | 4 |
| T41 | 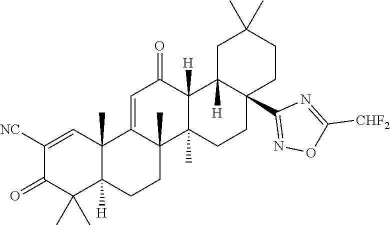 | 4.85 ± 0.36 | 2.38 ± 0.29 | 2 |
| T40 | 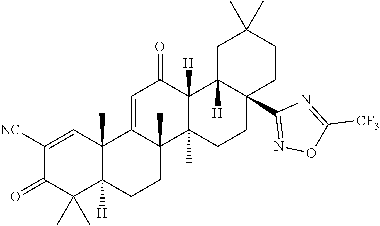 | 18.33 ± 2.19 | 8.96 ± 0.68 | 2 |
| T24 | 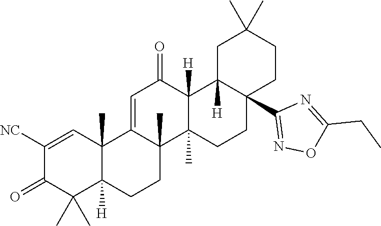 | 6.11 ± 2.23 | 2.53 ± 0.52 | 4 |
| T2 | 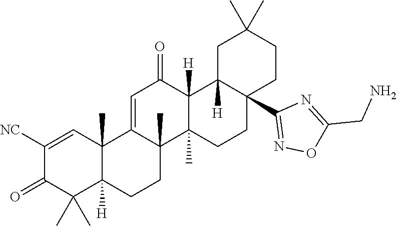 | 1.21 ± 1.15 | 0.47 ± 0.38 | 4 |
| T12 | 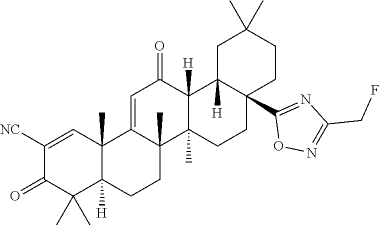 | 0.82 ± 0.40 | 0.50 ± 0.19 | 4 |
| T43 | 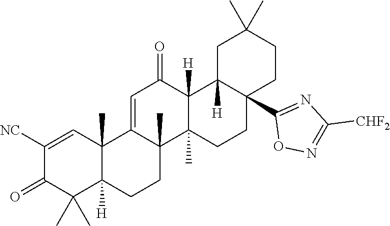 | 3.29 ± 0.55 | 1.60 ± 0.05 | 2 |
| T22 | 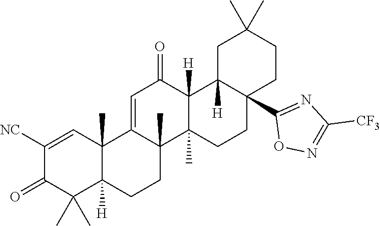 | 17.59 ± 0.59 | 8.72 ± 1.98 | 2 |
| T23 | 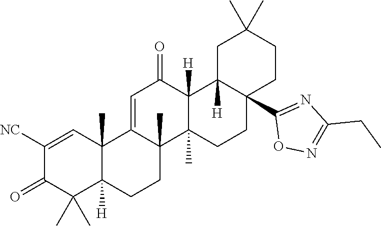 | 3.26 ± 0.51 | 2.05 ± 0.44 | 4 |
| CC1 | 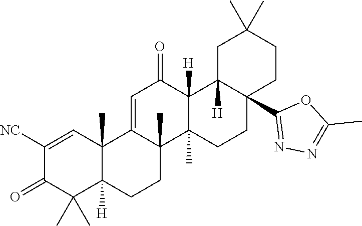 | 1.09 ± 0.13 | 0.70 ± 0.12 | 4 |
| T11 | 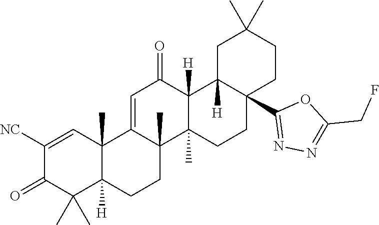 | 0.54 ± 0.29 | 0.32 ± 0.13 | 4 |
| T44 | 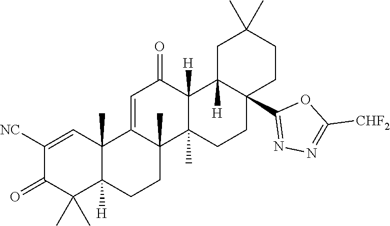 | 2.05 ± 0.64 | 1.04 ± 0.06 | 2 |
| T45 | 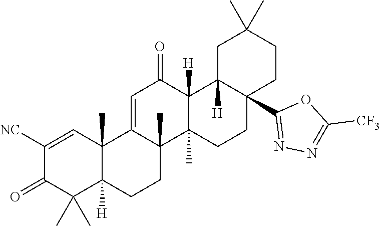 | 8.00 ± 1.37 | 4.14 ± 0.35 | 2 |
| T46 | 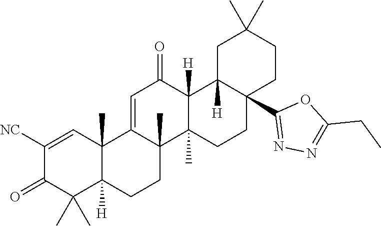 | 2.39 ± 0.29 | 1.24 ± 0.17 | 2 |
| CC3 | 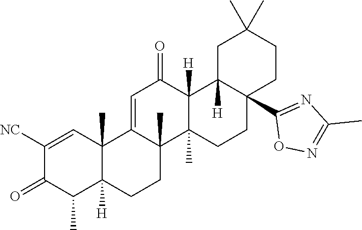 | 1.16 ± 0.53 | 0.43 ± 0.14 | 6 |
| T34 | 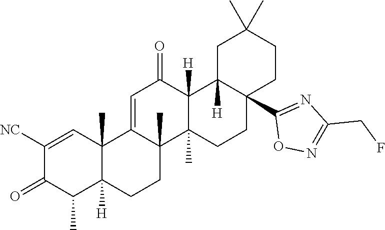 | 0.35 ± 0.06 | 0.18 ± 0.02 | 2 |
| T35 | 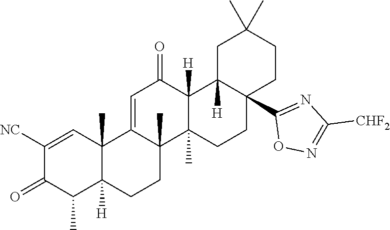 | 1.99 ± 0.42 | 1.02 ± 0.04 | 2 |
| T36 | 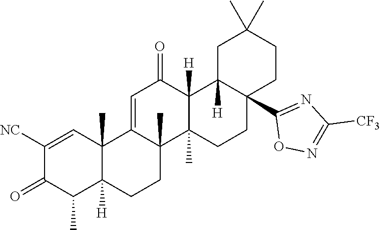 | 10.43 ± 1.62 | 5.40 ± 0.54 | 2 |
| T37 | 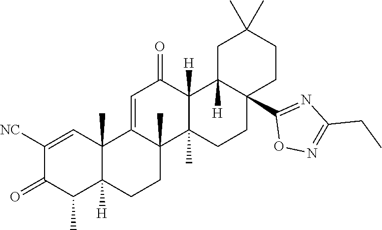 | 1.69 ± 0.19 | 0.88 ± 0.13 | 2 |
| T3 | 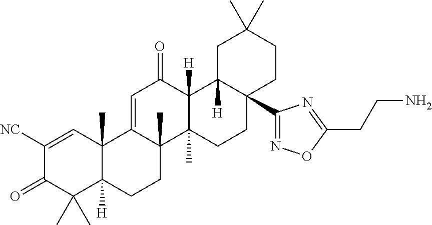 | 1.45 ± 0.28 | 0.54 ± 0.06 | 2 |
| T4 | 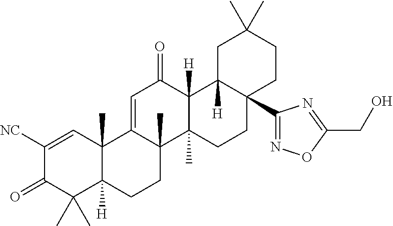 | 2.46 ± 1.80 | 0.89 ± 0.61 | 2 |
| T5 | 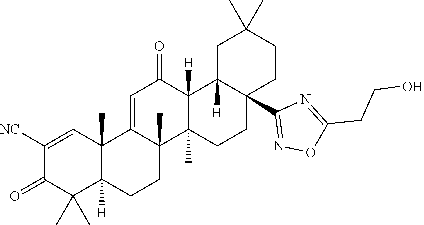 | 1.77 ± 0.98 | 0.65 ± 0.32 | 2 |
| T6 | 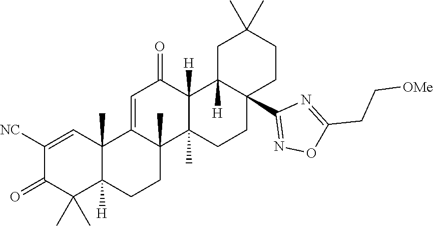 | 2.90 ± 0.96 | 1.07 ± 0.28 | 2 |
| T7 | 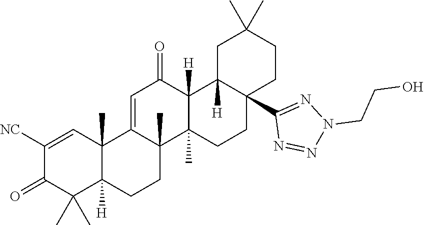 | 2.87 ± 3.17 | 0.80 ± 0.59 | 4 |
| T10 | 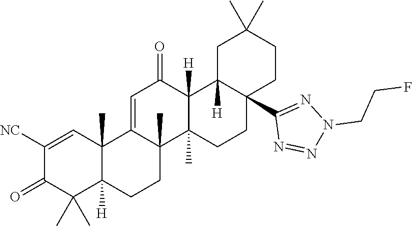 | 1.45 ± 0.17 | 1.14 ± 0.03 | 2 |
| T13 | 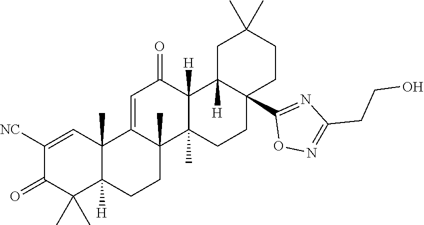 | 2.04 ± 1.94 | 0.55 ± 0.33 | 4 |
| T16 | 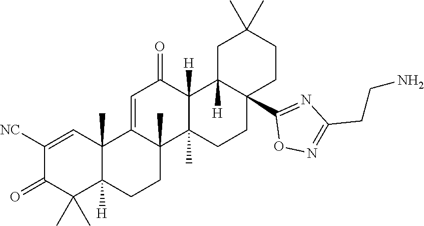 | 0.58 ± 0.04 | 0.46 ± 0.09 | 2 |
| T20 | 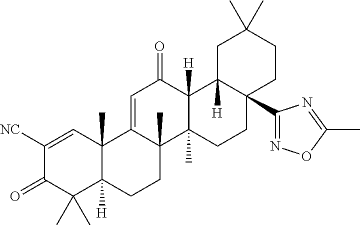 | 1.66 ± 0.66 | 0.96 ± 0.15 | 4 |
| T21 | 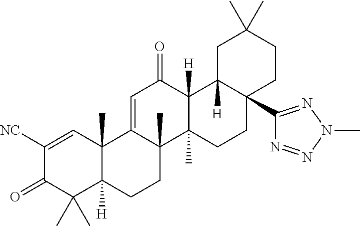 | 3.18 ± 2.22 | 0.95 ± 0.34 | 4 |
| T25 | 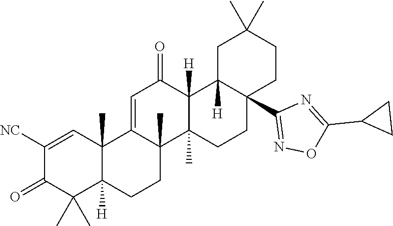 | 9.07 ± 1.55 | 3.36 ± 0.34 | 2 |

^{c}Average of ratios from replicate experiments

TABLE 9
Glutathione (GSH) EC50 Relative to Comparison Compounds.

| | | | GSH EC₅₀ | |
|---|---|---|---|---|
| | | | Relative to | |
| | | Comparison | CC in Same | |
| | | Compound | Experiment^{d} | # of |
| ID # | Structure | (CC) | (Mean ± SD) | Repeats |
| CC2 | 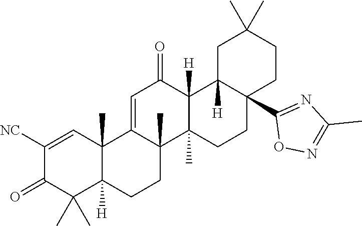 | CC2 | 1.00 | N/A |
| T1 | 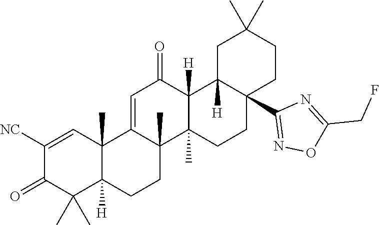 | CC2 | 0.50 ± 0.09 | 4 |
| T2 | 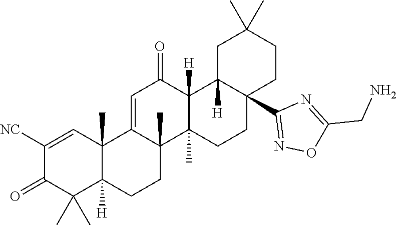 | CC2 | 0.41 ± 0.09 | 2 |
| T12 | 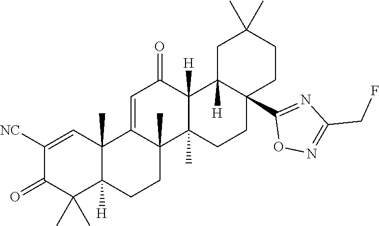 | CC2 | 0.69 ± 0.20 | 4 |
| CC1 | 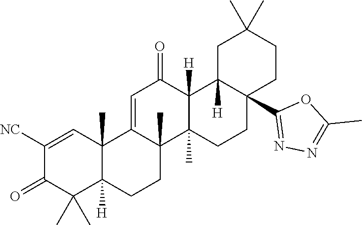 | CC1 | 1.00 | N/A |
| T11 | 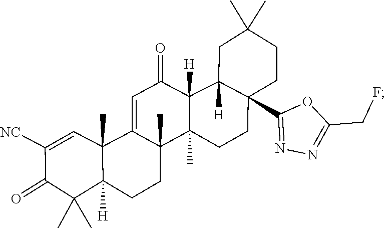 | CC1 | 0.42 ± 0.02 | 2 |
| T44 | 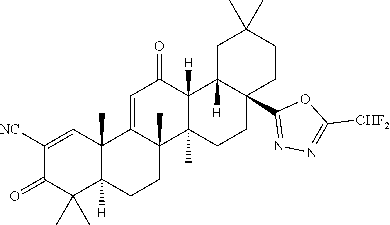 | CC1 | 1.72 ± 0.32 | 2 |
| CC3 | 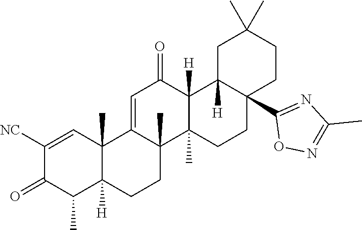 | CC3 | 1.00 | N/A |
| T34 | 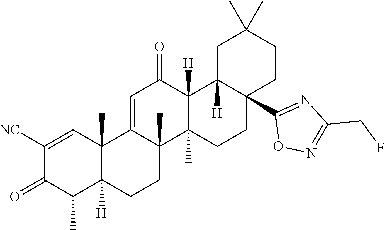 | CC3 | 0.58 ± 0.02 | 2 |
| T13 | 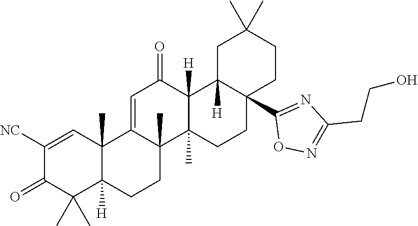 | CC2 | 0.62 ± 0.36 | 4 |
| T20 | 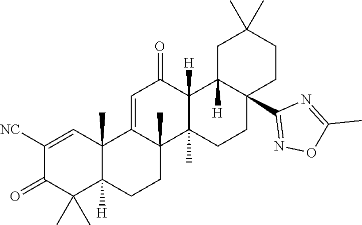 | CC2 | 1.41 ± 0.33 | 4 |

^{d}Average of ratios from experiments with direct comparisons.

### Example 5: Effect on Luciferase Reporter Activation

AREc32 reporter cell line (derived from human breast carcinoma MCF7 cells) was obtained was from CXR Bioscience Limited (Dundee, UK) and cultured in DMEM (low glucose) supplemented with 10% FBS, 1% penicillin/streptomycin, and 0.8 mg/ml Geneticin (G418). This cell line is stably transfected with a luciferase reporter gene under the transcriptional control of eight copies of the rat GSTA2 ARE sequence.

The effect of several compounds disclosed herein on luciferase reporter activation was assessed in the AREc32 reporter cell line (see Table 10 and Table 11). This cell line is derived from human breast carcinoma MCF-7 cells and is stably transfected with a luciferase reporter gene under the transcriptional control of eight copies of the antioxidant response element from the rat Gsta2 gene, an Nrf2 target gene (Frilling et al., 1990). AREc32 cells were plated in black 96-well plates in 200 L media at 20,000 cells per well. Twenty-four hours after plating, cells were treated with vehicle (DMSO) or test compounds at concentrations ranging from 0.03 to 1000 nM for nineteen hours. Media was removed and 100 μL of 1:1 mixture of the One-Glo Luciferase assay reagent and culture medium was added to each well. After incubation for 5 min at room temperature, the luminescence signal was measured on a PHERAstar plate reader. The EC_{2X} value was determined using Excel and GraphPad Prism software. The fold increase in luminescence signal for cells treated with each concentration of compound relative to cells treated with vehicle was determined and a dose-response curve was generated. The dose-response curve was fit using nonlinear regression analysis and used to extrapolate the EC_{2X} value. The EC_{2X} value is defined as the concentration of test compound required to increase the luminescence signal 2-fold above levels in vehicle-treated samples.

TABLE 10
AREc32 EC2X and EC2X Relative to RTA 402.

| | | | AREc32 EC_{2X} | |
|---|---|---|---|---|
| | | AREc32 | Relative to | |
| | | EC_{2X} | RTA 402 | |
| | | (nM) | (fold)^{e} | # of |
| ID # | Structure | (Mean ± SD) | (Mean ± SD) | Repeats |
| CC2 | 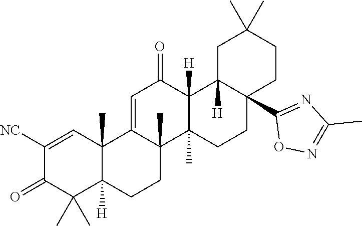 | 8.59 ± 1.88 | 0.74 ± 0.16 | 11 |
| T1 | 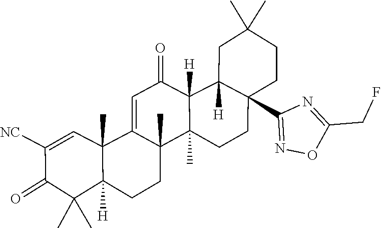 | 6.20 ± 1.37 | 0.46 ± 0.13 | 7 |
| T41 | 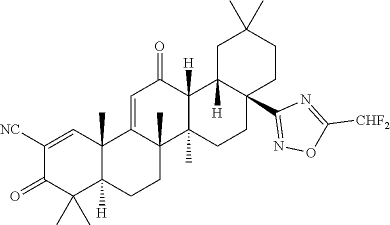 | 24.18 ± 3.36 | 1.52 ± 0.19 | 3 |
| T40 | 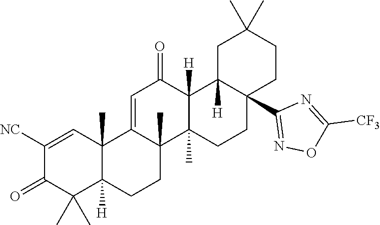 | 64.93 ± 12.64 | 4.76 ± 0.93 | 4 |
| T24 | 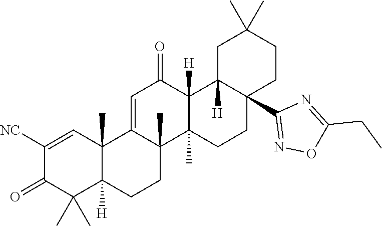 | 32.20 ± 2.59 | 2.50 ± 0.66 | 5 |
| T2 | 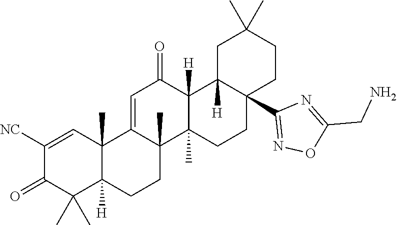 | 3.67 ± 0.28 | 0.28 ± 0.04 | 7 |
| T12 | 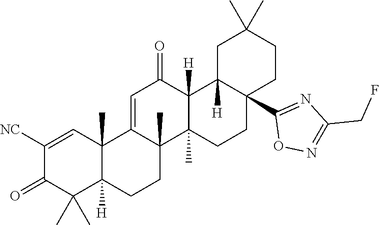 | 6.87 ± 1.57 | 0.53 ± 0.12 | 8 |
| T43 | 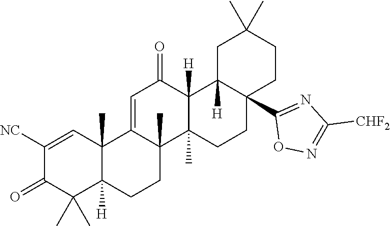 | 9.92 ± 2.32 | 0.62 ± 0.09 | 3 |
| T22 | 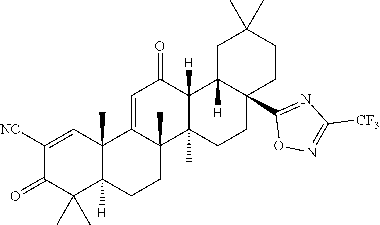 | 54.63 ± 8.83 | 3.45 ± 0.69 | 3 |
| T23 | 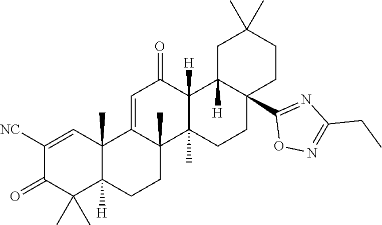 | 20.25 ± 2.51 | 1.43 ± 0.26 | 5 |
| CC1 | 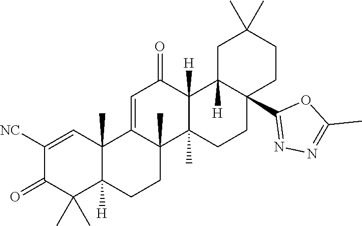 | 10.63 ± 2.73 | 0.79 ± 0.24 | 5 |
| T11 | 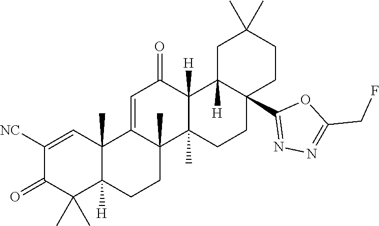 | 3.23 ± 0.77 | 0.25 ± 0.08 | 8 |
| T44 | 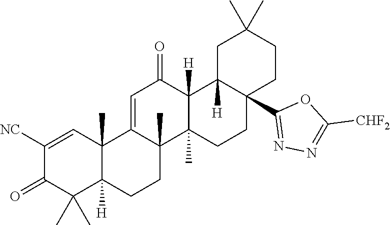 | 8.62 ± 0.45 | 0.57 ± 0.12 | 3 |
| T45 | 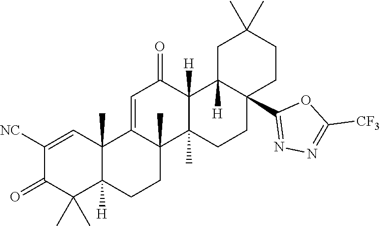 | 29.09 ± 2.09 | 1.95 ± 0.57 | 3 |
| T46 | 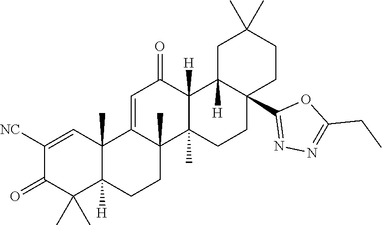 | 24.02 ± 1.50 | 1.60 ± 0.44 | 3 |
| CC3 | 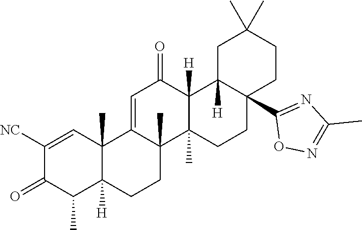 | 7.11 ± 1.15 | 0.66 ± 0.21 | 9 |
| T34 | 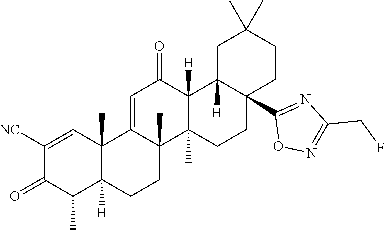 | 4.50 ± 0.66 | 0.36 ± 0.05 | 8 |
| T35 | 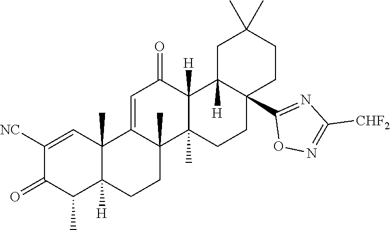 | 10.74 ± 1.02 | 0.73 ± 0.14 | 3 |
| T36 | 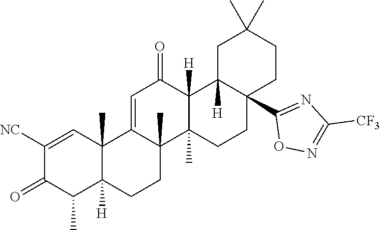 | 33.72 ± 6.99 | 2.25 ± 0.31 | 3 |
| T37 | 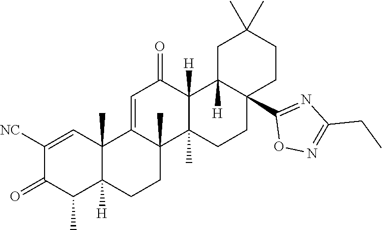 | 10.33 ± 1.35 | 0.69 ± 0.06 | 3 |
| CC4 | 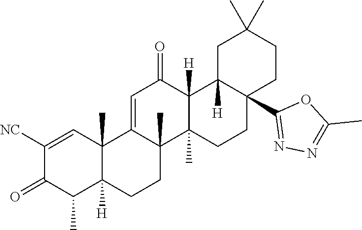 | 8.12 ± 3.53 | N/A | 3^{f} |
| T38 | 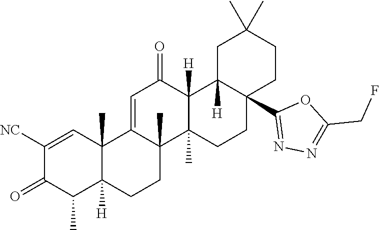 | 3.95 ± 1.12 | 0.43 ± 0.14 | 2 |
| T3 | 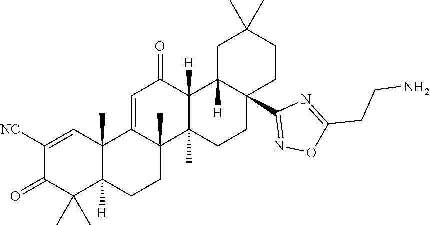 | 9.21 ± 3.66 | 0.91 ± 0.29 | 2 |
| T4 | 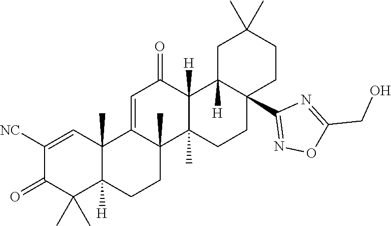 | 10.48 ± 1.03 | 1.04 ± 0.02 | 2 |
| T5 | 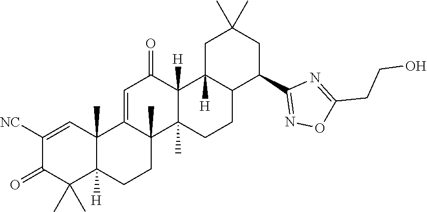 | 11.68 ± 2.80 | 1.16 ± 0.19 | 2 |
| T6 | 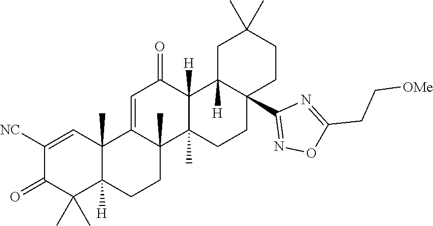 | 17.66 ± 0.10 | 1.77 ± 0.15 | 2 |
| T7 | 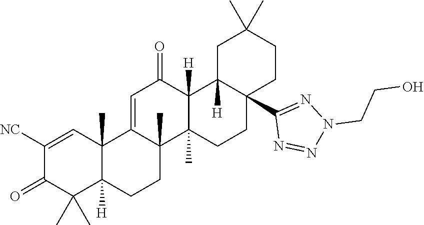 | 10.46 ± 3.66 | 1.10 ± 0.33 | 5 |
| T10 | 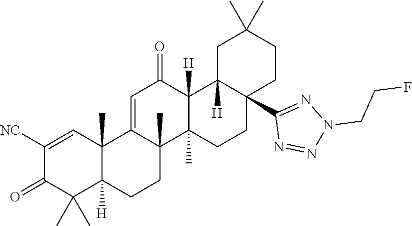 | 28.41 ± 9.42 | 2.41 ± 0.98 | 2 |
| T13 | 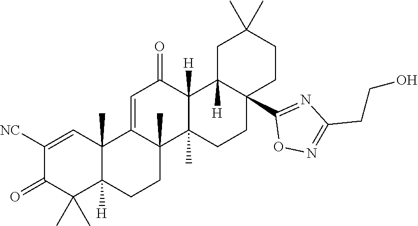 | 3.53 ± 0.98 | 0.37 ± 0.04 | 5 |
| T16 | 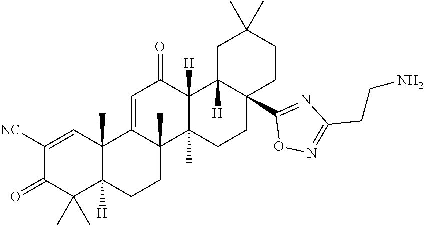 | 3.56 ± 0.31 | 0.30 ± 0.00 | 2 |
| T20 | 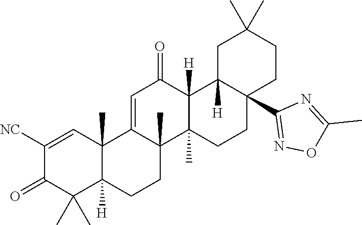 | 21.50 ± 2.85 | 1.50 ± 0.18 | 5 |
| T21 | 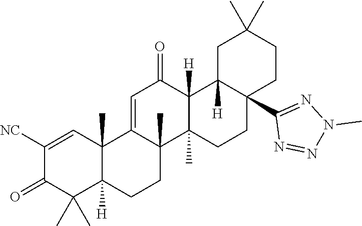 | 16.17 ± 4.20 | 1.67 ± 0.15 | 5 |
| T25 | 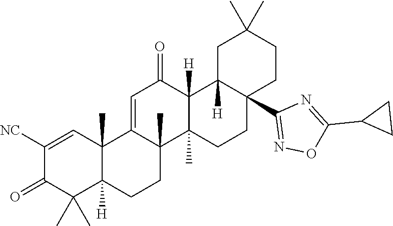 | 43.96 ± 1.20 | 4.39 ± 0.23 | 2 |
| T29 | 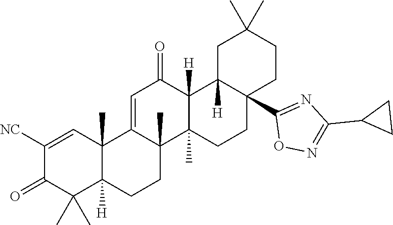 | 43.70 | 2.84 | 1 |
| T30 | 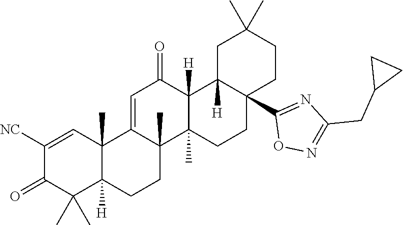 | 64.54 | 4.19 | 1 |
| T39 | 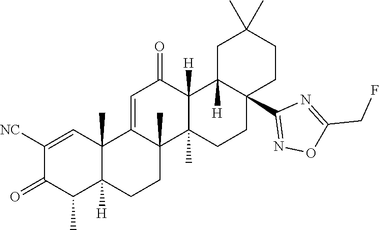 | 1.88 | 0.20 | 1 |
| T42 | 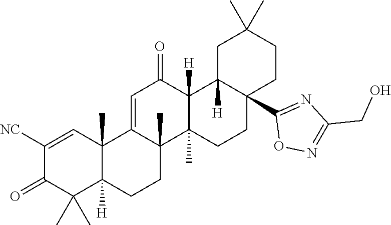 | 2.58 | 0.26 | 1 |

^{e}Average of ratios from replicate experiments.
^{f}# of repeats in this case refers only to AREc32 EC₂ₓ measurement. This compound was not run side by side by side against RTA 402 in the same experiment.

TABLE 11
AREc32 EX_{2X} Relative to Comparison Compounds.

| | | | AREc32 Relative | |
|---|---|---|---|---|
| | | Comparison | to CC in Same | |
| | | Compound | Experiment^{g} | # of |
| ID # | Structure | (CC) | (Mean ± SD) | Repeats |
| CC2 | 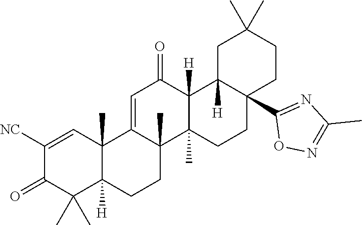 | CC2 | 1.00 | N/A |
| T1 | 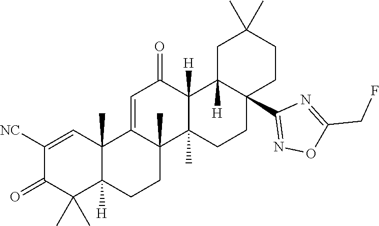 | CC2 | 0.62 ± 0.07 | 5 |
| T2 | 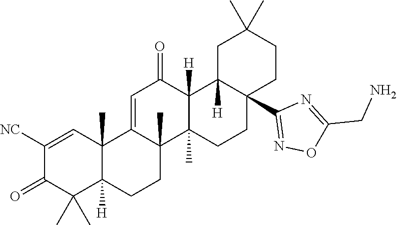 | CC2 | 0.43 ± 0.00 | 3 |
| T12 | 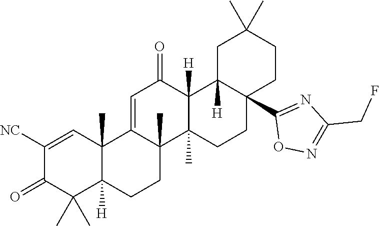 | CC2 | 0.75 ± 0.05 | 5 |
| T43 | 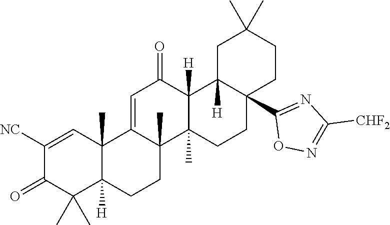 | CC2 | 1.09 ± 0.09 | 3 |
| T23 | 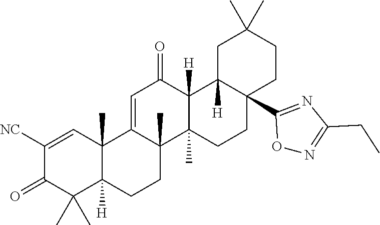 | CC2 | 2.41 ± 0.42 | 3 |
| CC1 | 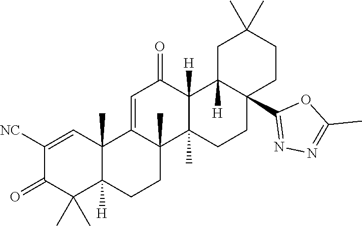 | CC1 | 1.00 | N/A |
| T11 | 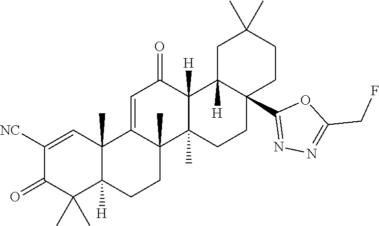 | CC1 | 0.27 ± 0.03 | 3 |
| T44 | 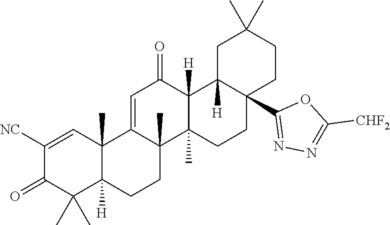 | CC1 | 0.91 ± 0.21 | 3 |
| CC3 | 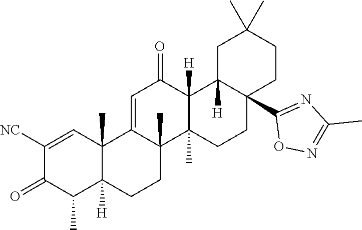 | CC3 | 1.00 | N/A |
| T34 | 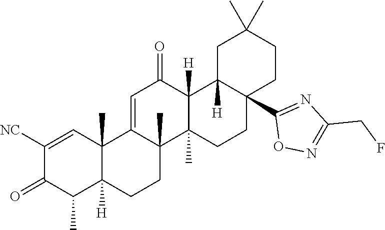 | CC3 | 0.76 ± 0.14 | 3 |
| T35 | 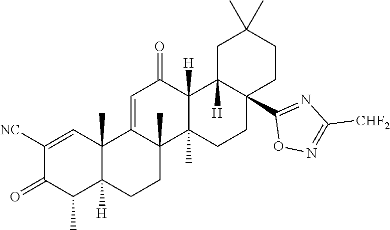 | CC3 | 1.66 ± 0.31 | 3 |
| T37 | 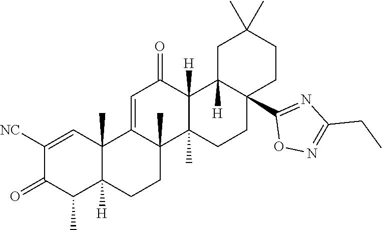 | CC3 | 1.58 ± 0.16 | 3 |
| T13 | 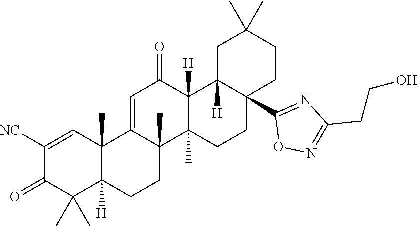 | CC2 | 0.44 ± 0.07 | 5 |

^{g}Average of ratios from experiments with direct comparisons.

All the compounds, formulations, and methods disclosed and claimed herein can be made and executed without undue experimentation in light of the present disclosure. While the compounds, formulations, and methods of the present disclosure have been described in terms of preferred embodiments, it will be apparent to those of skill in the art that variations may be applied to the compounds, formulations, and methods, as well as in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
U.S. Pat. No. 6,326,507
U.S. Pat. No. 6,974,801
U.S. Pat. No. 7,435,755
U.S. Pat. No. 7,795,305
U.S. Pat. No. 7,863,327
U.S. Pat. No. 7,915,402
U.S. Pat. No. 7,943,778
U.S. Pat. No. 8,034,955
U.S. Pat. No. 8,071,632
U.S. Pat. No. 8,124,656
U.S. Pat. No. 8,124,799
U.S. Pat. No. 8,129,429
U.S. Pat. No. 8,338,618
U.S. Pat. No. 8,394,967
U.S. Pat. No. 8,440,820
U.S. Pat. No. 8,440,854
U.S. Pat. No. 8,455,544
U.S. Pat. No. 8,586,775
U.S. Pat. No. 8,993,640
U.S. Pat. No. 9,090,574
U.S. Pat. No. 9,102,681
U.S. Pat. No. 9,249,089
U.S. Pat. No. 9,278,912
U.S. Pat. No. 9,278,913
U.S. Pat. No. 9,290,536
U.S. Pat. No. 9,512,094
U.S. Pat. No. 9,556,222
U.S. Pat. No. 9,593,074
U.S. Pat. No. 9,670,147
U.S. Pat. No. 9,701,709
U.S. Pat. No. 9,757,359
U.S. Pat. No. 9,856,286
U.S. Pat. No. 9,889,143
U.S. Pat. No. 10,093,614
U.S. Pat. No. 10,105,372
U.S. Pat. No. 10,398,711
U.S. Pat. No. 10,501,489
U.S. Pat. No. 10,556,858
WO 2012/125488
WO 2014/040056
WO 2017/053868
WO 2018/089539
Abraham and Kappas, Free Radical Biol. Med., 39:1-25, 2005.
Ahmad et al., Cancer Res., 68:2920-2926, 2008.
Ahmad et al., J. Biol. Chem., 281:35764-35769, 2006.
Anderson, Practical Process Research & Development—A Guide for Organic Chemists, 2^{nd} ed., Academic Press, New York, 2012.
Araujo et al., J. Immunol., 171(3):1572-1580, 2003.
Bach, Hum. Immunol., 67(6):430-432, 2006.
Bachhawat and Yadav, IUBMB Life, 70(7):585-592, 2018.
Chauhan and Chauhan, Pathophysiology, 13(3):171-181 2006.
Dickerson et al., Prog Neuropsychopharmacol Biol. Psychiatry, Mar. 6, 2007.
Dinkova-Kostova et al., Proc. Natl. Acad. Sci. USA, 102(12):4584-4589, 2005.
Dudhgaonkar et al., Eur. J. Pain, 10(7):573-579, 2006.
Favaloro, et al., J. Med. Chem., 45:4801-4805, 2002.
Florczyk et al., Pharmacol. Rep., 60(1):38-48, 2008.
Forstermann, Biol. Chem., 387:1521, 2006.
Frilling et al., Proc Natl Acad Sci USA, 87(16):6258-6262, 1990.
Handbook of Pharmaceutical Salts: Properties, and Use, Stahl and Wermuth Eds., Verlag Helvetica Chimica Acta, 2002.
Hanson et al., BMC Medical Genetics, 6(7), 2005.
Honda et al. Bioorg. Med. Chem. Lett., 12:1027-1030, 2002.
Honda et al., Bioorg. Med. Chem. Lett., 16(24):6306-6309, 2006.
Honda et al., Bioorg. Med. Chem. Lett., 7:1623-1628, 1997.
Honda et al., Bioorg. Med. Chem. Lett., 8(19):2711-2714, 1998.
Honda et al., Bioorg. Med. Chem. Lett., 9(24):3429-3434, 1999.
Honda et al., J. Med. Chem., 43:4233-4246, 2000a.
Honda et al., Org. Biomol. Chem., 1:4384-4391, 2003.
Honda et al., J. Med. Chem., 43:1866-1877, 2000b.
Honda et al., Bioorg. Med. Chem. Lett., 12(7):1027-1030, 2002.
Honda et al., J. Med. Chem., 54(6):1762-1778, 2011.
Hong et al., Clin. Cancer Res., 18(12):3396-3406, 2012.
Ishikawa et al., Circulation, 104(15):1831-1836, 2001.
Kawakami et al., Brain Dev., 28(4):243-246, 2006.
Kendall-Tackett, Trauma Violence Abuse, 8(2):117-126, 2007.
Kruger et al., J. Pharmacol. Exp. Ther., 319(3):1144-1152, 2006.
Lee et al., Glia., 55(7):712-22, 2007.
Lencz et al., Mol. Psychiatry, 12(6):572-80, 2007.
Liby et al., Cancer Res., 65(11):4789-4798, 2005.
Liby et al., Mol. Cancer Ther., 6(7):2113-9, 2007b.
Liby et al., Nat. Rev. Cancer, 7(5):357-356, 2007a.
Liu et al., FASEB J., 20(2):207-216, 2006.
Lu et al., J. Clin. Invest., 121(10):4015-29, 2011.
McIver et al., Pain, 120(1-2):161-9, 2005.
Morris et al., J. Mol. Med., 80(2):96-104, 2002.
Morse and Choi, Am. J. Respir. Crit. Care Med., 172(6):660-670, 2005.
Morse and Choi, Am. J. Respir. Crit. Care Med., 27(1):8-16, 2002.
Motterlini and Foresti, Am. J. Physiol. Cell Physiol., 312:C302-C313, 2017.
Pall, Med. Hypoth., 69:821-825, 2007.
Pergola et al., N. Engl. J. Med., 365:327-336, 2011.
Place et al., Clin. Cancer Res., 9(7):2798-806, 2003.
Rajakariar et al., Proc. Natl. Acad. Sci. USA, 104(52):20979-84, 2007.
Reagan-Shaw et al., FASEB J., 22(3):659-661, 2008
Reisman et al., Arch. Dermatol. Res., 306(5):447-454, 2014.
Ross et al., Am. J. Clin. Pathol., 120(Suppl):553-71, 2003.
Ross et al., Expert Rev. Mol. Diagn., 3(5):573-585, 2003.
Ruster et al., Scand. J. Rheumatol., 34(6):460-3, 2005.
Sacerdoti et al., Curr Neurovasc Res. 2(2):103-111, 2005.
Salvemini et al., J. Clin. Invest., 93(5):1940-1947, 1994.
Sarchielli et al., Cephalalgia, 26(9):1071-1079, 2006.
Satoh et al., Proc. Natl. Acad. Sci. USA, 103(3):768-773, 2006.
Schulz et al., Antioxid. Redox. Sig., 10:115, 2008.
Smith, March's Advanced Organic Chemistry: Reactions, Mechanisms, and Structure, 7^{th} Ed., Wiley, 2013.
Sporn et al., J. Exp. Med., 184(4):1425-1433, 1996.
Suh et al., Cancer Res., 58:717-723, 1998.
Suh et al., Cancer Res., 59(2):336-341, 1999.
Szabo et al., Nature Rev. Drug Disc., 6:662-680, 2007.
Takahashi et al., Cancer Res., 57:1233-1237, 1997.
Tamir and Tannebaum, Chem. Res. Toxicol., 9(5):821-827, 1996.
Thimmulappa et al., Cancer Res., 62(18):5196-5203, 2002.
Tamir and Tannebaum, Biochim. Biophys. Acta, 1288:F31-F36, 1996.
Xie et al., J Biol Chem. 270(12):6894-6900, 1995.
Zhou et al., Am. J. Pathol., 166(1):27-37, 2005.

1. A compound of the formula: 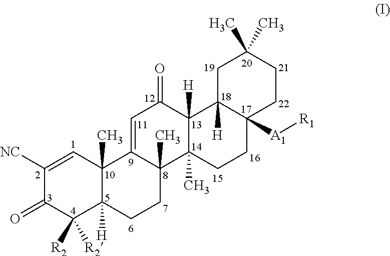
wherein:
A₁ is -heteroarenediyl_{(C≤3)}- selected from among 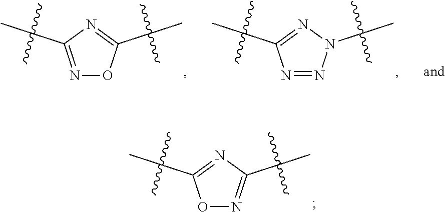 [SMILES: *c1noc(*)n1 ; *c1nnn(*)n1 ; *c1noc(*)n1]
R₁ is a monohydroxy-substituted alkyl_{(C≤3)}; and
R₂ and R₂′ are each methyl;
or a pharmaceutically acceptable salt thereof.

2.-4. (canceled)

5. The compound according to claim 1, wherein -A₁-R₁ is:

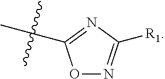

6. The compound according to claim 1, wherein -A₁-R₁ is:

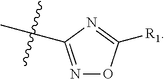

7. The compound according to claim 1, wherein -A₁-R₁ is:

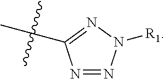

8.-10. (canceled)

11. The compound of claim 1, wherein R₁ is monopolar-substituted ethyl.

12. The compound of claim 1, wherein R₁ is monopolar-substituted methyl.

13.-23. (canceled)

24. The compound of claim 11, wherein R₁ is 2-hydroxyethyl.

25.-29. (canceled)

30. The compound according to claim 1, further defined as: 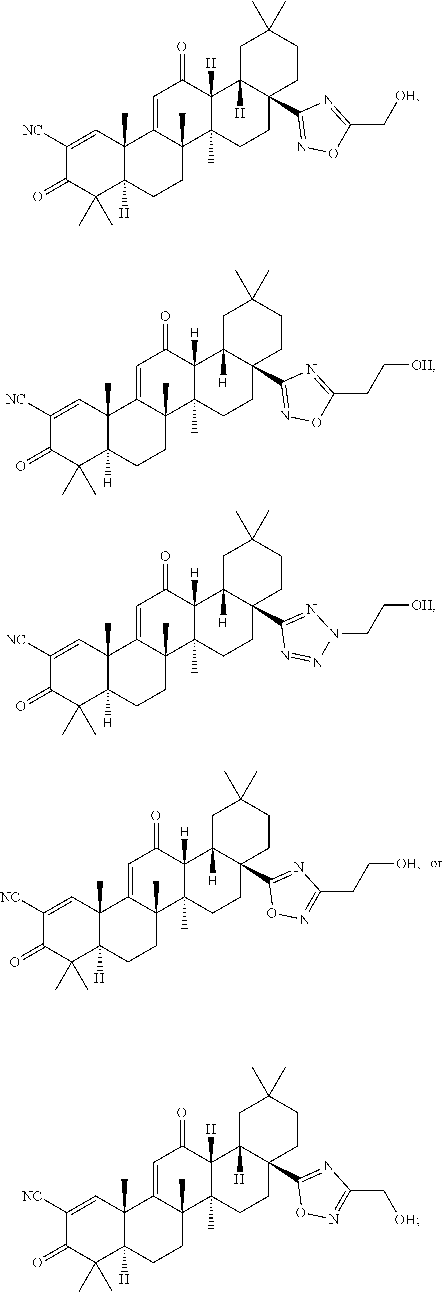
or a pharmaceutically acceptable salt of any of these formulas.

31.-33. (canceled)

34. The compound according to claim 30, further defined as: 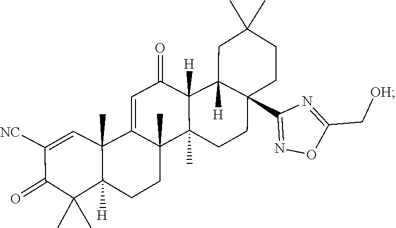
or a pharmaceutically acceptable salt thereof.

35. The compound according to claim 30, further defined as: 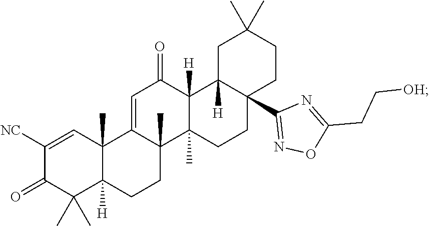
or a pharmaceutically acceptable salt thereof.

36. The compound according to claim 30, further defined as: 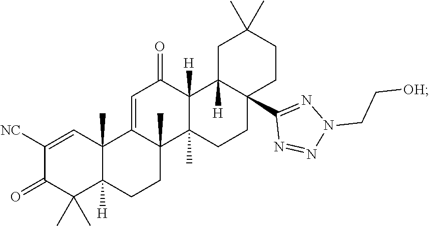
or a pharmaceutically acceptable salt thereof.

37. The compound according to claim 30, further defined as: 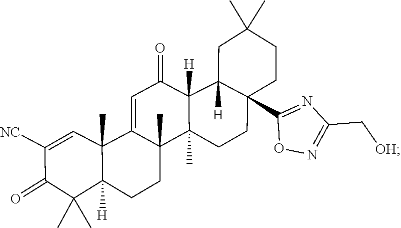
or a pharmaceutically acceptable salt thereof.

38. The compound according to claim 30, further defined as: 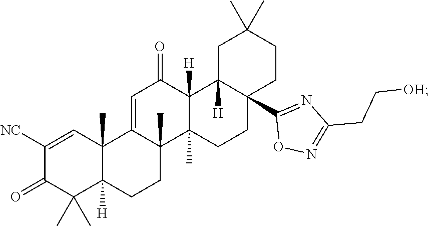
or a pharmaceutically acceptable salt thereof.

39.-49. (canceled)

50. A pharmaceutical composition comprising:
(A) a compound according to claim 1; and
(B) an excipient.

51. The pharmaceutical composition of claim 50, wherein the pharmaceutical composition is formulated for administration orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

52.-78. (canceled)
